(19) United States
(12) Patent Application Publication
(10) Pub. No.: US 2015/0322118 A1
(43) Pub. Date: Nov. 12, 2015

(54) RECOMBINANT CLOSTRIDIAL NEUROTOXINS WITH ENHANCED MEMBRANE LOCALIZATION

(71) Applicant: MERZ PHARMA GMBH & CO. KGAA, Frankfurt am Main (DE)

(72) Inventors: Gerhard GROER, Kufstein (AT); Jurgen FREVERT, Berlin (DE); Fred HOFMANN, Potsdam (DE); Michael SCHMIDT, Potsdam (DE)

(21) Appl. No.: 14/649,778

(22) PCT Filed: Dec. 5, 2013
(86) PCT No.: PCT/EP2013/003680

§ 371 Date: Jun. 4, 2015

Related U.S. Application Data
(60) Provisional application No. 61/733,525, filed on Dec. 5, 2012.

(30) Foreign Application Priority Data
Dec. 5, 2012 (EP) 12008148.4

(51) Int. Cl. C07K 14/33; C07K 14/47

(52) U.S. Cl. CPC C07K 14/33; C07K 14/4728; C07K 2319/31; C07K 2319/01; A61K 38/00

(57) ABSTRACT

This invention relates to novel recombinant clostridial neurotoxins exhibiting increased membrane localization and to methods for the manufacture of such recombinant clostridial neurotoxins. These methods comprise the steps of inserting a nucleic acid sequence coding for a C2 domain into a nucleic acid sequence coding for a parental clostridial neurotoxin and expression of the recombinant nucleic acid sequence comprising the C2 domain in a host cell. The invention further relates to novel recombinant single-chain precursor clostridial neurotoxins used in such methods, nucleic acid sequences encoding such recombinant single-chain precursor clostridial neurotoxins, and pharmaceutical compositions comprising the recombinant clostridial neurotoxin with increased membrane localization.

### FIELD OF THE INVENTION

This invention relates to novel recombinant clostridial neurotoxins exhibiting increased membrane localization and to methods for the manufacture of such recombinant clostridial neurotoxins. These methods comprise the steps of inserting a nucleic acid sequence coding for a C2 domain into a nucleic acid sequence coding for a parental clostridial neurotoxin and expression of the recombinant nucleic acid sequence comprising the C2 domain in a host cell. The invention further relates to novel recombinant single-chain precursor clostridial neurotoxins used in such methods, nucleic acid sequences encoding such recombinant single-chain precursor clostridial neurotoxins, and pharmaceutical compositions comprising the recombinant clostridial neurotoxin with increased membrane localization.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TxNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin acts mainly in the central nervous system, preventing the release of the inhibitory neurotransmitters GABA (gamma-aminobutyric acid) and glycine by degrading the protein synaptobrevin. The consequent overactivity in the muscles results in generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic types, termed BoNT/A through BoNT/G. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TxNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TxNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

Clostridial neurotoxins display variable durations of action that are serotype specific. The clinical therapeutic effect of BoNT/A lasts approximately 3 months for neuromuscular disorders and 6 to 12 months for hyperhidrosis. The effects of BoNT/E, on the other hand, last less than 4 weeks. The longer lasting therapeutic effect of BoNT/A makes it preferable for clinical use compared to the other serotypes, for example serotypes B, C₁, D, E, F and G. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with either 450 kDa or with 900 kDa are obtainable from cultures of Clostridium botulinum.

In recent years, botulinum neurotoxins have been used as therapeutic agents in the treatment of dystonias and spasms. Preparations comprising botulinum toxin complexes are commercially available, e.g. from Ipsen Ltd (Dysport) or Allergan Inc. (Botox®). A high purity neurotoxic component, free of any complexing proteins, is for example available from Merz Pharmaceuticals GmbH, Frankfurt (Xeomin).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

At present, clostridial neurotoxins are still predominantly produced by fermentation processes using appropriate Clostridium strains. However, industrial production of clostridial neurotoxin from anaerobic Clostridium culture is a cumbersome and time-consuming process. Due to the high toxicity of the final product, the procedure must be performed under strict containment. During the fermentation process, the single-chain precursors are proteolytically cleaved by an unknown clostridial protease to obtain the biologically active di-chain clostridial neurotoxin. The degree of neurotoxin activation by proteolytic cleavage varies between different strains and neurotoxin serotypes, which is a major consideration for the manufacture due to the requirement of neurotoxin preparations with a well-defined biological activity. Furthermore, during fermentation processes using Clostridium strains the clostridial neurotoxins are produced as protein complexes, in which the neurotoxic component is embedded by accessory proteins. These accessory proteins have no beneficial effect on biological activity or duration of effect. They can however trigger an immune reaction in the patient, resulting in immunity against the clostridial neurotoxin. Manufacture of recombinant clostridial neurotoxins, which are not embedded by auxiliary proteins, might therefore be advantageous.

Methods for the recombinant expression of clostridial neurotoxins in E. coli are well known in the art (see, for example, WO 00/12728, WO 01/14570, or WO 2006/076902). Furthermore, clostridial neurotoxins have been expressed in eukaryotic expression systems, such as in Pichia pastoris, Pichia methanolica, Saccharomyces cerevisiae, insect cells and mammalian cells (see WO 2006/017749).

Recombinant clostridial neurotoxins may be expressed as single-chain precursors, which subsequently have to be proteolytically cleaved to obtain the final biologically active clostridial neurotoxin. Thus, clostridial neurotoxins may be expressed in high yield in rapidly-growing bacteria as relatively non-toxic single-chain polypeptides.

Furthermore, it might be advantageous to modify clostridial neurotoxin characteristics regarding biological activity, cell specificity, antigenic potential and duration of effect by genetic engineering to obtain neurotoxins with new therapeutic properties in specific clinical areas. Genetic modification of clostridial neurotoxins might allow altering the mode of action or expanding the range of therapeutic targets.

WO 96/39166 discloses analogues of botulinum toxin comprising amino acid residues which are more resistant to degradation in neuromuscular tissue.

Patent family based on WO 02/08268 (including family member U.S. Pat. No. 6,903,187) discloses a clostridial neurotoxin comprising a structural modification selected from addition or deletion of a leucine-based motif, which alters the biological persistence of the neurotoxin (see also: Fernandez-Salas et al., Proc. Natl. Acad. Sci. U.S.A. 101 (2004) 3208-3213; Wang et al., J. Biol. Chem. 286 (2011) 6375-6385). Fernandez-Salas et al. initially hypothesized that the increased persistence was due to the membrane-binding properties of the dileucine motif (see Fernandez-Salas et al., loc. cit., p. 3211 and 3213). Wang et al. mention this membrane theory (see Wang et al., loc. cit., p. 6376, left column, last full paragraph, and p. 6383, first full paragraph of “Discussion”), but favor an alternative theory: the protection from degradation by proteolysis (see Wang et al., loc. cit., p. 6384, left column, lines 27ff).

US 2002/0127247 describes clostridial neurotoxins comprising modifications in secondary modification sites and exhibiting altered biological persistence.

Botulinum toxin variants exhibiting longer biological half lives in neuromuscular tissue than naturally occurring botulinum toxins would be advantageous in order to reduce administration frequency and the incidence of neutralising antibody generation since immunologic tolerance to botulinum toxin is correlated with cumulative doses.

Furthermore, BoNT serotypes exhibiting a short duration of action could potentially be effectively used in clinical applications, if their biological persistence could be enhanced. Modified BoNT/E with an increased duration of action could potentially be used in patients exhibiting an immune reaction against BoNT/A. Moreover, BoNT/E was shown to induce a more severe block of pain mediator release from sensory neurons than BoNT/A. In clinical applications where BoNT/A provides only partial pain relief or in just a subset of patients such as headache, or were BoNT/E has been found to be more effective than BoNT/A but gives only short-term therapy, such as epilepsy, BoNT/E with an increased duration of effect might prove useful.

There is a strong demand to produce clostridial neurotoxins with an increased duration of effect, in order to allow for reduction of administration frequency and exploitation of the therapeutic potential of BoNT serotypes which have so far been considered impractical for clinical application due to their short half-lives. Ideally, the duration of effect of a particular clostridial neurotoxin can be adjusted in a tailor-made fashion in order to address any particular features and demands of a given indication, such as amount of neurotoxin being administered, frequency of administration etc. To date, such aspects have not been solved satisfactorily.

### OBJECTS OF THE INVENTION

It was an object of the invention to provide recombinant clostridial neurotoxins exhibiting an increased duration of effect and to establish a reliable and accurate method for manufacturing and obtaining such recombinant clostridial neurotoxins. In particular, the generation of recombinant clostridial neurotoxins, which are protected from cytosolic degradation due to their enhanced association with cellular membranes, is intended by the invention. Such a method and novel precursor clostridial neurotoxins used in such methods would serve to satisfy the great need for recombinant clostridial neurotoxins exhibiting an increased duration of effect.

### SUMMARY OF THE INVENTION

The naturally occurring botulinum toxin serotypes display highly divergent durations of effect, probably due to their distinct subcellular localization. BoNT/A exhibiting the longest persistence was shown to localize in the vicinity of the plasma membrane of neuronal cells, whereas the short-duration BoNT/E serotype is cytosolic. Enhancing binding affinity of clostridial neurotoxins to the plasma membrane might thus prove efficient in protecting them and increasing their duration of effect.

So far, no modified clostridial neurotoxins exhibiting enhanced membrane localisation are available. Surprisingly, it has been found that recombinant clostridial neurotoxins with an increased tendency to associate with cellular membranes can be obtained by cloning a sequence encoding a C2 domain into a gene encoding a parental clostridial neurotoxin, and by subsequent heterologous expression of the generated construct in recombinant host cells.

Thus, in one aspect, the present invention relates to a recombinant clostridial neurotoxin comprising a C2 domain.

In another aspect, the present invention relates to a pharmaceutical composition comprising the recombinant clostridial neurotoxin of the present invention.

In yet another aspect, the present invention relates to the use of the composition of the present invention for cosmetic treatment.

In another aspect, the present invention relates to a method for the generation of the recombinant clostridial neurotoxin of the present invention, comprising the step of obtaining a recombinant nucleic acid sequence encoding a recombinant single-chain precursor clostridial neurotoxin by the insertion of a nucleic acid sequence encoding said C2 domain into a nucleic acid sequence encoding a parental clostridial neurotoxin.

In another aspect, the present invention relates to a recombinant single-chain precursor clostridial neurotoxin comprising a C2 domain.

In another aspect, the present invention relates to a nucleic acid sequence encoding the recombinant single-chain precursor clostridial neurotoxin of the present invention.

In another aspect, the present invention relates to a method for obtaining the nucleic acid sequence of the present invention, comprising the step of inserting a nucleic acid sequence encoding a C2 domain into a nucleic acid sequence encoding a parental clostridial neurotoxin.

In another aspect, the present invention relates to a vector comprising the nucleic acid sequence of the present invention, or the nucleic acid sequence obtainable by the method of the present invention.

In another aspect, the present invention relates to a recombinant host cell comprising the nucleic acid sequence of the present invention, the nucleic acid sequence obtainable by the method of the present invention, or the vector of the present invention.

In another aspect, the present invention relates to a method for producing the recombinant single-chain precursor clostridial neurotoxin of the present invention, comprising the step of expressing the nucleic acid sequence of the present invention, or the nucleic acid sequence obtainable by the method of the present invention, or the vector of the present invention in a recombinant host cell, or cultivating the recombinant host cell of the present invention under conditions that result in the expression of said nucleic acid sequence.

### DETAILED DESCRIPTION OF THE INVENTION

The present invention may be understood more readily by reference to the following detailed description of the invention and the examples included therein.

In one aspect, the present invention relates to a recombinant clostridial neurotoxin comprising a C2 domain.

In the context of the present invention, the term “clostridial neurotoxin” refers to a natural neurotoxin obtainable from bacteria of the class Clostridia, including Clostridium tetani and Clostridium botulinum, or to a neurotoxin obtainable from alternative sources, including from recombinant technologies or from genetic or chemical modification. Particularly, the clostridial neurotoxins have endopeptidase activity.

Clostridial neurotoxins are produced as single-chain precursors that are proteolytically cleaved by an unknown clostridial endoprotease within the loop region to obtain the biologically active disulfide-linked di-chain form of the neurotoxin, which comprises two chain elements, a functionally active light chain and a functionally active heavy chain, where one end of the light chain is linked to one end of the heavy chain not via a peptide bond, but via a disulfide bond.

In the context of the present invention, the term “clostridial neurotoxin light chain” refers to that part of a clostridial neurotoxin that comprises an endopeptidase activity responsible for cleaving one or more proteins that is/are part of the so-called SNARE-complex involved in the process resulting in the release of neurotransmitter into the synaptic cleft: In naturally occurring clostridial neurotoxins, the light chain has a molecular weight of approx. 50 kDa.

In the context of the present invention, the term “clostridial neurotoxin heavy chain” refers to that part of a clostridial neurotoxin that is responsible for entry of the neurotoxin into the neuronal cell: In naturally occurring clostridial neurotoxins, the heavy chain has a molecular weight of approx. 100 kDa.

In the context of the present invention, the term “functionally active clostridial neurotoxin chain” refers to a recombinant clostridial neurotoxin chain able to perform the biological functions of a naturally occurring Clostridium botulinum neurotoxin chain to at least about 50%, particularly to at least about 60%, to at least about 70%, to at least about 80%, and most particularly to at least about 90%, where the biological functions of clostridial neurotoxin chains include, but are not limited to, binding of the heavy chain to the neuronal cell, entry of the neurotoxin into a neuronal cell, release of the light chain from the di-chain neurotoxin, and endopeptidase activity of the light chain. Methods for determining a neurotoxic activity can be found, for example, in WO 95/32738, which describes the reconstitution of separately obtained light and heavy chains of tetanus toxin and botulinum toxin.

In the context of the present invention, the term “recombinant clostridial neurotoxin” refers to a composition comprising a clostridial neurotoxin that is obtained by expression of the neurotoxin in a heterologous cell such as E. coli, and including, but not limited to, the raw material obtained from a fermentation process (supernatant, composition after cell lysis), a fraction comprising a clostridial neurotoxin obtained from separating the ingredients of such a raw material in a purification process, an isolated and essentially pure protein, and a formulation for pharmaceutical and/or aesthetic use comprising a clostridial neurotoxin and additionally pharmaceutically acceptable solvents and/or excipients.

In the context of the present invention, the term “recombinant clostridial neurotoxin” further refers to a clostridial neurotoxin based on a parental clostridial neurotoxin comprising a heterologous C2 domain, i.e. a C2 domain that is not naturally occurring in a clostridial neurotoxin, in particular a C2 domain from a species other than Clostridium botulinum, in particular a C2 domain from a human protein.

In the context of the present invention, the term “C2 domain” refers to a widely occurring membrane targeting domain classified by InterPro (Hunter et al., InterPro in 2011: new developments in the family and domain prediction database. Nucleic Acids Res. 2012 January; 40(Database issue):D306-12) as “C2 calcium/lipid-binding domain, CaLB (IPR008973)”. Three subclasses of C2 domains are currently known: C2 calcium-dependent membrane targeting domain (IPR000008); phosphatidylinositol 3-kinase C2 (PI3K C2) domain (IPR002420); and tensin phosphatase, C2 domain (IPR014020). The C2 domains are domains of between about 100 and 160 amino acid residues found in many cellular peripheral proteins involved in signal transduction or membrane trafficking. C2 domains exhibit a wide range of lipid selectivity for the major components of cell membranes, including phosphatidylserine and phosphatidylcholine. They show similar tertiary structures consisting of an eight-stranded antiparallel β-sandwich. In many C2 domains, three Ca²⁺ binding loops are located at the end of the eight-stranded antiparallel β-sandwich, but in other C2 domains one or more of these loops may be missing. The tensin-type C2 domain, for example, lacks two of the three conserved loops that bind Ca²⁺. Due to local structural variation, particularly in the Ca²⁺ binding loops, C2 domains exhibit functional diversities and distinct subcellular localization patterns. A discussion of C2 domains, their binding to membrane components and ways of identifying C2 domains by homology searches can be found, for example, in Cho & Stahelin, (2006), Biochim Biophys Acta, 1761 (8), 838-849.

Comprehensive information about proteins is collected by the UniProt Consortium (The UniProt Consortium, Reorganizing the protein space at the Universal Protein Resource (UniProt), Nucleic Acids Research, 2012, Vol. 40, Database issue D71-D75), which maintains a database of proteins, which have been reviewed and annotated manually (UniProtKB/Swiss-Prot), and a database of proteins, which have not yet been reviewed and only been annotated automatically (UniProtKB/TrEMBL). Both databases are publically accessible via the internet, and are archived on a regular basis (Leinonen, R., Diez, F. G., Binns, D., Fleischmann, W., Lopez, R. and Apweiler, R. (2009) UniProt archive. Bioinformatics, 20, 3236-3237). As of November 2012, UniProtKB/Swiss-Prot and UniProtKB/TrEMBL list a total of 634 reviewed (C2 calcium-dependent membrane targeting domain: 573 entries; phosphatidylinositol 3-kinase C2 (PI3K C2) domain: 28 entries; tensin phosphatase, C2 domain: 33 entries), and 12,915 unreviewed proteins, respectively, which were identified to comprise at least one C2 domain. Table 5 contains a list of the 648 reviewed proteins. In total, there are 157 reviewed (UniProtKB/Swiss-Prot) (C2 calcium-dependent membrane targeting domain: 142 entries; phosphatidylinositol 3-kinase C2 (PI3K C2) domain: 5 entries; tensin phosphatase, C2 domain: 9 entries) and 317 unreviewed (UniProtKB/TrEMBL) human protein entries (organism: Homo sapiens). Table 6 contains a list of these 474 proteins (Table 6A: reviewed proteins; Table 6B: unreviewed proteins).

A C2 domain has been identified to be present in the alpha toxin of Clostridium perfringens (see: Chahinian et al., Curr. Protein Pept. Sci. 2000, 91-103; Guilluard et al., Molecular Microbiology 26 (1997) 867-876; and Naylor et al., J. Mol. Biol. 294 (1999) 757-770). The alpha toxin of Clostridium perfringens, however, is not a neurotoxin, but a phospholipase that generally acts on tissue cells by first binding via calcium-dependent interaction of a C-terminal C2 domain with the cell membrane.

In one embodiment, the membrane directing activity of a C2 domain is transferred to a parental clostridial neurotoxin by N- or C-terminally fusing said C2 domain to said parental clostridial neurotoxin light chain. N- or C-terminal fusion of a C2 domain to the parental clostridial neurotoxin light chain causes direct membrane binding of the clostridial neurotoxin light chain, thus affecting the subcellular localization of the catalytically active clostridial neurotoxin present in the recombinant clostridial neurotoxin.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In particular embodiments, said C2 domain is a C2 domain present in a protein listed in Table 5. In particular other embodiments, said C2 domain is a human C2 domain, particularly a human C2 domain present in a human protein listed in Table 6, particularly in Table 6A, particularly a human C2 domain present in a human protein selected from the list of: ABR; BAIP3; BCR; C2CD3; C2D1A; C2D1B; CAN5; CANE; CAPS1; CAPS2; CPNE1; CPNE2; CPNE3; CPNE4; CPNE5; CPNE6; CPNE7; CPNE8; CPNE9; CUO25; DAB2P; DOC2A; DOC2B; DYSF; ESYT1; ESYT2; ESYT3; FR1L5; FTM; HECW1; HECW2; ITCH; ITSN1; ITSN2; KPCA; KPCB; KPCE; KPCG; KPCL; MCTP1; MCTP2; MYOF; NEDD4; NED4L; NGAP; OTOF; P3C2A; P3C2B; P3C2G; PA24A; PA24B; PA24D; PA24E; PA24F; POLO; PERF; PLCB1; PLCB2; PLCB3; PLCB4; PLCD1; PLCD3; PLCD4; PLCE1; PLCG1; PLCG2; PLCH1; PLCH2; PLCL1; PLCL2; PLCZ1; RASA1; RASA2; RASA3; RASL1; RASL2; RFIP1; RFIP2; RFIP5; RGS3; RIMS1; RIMS2; RIMS3; RIMS4; RP3A; RPGR1; SMUF1; SMUF2; SY14L; SYGP1; SYT1; SYT10; SYT11; SYT12; SYT13; SYT14; SYT15; SYT16; SYT17; SYT2; SYT3; SYT4; SYT5; SYT6; SYT7; SYT8; SYT9; SYTL1; SYTL2; SYTL3; SYTL4; SYTL5; TAC2N; TOLIP; UN13A; UN13B; UN13C; UN13D; WWC2; WWP1; and WWP2. In particular embodiments, the C2 domain a human C2 domain present in a human protein selected from the list of: DOC2A; DOC2B; DYSF; ESYT1; ESYT2; ESYT3; FR1L5; KPCA; KPCB; KPCG; MYOF; NED4L; PLCD1; PLCD3; PLCZ1; RFIP1; RFIP2; RFIP5; RP3A; SYT1; SYT10; SYT2; SYT3; SYT4; SYT5; SYT6; SYT7; SYT9; and SYTL1.

In particular embodiments, said C2 domain has the amino acid sequence of one of the C2 domains listed in Table 1 (SEQ ID NOs: 1 to 32). More particularly said C2 domain is selected from SEQ-ID NOs. 1 to 2 and SEQ-ID NOs 27-29.

In particular embodiments C2 domain is a functional variant of a C2 domain present in a human protein, and/or listed in Table 1.

In the context of the present invention, the term “functional variant of a C2 domain” refers to a domain that differs in the amino acid sequence and/or the nucleic acid sequence encoding the amino acid sequence from a naturally occurring C2 domain, but is still functionally active. In this context “functionally active” or biologically active” means that said variant maintains the membrane directing activity of a C2 domain. In the context of the present invention, the term “functionally active” refers to the property of a recombinant C2 domain to perform the biological function of a naturally occurring C2 domain to at least about 50%, particularly to at least about 60%, to at least about 70%, to at least about 80%, and most particularly to at least about 90%, where the biological functions include, but are not limited to, binding of the C2 domain to the natural binding targets of C2 domains.

On the protein level, a functional variant will maintain key features of the corresponding C2 domain, such as key residues for maintaining the eight-stranded antiparallel β-sandwich structure, and/or key residues for maintaining the Ca²⁺ binding sites, but may contain one or more mutations comprising a deletion of one or more amino acids of the corresponding C2 domain, an addition of one or more amino acids of the corresponding C2 domain, and/or a substitution of one or more amino acids of the corresponding C2 domain. Particularly, said deleted, added and/or substituted amino acids are consecutive amino acids. According to the teaching of the present invention, any number of amino acids may be added, deleted, and/or substituted, as long as the functional variant remains biologically active. For example, 1, 2, 3, 4, 5, up to 10, up to 15, up to 25, up to 50, up to 100, up to 200, up to 400, up to 500 amino acids or even more amino acids may be added, deleted, and/or substituted. Accordingly, a functional variant of the C2 domain may be a biologically active fragment of a naturally occurring C2 domain. This C2 domain fragment may contain an N-terminal, C-terminal, and/or one or more internal deletion(s).

In particular embodiments, said C2 domain is inserted at (i) the N-terminus of the light chain of said parental clostridial neurotoxin or (ii) at the C-terminus of the light chain of said parental clostridial neurotoxin, and is thus present at (i) the N-terminus of the light chain of said recombinant clostridial neurotoxin or (ii) at the C-terminus of the light chain of said recombinant clostridial neurotoxin, respectively.

In particular embodiments, said clostridial neurotoxin is selected from (i) a Clostridium botulinum neurotoxin serotype A, B, C, D, E, F, and G, particularly Clostridium botulinum neurotoxin serotype A, C and E, or (ii) from a functional variant of a Clostridium botulinum neurotoxin of (i), or (iii) from a chimeric Clostridium botulinum neurotoxin, wherein the clostridial neurotoxin light chain and heavy chain are from different parental clostridial neurotoxin serotypes.

In the context of the present invention, the term “Clostridium botulinum neurotoxin serotype A, B, C, D, E, F, and G” refers to neurotoxins found in and obtainable from Clostridium botulinum. Currently, seven serologically distinct types, designated serotypes A, B, C, D, E, F, and G are known, including certain subtypes (e.g. A1, A2, A3, A4 and A5).

In particular embodiments the clostridial neurotoxin is selected from a Clostridium botulinum neurotoxin serotype A, C and E, or from a functional variant of any such Clostridium botulinum neurotoxin.

In the context of the present invention, the term “functional variant of a clostridial neurotoxin” refers to a neurotoxin that differs in the amino acid sequence and/or the nucleic acid sequence encoding the amino acid sequence from a clostridial neurotoxin, but is still functionally active. In the context of the present invention, the term “functionally active” refers to the property of a recombinant clostridial neurotoxin to exhibit a biological activity of at least about 50%, particularly to at least about 60%, at least about 70%, at least about 80%, and most particularly at least about 90% of the biological activity of a naturally occurring parental clostridial neurotoxin, i.e. a parental clostridial neurotoxin without C2 domain, where the biological functions include, but are not limited to, binding to the neurotoxin receptor, entry of the neurotoxin into a neuronal cell, release of the light chain from the two-chain neurotoxin, and endopeptidase activity of the light chain, and thus inhibition of neurotransmitter release from the affected nerve cell.

On the protein level, a functional variant will maintain key features of the corresponding clostridial neurotoxin, such as key residues for the endopeptidase activity in the light chain, or key residues for the attachment to the neurotoxin receptors or for translocation through the endosomal membrane in the heavy chain, but may contain one or more mutations comprising a deletion of one or more amino acids of the corresponding clostridial neurotoxin, an addition of one or more amino acids of the corresponding clostridial neurotoxin, and/or a substitution of one or more amino acids of the corresponding clostridial neurotoxin. Particularly, said deleted, added and/or substituted amino acids are consecutive amino acids. According to the teaching of the present invention, any number of amino acids may be added, deleted, and/or substituted, as long as the functional variant remains biologically active. For example, 1, 2, 3, 4, 5, up to 10, up to 15, up to 25, up to 50, up to 100, up to 200, up to 400, up to 500 amino acids or even more amino acids may be added, deleted, and/or substituted. Accordingly, a functional variant of the neurotoxin may be a biologically active fragment of a naturally occurring neurotoxin. This neurotoxin fragment may contain an N-terminal, C-terminal, and/or one or more internal deletion(s).

In another embodiment, the functional variant of a clostridial neurotoxin additionally comprises a signal peptide. Usually, said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has in its clostridium neurotoxin part a sequence identity of at least about 40%, at least about 50%, at least about 60%, at least about 70% or most particularly at least about 80%, and a sequence homology of at least about 60%, at least about 70%, at least about 80%, at least about 90%, or most particularly at least about 95% to the corresponding part in the parental clostridial neurotoxin. Methods and algorithms for determining sequence identity and/or homology, including the comparison of variants having deletions, additions, and/or substitutions relative to a parental sequence, are well known to the practitioner of ordinary skill in the art. On the DNA level, the nucleic acid sequences encoding the functional homologue and the parental clostridial neurotoxin may differ to a larger extent due to the degeneracy of the genetic code. It is known that the usage of codons is different between prokaryotic and eukaryotic organisms. Thus, when expressing a prokaryotic protein such as a clostridial neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a neurotoxin that is a chemically, enzymatically, or genetically modified derivative of a corresponding clostridial neurotoxin, including chemically or genetically modified neurotoxin from C. botulinum, particularly of C. botulinum neurotoxin serotype A, C or E. A chemically modified derivative may be one that is modified by pyruvation, phosphorylation, sulfatation, lipidation, pegylation, glycosylation and/or the chemical addition of an amino acid or a polypeptide comprising between 2 and about 100 amino acids, including modification occurring in the eukaryotic host cell used for expressing the derivative. An enzymatically modified derivative is one that is modified by the activity of enzymes, such as endo- or exoproteolytic enzymes, including modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said clostridial neurotoxin. Methods for designing and constructing such chemically or genetically modified derivatives and for testing of such variants for functionality are well known to anyone of ordinary skill in the art.

In particular embodiments, said recombinant clostridial neurotoxin has the amino acid sequence as found in any one of the sequences in Table 2 (SEQ ID NOs: 33 to 36).

The recombinant clostridial neurotoxins of the present invention shows increased membrane localization in vivo relative to an identical clostridial neurotoxin without the C2 domain.

In the context of the present invention, the term “increased/enhanced membrane localisation” means that the portion of recombinant neurotoxin showing membrane localisation is more than about 1.5-fold, particularly more than about 2-fold increased relative to the identical neurotoxin without the C2 domain as determined by confocal microscopy.

In the context of the present invention, the term “about” or “approximately” means within 20%, alternatively within 10%, including within 5% of a given value or range. Alternatively, especially in biological systems, the term “about” means within about a log (i.e. an order of magnitude), including within a factor of two of a given value.

In particular embodiments, said recombinant clostridial neurotoxin shows increased duration of effect relative to an identical clostridial neurotoxin without the C2 domain.

In particular embodiments, the C- or N-terminal fusion of a C2 domain to the clostridial neurotoxin light chain increases the membrane affinity of the clostridial neurotoxin light chain, resulting in the membrane association of the clostridial neurotoxin. Membrane binding of the clostridial neurotoxin prevents cytosolic degradation of the neurotoxin, thereby slowing down removal of the neurotoxin out of the neuronal cell. The catalytically active clostridial neurotoxin light chain is therefore longer available in the neuronal cell, causing increased duration of effect.

In the context of the present invention, the term “increased duration of effect” or “increased duration of action” refers to a longer lasting denervation mediated by a clostridial neurotoxin of the present invention. For example, as disclosed herein, administration of a disulfide-linked di-chain clostridial neurotoxin comprising a C2 domain results in localized paralysis for a longer period of time relative to administration of an identical disulfide-linked di-chain clostridial neurotoxin without the C2 domain.

In the context of the present invention, the term “increased duration of effect/action” is defined as a more than about 20%, particularly more than about 50%, more particularly more than about 90% increased duration of effect of the recombinant neurotoxin of the present invention relative to the identical neurotoxin without the C2 domain.

In the context of the present invention the term “chemodenervation” refers to denervation resulting from administration of a chemodenervating agent, for example a neurotoxin.

In the context of the present invention, the term “localized denervation” or “localized paralysis” refers to denervation of a particular anatomical region, usually a muscle or a group of anatomically and/or physiologically related muscles, which results from administration of a chemodenervating agent, for example a neurotoxin, to the particular anatomical region.

In particular embodiments, the increased duration of effect is due to an increased biological half-life.

In the context of the present invention, the term “biological half-life” specifies the lifespan of a protein, for example of a clostridial neurotoxin, in vivo. In the context of the present invention, the term “biological half-life” refers to the period of time, by which half of a protein pool is degraded in vivo. For example it refers to the period of time, by which half of the amount of clostridial neurotoxin of one administered dosage is degraded.

In the context of the present invention, the term “increased biological half-life” is defined as a more than about 20%, particularly more than about 50%, more particularly more than about 90% increased biological half-life of the recombinant neurotoxin of the present invention relative to the identical neurotoxin without the C2 domain.

In particular embodiments, the recombinant clostridial neurotoxin is for the use in the treatment of a disease requiring improved chemodenervation, wherein the recombinant clostridial neurotoxin causes longer lasting denervation relative to an identical clostridial neurotoxin without the C2 domain.

In particular other embodiments, the recombinant clostridial neurotoxin is for use in the treatment of (a) patients showing an immune reaction against BoNT/A, or (b) headache or epilepsy, wherein the recombinant clostridial neurotoxin is of serotype E.

In another aspect, the present invention relates to a pharmaceutical composition comprising the recombinant clostridial neurotoxin of the present invention.

In yet another aspect, the present invention relates to the use of the composition of the present invention for cosmetic treatment.

In particular embodiments, the recombinant clostridial neurotoxin of the present invention or the pharmaceutical composition of the present invention is for use in the treatment of a disease or condition taken from the list of: cervical dystonia (spasmodic torticollis), blepharospasm, severe primary axillary hyperhidrosis, achalasia, lower back pain, benign prostate hypertrophy, chronic focal painful neuropathies, migraine and other headache disorders, and cosmetic or aesthetic applications.

Additional indications where treatment with Botulinum neurotoxins is currently under investigation and where the pharmaceutical composition of the present invention may be used, include pediatric incontinence, incontinence due to overactive bladder, and incontinence due to neurogenic bladder, anal fissure, spastic disorders associated with injury or disease of the central nervous system including trauma, stroke, multiple sclerosis, Parkinson's disease, or cerebral palsy, focal dystonias affecting the limbs, face, jaw or vocal cords, temporomandibular joint (TMJ) pain disorders, diabetic neuropathy, wound healing, excessive salivation, vocal cord dysfunction, reduction of the Masseter muscle for decreasing the size of the lower jaw, treatment and prevention of chronic headache and chronic musculoskeletal pain, treatment of snoring noise, assistance in weight loss by increasing the gastric emptying time.

Most recently, clostridial neurotoxins have been evaluated for the treatment of other new indications, for example painful keloid, diabetic neuropathic pain, refractory knee pain, trigeminal neuralgia trigger-zone application to control pain, scarring after cleft-lip surgery, cancer and depression.

In another aspect, the present invention relates to a method for the generation of the recombinant clostridial neurotoxin of the present invention, comprising the step of obtaining a recombinant nucleic acid sequence encoding a recombinant single-chain precursor clostridial neurotoxin by the insertion of a nucleic acid sequence encoding said C2 domain into a nucleic acid sequence encoding a parental clostridial neurotoxin.

In the context of the present invention, the term “recombinant nucleic acid sequence” refers to a nucleic acid, which has been generated by joining genetic material from two different sources.

In the context of the present invention, the term “single-chain precursor clostridial neurotoxin” refers to a single-chain precursor for a disulfide-linked di-chain clostridial neurotoxin, comprising a functionally active clostridial neurotoxin light chain, a functionally active neurotoxin heavy chain, and a loop region linking the C-terminus of the light chain with the N-terminus of the heavy chain.

In the context of the present invention, the term “recombinant single-chain precursor clostridial neurotoxin” refers to a single-chain precursor clostridial neurotoxin comprising a heterologous C2 domain, i.e. a C2 domain from a species other than Clostridium botulinum.

In particular embodiments, the recombinant single-chain precursor clostridial neurotoxin comprises a protease cleavage site in said loop region.

Single-chain precursor clostridial neurotoxins have to be proteolytically cleaved to obtain the final biologically active clostridial neurotoxins. Proteolytic cleavage may either occur during heterologous expression by host cell enzymes, or by adding proteolytic enzymes to the raw protein material isolated after heterologous expression. Naturally occurring clostridial neurotoxins usually contain one or more cleavage signals for proteases which post-translationally cleave the single-chain precursor molecule, so that the final di- or multimeric complex can form. At present, clostridial neurotoxins are still predominantly produced by fermentation processes using appropriate Clostridium strains. During the fermentation process, the single-chain precursors are proteolytically cleaved by an unknown clostridial protease to obtain the biologically active di-chain clostridial neurotoxin. In cases, where the single-chain precursor molecule is the precursor of a protease, autocatalytic cleavage may occur. Alternatively, the protease can be a separate non-clostridial enzyme expressed in the same cell. WO 2006/076902 describes the proteolytic cleavage of a recombinant clostridial neurotoxin single-chain precursor at a heterologous recognition and cleavage site by incubation of the E. coli host cell lysate. The proteolytic cleavage is carried out by an unknown E. coli protease. In certain applications of recombinant expression, modified protease cleavage sites have been introduced recombinantly into the interchain region between the light and heavy chain of clostridial toxins, e.g. protease cleavage sites for human thrombin or non-human proteases (see WO 01/14570).

In particular embodiments, the protease cleavage site is a site that is cleaved by a protease selected from the list of: a protease selected from the list of: thrombin, trypsin, enterokinase, factor 1Xa, plant papain, insect papain, crustacean papain, enterokinase, human rhinovirus 3C protease, human enterovirus 3C protease, tobacco etch virus protease, Tobacco Vein Mottling Virus, subtilisin and caspase 3.

In a particular embodiment, the recombinant single-chain precursor clostridial neurotoxin further comprises a binding tag, particularly selected from the group comprising: glutathione-S-transferase (GST), maltose binding protein (MBP), a His-tag, a StrepTag, or a FLAG-tag.

In the context of the present invention, the term “parental clostridial neurotoxin” refers to an initial clostridial neurotoxin without a heterologous C2 domain, selected from a natural clostridial neurotoxin, a functional variant of a clostridial neurotoxin or a chimeric clostridial neurotoxin, wherein the clostridial neurotoxin light chain and heavy chain are from different clostridial neurotoxin serotypes.

In particular embodiments, the method for the generation of the recombinant clostridial neurotoxin of the present invention further comprises the step of heterologously expressing said recombinant nucleic acid sequence in a host cell, particularly in a bacterial host cell, more particularly in an E. coli host cell.

In certain embodiments, the E. coli cells are selected from E. coli XL1-Blue, Nova Blue, TOP10, XL10-Gold, BL21, and K12.

In particular embodiments, the method for the generation of the recombinant clostridial neurotoxin of the present invention additionally comprises at least one of the steps of (i) generating a disulfide-linked di-chain recombinant clostridial neurotoxin comprising a C2 domain by causing or allowing contacting of said recombinant single-chain precursor clostridial neurotoxin with an endoprotease and (ii) purification of said recombinant single-chain precursor clostridial neurotoxin or said disulfide-linked di-chain recombinant clostridial neurotoxin by chromatography.

In particular embodiments, the recombinant single-chain precursor clostridial neurotoxin, or the recombinant disulfide-linked di-chain clostridial neurotoxin, is purified after expression, or in the case of the recombinant disulfide-linked di-chain clostridial neurotoxin, after the cleavage reaction. In particular such embodiments, the protein is purified by chromatography, particularly by immunoaffinity chromatography, or by chromatography on an ion exchange matrix, a hydrophobic interaction matrix, or a multimodal chromatography matrix, particularly a strong ion exchange matrix, more particularly a strong cation exchange matrix.

In the context of the present invention, the term “causing . . . contacting of said recombinant single-chain precursor clostridial neurotoxin . . . with an endoprotease” refers to an active and/or direct step of bringing said neurotoxin and said endoprotease in contact, whereas the term “allowing contacting of a recombinant single-chain precursor clostridial neurotoxin . . . with an endoprotease” refers to an indirect step of establishing conditions in such a way that said neurotoxin and said endoprotease are getting in contact to each other.

In the context of the present invention, the term “endoprotease” refers to a protease that breaks peptide bonds of non-terminal amino acids (i.e. within the polypeptide chain). As they do not attack terminal amino acids, endoproteases cannot break down peptides into monomers.

In particular embodiments, cleavage of the recombinant single-chain precursor clostridial neurotoxin is near-complete.

In the context of the present invention, the term “near-complete” is defined as more than about 95% cleavage, particularly more than about 97.5%, more particularly more than about 99% as determined by SDS-PAGE and subsequent Western Blot or reversed phase chromatography.

In particular embodiments, cleavage of the recombinant single-chain precursor clostridial neurotoxin occurs at a heterologous cleavage signal located in the loop region of the recombinant precursor clostridial neurotoxin.

In particular embodiments, the cleavage reaction is performed with crude host cell lysates containing said single-chain precursor protein.

In other particular embodiments, the single-chain precursor protein is purified or partially purified, particularly by a first chromatographic enrichment step, prior to the cleavage reaction.

In the context of the present invention, the term “purified” relates to more than about 90% purity. In the context of the present invention, the term “partially purified” relates to purity of less than about 90% and an enrichment of more than about two fold.

In another aspect, the present invention relates to a recombinant single-chain precursor clostridial neurotoxin comprising a C2 domain.

In particular embodiments, said C2 domain is a C2 domain present in a protein listed in Table 5. In particular other embodiments, said C2 domain is a human C2 domain, particularly a human C2 domain present in a human protein listed in Table 6, particularly in Table 6A, particularly a human C2 domain present in a human protein selected from the list of: ABR; BAIP3; BCR; C2CD3; C2D1A; C2D1B; CAN5; CANE; CAPS1; CAPS2; CPNE1; CPNE2; CPNE3; CPNE4; CPNE5; CPNE6; CPNE7; CPNE8; CPNE9; CUO25; DAB2P; DOC2A; DOC2B; DYSF; ESYT1; ESYT2; ESYT3; FR1L5; FTM; HECW1; HECW2; ITCH; ITSN1; ITSN2; KPCA; KPCB; KPCE; KPCG; KPCL; MCTP1; MCTP2; MYOF; NEDD4; NED4L; NGAP; OTOF; P3C2A; P3C2B; P3C2G; PA24A; PA24B; PA24D; PA24E; PA24F; POLO; PERF; PLCB1; PLCB2; PLCB3; PLCB4; PLCD1; PLCD3; PLCD4; PLCE1; PLCG1; PLCG2; PLCH1; PLCH2; PLCL1; PLCL2; PLCZ1; RASA1; RASA2; RASA3; RASL1; RASL2; RFIP1; RFIP2; RFIP5; RGS3; RIMS1; RIMS2; RIMS3; RIMS4; RP3A; RPGR1; SMUF1; SMUF2; SY14L; SYGP1; SYT1; SYT2; SYT3; SYT4; SYT5; SYT6; SYT7; SYT8; SYT9; SYT10; SYT11; SYT12; SYT13; SYT14; SYT15; SYT16; SYT17; SYTL1; SYTL2; SYTL3; SYTL4; SYTL5; TAC2N; TOLIP; UN13A; UN13B; UN13C; UN13D; WWC2; WWP1; and WWP2. In particular embodiments, the C2 domain is a human C2 domain present in a human protein selected from the list of: DOC2A; DOC2B; DYSF; ESYT1; ESYT2; ESYT3; FR1L5; KPCA; KPCB; KPCG; MYOF; NED4L; PLCD1; PLCD3; PLCZ1; RFIP1; RFIP2; RFIP5; RP3A; SYT1; SYT2; SYT3; SYT4; SYT5; SYT6; SYT7; SYT9; SYT10; and SYTL1. In particular embodiments, the C2 domain is a human C2 domain present in a human protein selected from the list of: RP3A, ESYT1, ESYT2, and ESYT3.

In particular embodiments, said C2 domain has the amino acid sequence of one of the C2 domains listed in Table 1 (SEQ ID NOs: 1 to 32). More particularly said C2 domain is selected from SEQ ID NOs: 1 to 2 and SEQ ID NOs: 27 to 29.

In particular embodiments, said C2 domain is located at (i) the N-terminus of the light chain of the clostridial neurotoxin, or (ii) at the C-terminus of the light chain of the clostridial neurotoxin.

In particular embodiments, said clostridial neurotoxin is selected from (i) a Clostridium botulinum neurotoxin serotype A, B, C, D, E, F, and G, particularly Clostridium botulinum neurotoxin serotype A, C and E, or (ii) from a functional variant of a Clostridium botulinum neurotoxin of (i), or (iii) from a chimeric Clostridium botulinum neurotoxin, wherein the clostridial neurotoxin light chain and heavy chain are from different clostridial neurotoxin serotypes.

In particular embodiments, said recombinant single-chain precursor clostridial neurotoxin has the amino acid sequence as found in any one of the sequences in Table 2 (SEQ ID NOs: 33 to 36).

In another aspect, the present invention relates to a nucleic acid sequence encoding the recombinant single-chain precursor clostridial neurotoxin of the present invention, particularly a nucleic acid sequence comprising a C2 domain-coding nucleic acid sequence selected from the group of nucleic acid sequences of Table 3 (SEQ ID NOs: 37 to 68), particularly wherein said nucleic acid has the sequence as found in Table 4 (SEQ ID NOs: 69 to 72).

In another aspect, the present invention relates to a method for obtaining the nucleic acid sequence of the present invention, comprising the step of inserting a nucleic acid sequence encoding a C2 domain into a nucleic acid sequence encoding a parental clostridial neurotoxin.

In another aspect, the present invention relates to a vector comprising the nucleic acid sequence of the present invention, or the nucleic acid sequence obtainable by the method of the present invention.

In another aspect, the present invention relates to a recombinant host cell comprising the nucleic acid sequence of the present invention, the nucleic acid sequence obtainable by the method of the present invention, or the vector of the present invention.

In certain embodiments, the recombinant host cells are selected from E. coli XL1-Blue, Nova Blue, TOP10, XL10-Gold, BL21, and K12.

In another aspect, the present invention relates to a method for producing the recombinant single-chain precursor clostridial neurotoxin of the present invention, comprising the step of expressing the nucleic acid sequence of the present invention, or the nucleic acid sequence obtainable by the method of the present invention, or the vector of the present invention in a recombinant host cell, or cultivating the recombinant host cell of the present invention under conditions that result in the expression of said nucleic acid sequence.

### EXAMPLES

### Example 1

### Generation of a Botulinum Toxin Construct Comprising an N-Terminal C2 Domain

A DNA sequence coding for a C2 domain is attached to a DNA sequence coding for botulinum toxin type A comprised in an expression vector for E. coli by means of gene synthesis and subcloning. The generated construct is transformed into the E. coli expression strain BL21 and the modified botulinum toxin is heterologously expressed. Purification of the toxin from E. coli cell lysates is performed by immunoaffinity chromatography (His-Tag), ion exchange chromatography, and gel filtration.

Table 4 shows the sequences of two exemplary constructs with C2 domains added N-terminally (SEQ ID NOs: 69 and 70). The constructs comprise a sequence encoding a thrombin cleavage site in the loop region.

### Example 2

### Generation of a Botulinum Toxin Construct Comprising a C2 Domain at the C-Terminus of the Light Chain

A DNA sequence coding for a C2 domain is inserted into the DNA sequence coding for a botulinum toxin type A between the DNA segments coding for the light and the heavy chain by means of gene synthesis and sub cloning. This construct is generated in an expression vector for E. coli, transformed into the E. coli expression strain BL21 and the modified botulinum toxin is heterologously expressed. The purification of the toxin from E. coli cell lysates is performed by immunoaffinity chromatography (His-Tag), ion exchange chromatography, and gel filtration.

TABLE 1
Human C2 Domains

| Human | SEQ | |
|---|---|---|
| Protein | ID NO: | Protein Sequence |
| RP3A (C2 | 1 | SLQCTIIKAKGLKPMDSNGLADPYVKLH |
| domain 1) | | LLPGASKSNKLRTKTLRNTRNPIWNETL |
| | | VYHGITDEDMQRKTLRISVCDEDKFGHN |
| | | EFIGETRFSLKKLKPNQRKNFN |
| RP3A (C2 | 2 | GLIVGIIRCVHLAAMDANGYSDPFVKLW |
| domain 2) | | LKPDMGKKAKHKTQIKKKTLNPEFNEEF |
| | | FYDIKHSDLAKKSLDISVWDYDIGKSND |
| | | YIGGCQLGISAKGERLKHWYECLKNKDK |
| | | KIE |
| DOC2A | 3 | TLHCSILRAKGLKPMDFNGLADPYVKLH |
| | | LLPGACKANKLKTKTQRNTLNPVWNEDL |
| | | TYSGITDDDITHKVLRIAVCDEDKLSHN |
| | | EFIGEIRVPLRRLKPSQKKHFN |
| DOC2B | 4 | ALHCTITKAKGLKPMDHNGLADPYVKLH |
| | | LLPGASKANKLRTKTLRNTLNPTWNETL |
| | | TYYGITDEDMIRKTLRISVCDEDKFRHN |
| | | EFIGETRVPLKKLKPNHTKTFS |
| SYTL1 | 5 | ELRVHVIQCQGLAAARRRRSDPYVKSYL |
| | | LPDKQSKRKTAVKKRNLNPVFNETLRYS |
| | | VPQAELQGRVLSLSVWHRESLGRNIFLG |
| | | EVEVPLDTWDWGSEPTWL |
| NED4L | 6 | ILRVKVVSGIDLAKKDIFGASDPYVKLS |
| | | LYVADENRELALVQTKTIKKTLNPKWNE |
| | | EFYFRVNPSNHRLLFEVFDENRLTRDDF |
| | | LGQVDVPLSHLPTEDPTMER |
| KPCA | 7 | KLHVTVRDAKNLIPMDPNGLSDPYVKLK |
| | | LIPDPKNESKQKTKTIRSTLNPQWNESF |
| | | TFKLKPSDKDRRLSVEIWDWDRTTRNDF |
| | | MGSLSFGVSELMKMPASGWY |
| KPCB | 8 | VLIVLVRDAKNLVPMDPNGLSDPYVKLK |
| | | LIPDPKSESKQKTKTIKCSLNPEWNETF |
| | | RFQLKESDKDRRLSVEIWDWDLTSRNDF |
| | | MGSLSFGISELQKASVDGWF |
| KPCG | 9 | EIHVTVGEARNLIPMDPNGLSDPYVKLK |
| | | LIPDPRNLTKQKTRTVKATLNPVWNETF |
| | | VFNLKPGDVERRLSVEVWDWDRTSRNDF |
| | | MGAMSFGVSELLKAPVDGWY |
| PLCD1 | 10 | RLNIRVISGQQLPKVNKNKNSIVDPKVT |
| | | VEIHGVSRDVASRQTAVITNNGFNPWWD |
| | | TEFAFEVVVPDLALIRFLVEDYDASSKN |
| | | DFIGQSTIPLNSLKQGYRHVHL |
| PLCD3 | 11 | TLSIQVLTAQQLPKLNAEKPHSIVDPLV |
| | | RIEIHGVPADCARQETDYVLNNGFNPRW |
| | | GQTLQFQLRAPELALVRFVVEDYDATSP |
| | | NDFVGQFTLPLSSLKQGYRHIHL |
| PLCZ1 | 12 | TLTIRLISGIQLPLTHSSSNKGDSLVII |
| | | EVFGVPNDQMKQQTRVIKKNAFSPRWNE |
| | | TFTFIIHVPELALIRFVVEGQGLIAGNE |
| | | FLGQYTLPLLCMNKGYRRIPL |
| PTEN | 13 | YRPVALLFHKMMFETIPMFSGGTCNPQF |
| | | VVCQLKVKIYSSNSGPTRREDKFMYFEF |
| | | PQPLPVCGDIKVEFFHKQNKMLKKDKMF |
| | | HFWVNTFFIPGPEETSEKVENGSLCDQE |
| | | IDSICSIERADNDKEYLVLTLTKNDLDK |
| | | ANKDKANRYFSPNFKVKLYFTK |
| RFIP1 | 14 | HVQVTVLQARGLRAKGPGGTSDAYAVIQ |
| | | VGKEKYATSVSERSLGAPVWREEATFEL |
| | | PSLLSSGPAAAATLQLTVLHRALLGLDK |
| | | FLGRAEVDLRDLHRDQGRRKT |
| RFIP5 | 15 | HVQVTVLRARGLRGKSSGAGSTSDAYTV |
| | | IQVGREKYSTSVVEKTHGCPEWREECSF |
| | | ELPPGALDGLLRAQEADAGPAPWAASSA |
| | | AACELVLTTMHRSLIGVDKFLGQATVAL |
| | | DEVFGAGRAQHT |
| RFIP2 | 16 | HVQVTVLQAKDLKPKGKSGTNDTYTIIQ |
| | | LGKEKYSTSVAEKTLEPVWKEEASFELP |
| | | GLLIQGSPEKYILFLIVMHRSLVGLDKF |
| | | LGQVAINLNDIFEDKQRRKT |
| SYT1 (C2 | 17 | QLLVGIIQAAELPALDMGGTSDPYVKVF |
| domain 1) | | LLPDKKKKFETKVHRKTLNPVFNEQFTF |
| | | KVPYSELGGKTLVMAVYDFDRFSKHDII |
| | | GEFKVPMNTVDFGHVTEEW |
| SYT1 (C2 | 18 | KLTVVILEAKNLKKMDVGGLSDPYVKIH |
| domain 2) | | LMQNGKRLKKKKTTIKKNTLNPYYNESF |
| | | SFEVPFEQIQKVQVVVTVLDYDKIGKND |
| | | AIGKVFVGYNSTGAELRHWSDMLANPRR |
| | | PIA |
| SYT2 | 19 | QLTVGVLQAAELPALDMGGTSDPYVKVF |
| | | LLPDKKKKYETKVHRKTLNPAFNETFTF |
| | | KVPYQELGGKTLVMAIYDFDRFSKHDII |
| | | GEVKVPMNTVDLGQPIEEW |
| SYT3 | 20 | QLVVRILQALDLPAKDSNGFSDPYVKIY |
| | | LLPDRKKKFQTKVHRKTLNPVFNETFQF |
| | | SVPLAELAQRKLHFSVYDFDRFSRHDLI |
| | | GQVVLDNLLELAEQPPDRPL |
| SYT4 | 21 | AFVVNIKEARGLPAMDEQSMTSDPYIKM |
| | | TILPEKKHKVKTRVLRKTLDPAFDETFT |
| | | FYGIPYTQIQELALHFTILSFDRFSRDD |
| | | IIGEVLIPLSGIELSEGKMLM |
| SYT5 | 22 | QLLVGILQAMGLAALDLGGSSDPYVRVY |
| | | LLPDKRRRYETKVHRQTLNPHFGETFAF |
| | | KVPYVELGGRVLVMAVYDFDRFSRNDAI |
| | | GEVRVPMSSVDLGRPVQAW |
| SYT6 | 23 | TLIVRILKAFDLPAKDFCGSSDPYVKIY |
| | | LLPDRKCKLQTRVHRKTLNPTFDENFHF |
| | | PVPYEELADRKLHLSVFDFDRFSRHDMI |
| | | GEVILDNLFEASDLSRETSIW |
| SYT7 | 24 | TLTVKIMKAQELPAKDFSGTSDPFVKIY |
| | | LLPDKKHKLETKVKRKNLNPHWNETFLF |
| | | EGFPYEKVVQRILYLQVLDYDRFSRNDP |
| | | IGEVSIPLNKVDLTQMQTFW |
| SYT9 | 25 | QLIVKIHKAVNLPAKDFSGTSDPYVKIY |
| | | LLPDRKTKHQTKVHRKTLNPVFDEVFLF |
| | | PVPYNDLEARKLHFSVYDFDRFSRHDLI |
| | | GQVVVDHFLDLADFPRECIL |
| SYT10 | 26 | LLVVKIIKALDLPAKDFTGTSDPYVKMY |
| | | LLPDRKKKFQTRVHRKTLNPLFDETFQF |
| | | PVAYDQLSNRKLHFSVYDFDRFSRHDMI |
| | | GEVILDNLFEVSDLSREATV |
| ESYT1 | 27 | KLVSIVHGCRSLRQNGRDPPDPYVSLLL |
| | | LPDKNRGTKRRTSQKKRTLSPEFNERFE |
| | | WELPLDEAQRRKLDVSVKSNSSFMSRER |
| | | ELLGKVQLDLAETDLSQGVARW |
| ESYT2 | 28 | LGQIQLTIRHSSQRNKLIVVVHACRNLI |
| | | AFSEDGSDPYVRMYLLPDKRRSGRRKTH |
| | | VSKKTLNPVFDQSFDFSVSLPEVQRRTL |
| | | DVAVKNSGGFLSKDKGLLGKVLVALASE |
| | | ELAKGWTQWYDLTEDGT |
| ESYT3 | 29 | CLSVLINGCRNLTPCTSSGADPYVRVYL |
| | | LPERKWACRKKTSVKRKTLEPLFDETFE |
| | | FFVPMEEVKKRSLDVAVKNSRPLGSHRR |
| | | KELGKVLIDLSKEDLIKGFSQWYE |
| DYSF | 30 | MLCCLLVRASNLPSAKKDRRSDPVASLT |
| | | FRGVKKRTKVIKNSVNPVWNEGFEWDLK |
| | | GIPLDQGSELHVVVKDHETMGRNRFLGE |
| | | AKVPLREVLATPSLSAS |
| MYOF | 31 | MLRVIVESASNIPKTKFGKPDPIVSVIF |
| | | KDEKKKTKKVDNELNPVWNEILEFDLRG |
| | | IPLDFSSSLGIIVKDFETIGQNKLIGTA |
| | | TVALKDLTGDQSRSLP |
| FR1L5 | 32 | QVRVKVFEARQLMGNNIKPVVKVSIAGQ |
| | | QHQTRIKMGNNPFFNEIFFQNFHEVPAK |
| | | FFDETILIQTDIGFIYHSPGHTLLRKWL |
| | | GLCQPNNPGSG |

TABLE 2
C2 Domain-Containing Constructs

| | SEQ | C2 | |
|---|---|---|---|
| Insertion | ID NO: | domain: | BoNT/A with C2 Domain |
| N-terminal | 33 | RP3A (C2 | MSLQCTIIKAKGLKPMDSNGLADPYVKLHLLPGASKSNKLRTKTLRNTRNP |
| | | domain 1) | IWNETLVYHGITDEDMQRKTLRISVCDEDKFGHNEFIGETRFSLKKLKPNQ |
| | | | RKNFNPFVNKQFNYKDPVNGVDIAYIKIPNAGQMQPVKAFKIHNKIWVIPE |
| | | | RDTFTNPEEGDLNPPPEAKQVPVSYYDSTYLSTDNEKDNYLKGVTKLFERI |
| | | | YSTDLGRMLLTSIVRGIPFWGGSTIDTELKVIDTNCINVIQPDGSYRSEEL |
| | | | NLVIIGPSADIIQFECKSFGHEVLNLTRNGYGSTQYIRFSPDFTFGFEESL |
| | | | EVDTNPLLGAGKFATDPAVTLAHELIHAGHRLYGIAINPNRVFKVNTNAYY |
| | | | EMSGLEVSFEELRTFGGHDAKFIDSLQENEFRLYYYNKFKDIASTLNKAKS |
| | | | IVGTTASLQYMKNVFKEKYLLSEDTSGKFSVDKLKFDKLYKMLTEIYTEDN |
| | | | FVKFFKVLNRKTYLNFDKAVFKINIVPKVNYTIYDGFNLRNTNLAANFNGQ |
| | | | NTEINNMNFTKLKNFTGLFEFYKLLCVRGIITSKAGAGKSLVPRGSAGAGA |
| | | | LNDLCIKVNNWDLFFSPSEDNFTNDLNKGEEITSDTNIEAAEENISLDLIQ |
| | | | QYYLTFNFDNEPENISIENLSSDIIGQLELMPNIERFPNGKKYELDKYTMF |
| | | | HYLRAQEFEHGKSRIALTNSVNEALLNPSRVYTFFSSDYVKKVNKATEAAM |
| | | | FLGWVEQLVYDFTDETSEVSTTDKIADITIIIPYIGPALNIGNMLYKDDFV |
| | | | GALIFSGAVILLEFIPEIAIPVLGTFALVSYIANKVLTVQTIDNALSKRNE |
| | | | KWDEVYKYIVTNWLAKVNTQIDLIRKKMKEALENQAEATKAIINYQYNQYT |
| | | | EEEKNNINFNIDDLSSKLNESINKAMININKFLNQCSVSYLMNSMIPYGVK |
| | | | RLEDFDASLKDALLKYIYDNRGTLIGQVDRLKDKVNNTLSTDIPFQLSKYV |
| | | | DNQRLLSTFTEYIKNIINTSILNLRYESNHLIDLSRYASKINIGSKVNFDP |
| | | | IDKNQIQLFNLESSKIEVILKNAIVYNSMYENFSTSFWIRIPKYFNSISLN |
| | | | NEYTIINCMENNSGWKVSLNYGEIIWTLQDTQEIKQRVVFKYSQMINISDY |
| | | | INRWIFVTITNNRLNNSKIYINGRLIDQKPISNLGNIHASNNIMFKLDGCR |
| | | | DTHRYIWIKYFNLFDKELNEKEIKDLYDNQSNSGILKDFWGDYLQYDKPYY |
| | | | MLNLYDPNKYVDVNNVGIRGYMYLKGPRGSVMTTNIYLNSSLYRGTKFIIK |
| | | | KYASGNKDNIVRNNDRVYINVVVKNKEYRLATNASQAGVEKILSALEIPDV |
| | | | GNLSQVVVMKSKNDQGITNKCKMNLQDNNGNDIGFIGFHQFNNIAKLVASN |
| | | | WYNRQIERSSRTLGCSWEFIPVDDGWGERPL- |
| N-terminal | 34 | ESYT2 | MLGQIQLTIRHSSQRNKLIVVVHACRNLIAFSEDGSDPYVRMYLLPDKRRS |
| | | | GRRKTHVSKKTLNPVFDQSFDFSVSLPEVQRRTLDVAVKNSGGFLSKDKGL |
| | | | LGKVLVALASEELAKGWTQWYDLTEDGTPFVNKQFNYKDPVNGVDIAYIKI |
| | | | PNAGQMQPVKAFKIHNKIWVIPERDTFTNPEEGDLNPPPEAKQVPVSYYDS |
| | | | TYLSTDNEKDNYLKGVTKLFERIYSTDLGRMLLTSIVRGIPFWGGSTIDTE |
| | | | LKVIDTNCINVIQPDGSYRSEELNLVIIGPSADIIQFECKSFGHEVLNLTR |
| | | | NGYGSTQYIRFSPDFTFGFEESLEVDTNPLLGAGKFATDPAVTLAHELIHA |
| | | | GHRLYGIAINPNRVFKVNTNAYYEMSGLEVSFEELRTFGGHDAKFIDSLQE |
| | | | NEFRLYYYNKFKDIASTLNKAKSIVGTTASLQYMKNVFKEKYLLSEDTSGK |
| | | | FSVDKLKFDKLYKMLTEIYTEDNFVKFFKVLNRKTYLNFDKAVFKINIVPK |
| | | | VNYTIYDGFNLRNTNLAANFNGQNTEINNMNFTKLKNFTGLFEFYKLLCVR |
| | | | GIITSKAGAGKSLVPRGSAGAGALNDLCIKVNNWDLFFSPSEDNFTNDLNK |
| | | | GEEITSDTNIEAAEENISLDLIQQYYLTFNFDNEPENISIENLSSDIIGQL |
| | | | ELMPNIERFPNGKKYELDKYTMFHYLRAQEFEHGKSRIALTNSVNEALLNP |
| | | | SRVYTFFSSDYVKKVNKATEAAMFLGWVEQLVYDFTDETSEVSTTDKIADI |
| | | | TIIIPYIGPALNIGNMLYKDDFVGALIFSGAVILLEFIPEIAIPVLGTFAL |
| | | | VSYIANKVLTVQTIDNALSKRNEKWDEVYKYIVTNWLAKVNTQIDLIRKKM |
| | | | KEALENQAEATKAIINYQYNQYTEEEKNNINFNIDDLSSKLNESINKAMIN |
| | | | INKFLNQCSVSYLMNSMIPYGVKRLEDFDASLKDALLKYIYDNRGTLIGQV |
| | | | DRLKDKVNNTLSTDIPFQLSKYVDNQRLLSTFTEYIKNIINTSILNLRYES |
| | | | NHLIDLSRYASKINIGSKVNFDPIDKNQIQLFNLESSKIEVILKNAIVYNS |
| | | | MYENFSTSFWIRIPKYFNSISLNNEYTIINCMENNSGWKVSLNYGEIIWTL |
| | | | QDTQEIKQRVVFKYSQMINISDYINRWIFVTITNNRLNNSKIYINGRLIDQ |
| | | | KPISNLGNIHASNNIMFKLDGCRDTHRYIWIKYFNLFDKELNEKEIKDLYD |
| | | | NQSNSGILKDFWGDYLQYDKPYYMLNLYDPNKYVDVNNVGIRGYMYLKGPR |
| | | | GSVMTTNIYLNSSLYRGTKFIIKKYASGNKDNIVRNNDRVYINVVVKNKEY |
| | | | RLATNASQAGVEKILSALEIPDVGNLSQVVVMKSKNDQGITNKCKMNLQDN |
| | | | NGNDIGFIGFHQFNNIAKLVASNWYNRQIERSSRTLGCSWEFIPVDDGWGE |
| | | | RPL- |
| Linker | 35 | RP3A (C2 | MPFVNKQFNYKDPVNGVDIAYIKIPNAGQMQPVKAFKIHNKIWVIPERDTF |
| region | | domain 1) | TNPEEGDLNPPPEAKQVPVSYYDSTYLSTDNEKDNYLKGVTKLFERIYSTD |
| | | | LGRMLLTSIVRGIPFWGGSTIDTELKVIDTNCINVIQPDGSYRSEELNLVI |
| | | | IGPSADIIQFECKSFGHEVLNLTRNGYGSTQYIRFSPDFTFGFEESLEVDT |
| | | | NPLLGAGKFATDPAVTLAHELIHAGHRLYGIAINPNRVFKVNTNAYYEMSG |
| | | | LEVSFEELRTFGGHDAKFIDSLQENEFRLYYYNKFKDIASTLNKAKSIVGT |
| | | | TASLQYMKNVFKEKYLLSEDTSGKFSVDKLKFDKLYKMLTEIYTEDNFVKF |
| | | | FKVLNRKTYLNFDKAVFKINIVPKVNYTIYDGFNLRNTNLAANFNGQNTEI |
| | | | NNMNFTKLKNFTGLFEFYKLLCVRGIITSLQCTIIKAKGLKPMDSNGLADP |
| | | | YVKLHLLPGASKSNKLRTKTLRNTRNPIWNETLVYHGITDEDMQRKTLRIS |
| | | | VCDEDKFGHNEFIGETRFSLKKLKPNQRKNFNSKAGAGKSLVPRGSAGAGA |
| | | | LNDLCIKVNNWDLFFSPSEDNFTNDLNKGEEITSDTNIEAAEENISLDLIQ |
| | | | QYYLTFNFDNEPENISIENLSSDIIGQLELMPNIERFPNGKKYELDKYTMF |
| | | | HYLRAQEFEHGKSRIALTNSVNEALLNPSRVYTFFSSDYVKKVNKATEAAM |
| | | | FLGWVEQLVYDFTDETSEVSTTDKIADITIIIPYIGPALNIGNMLYKDDFV |
| | | | GALIFSGAVILLEFIPEIAIPVLGTFALVSYIANKVLTVQTIDNALSKRNE |
| | | | KWDEVYKYIVTNWLAKVNTQIDLIRKKMKEALENQAEATKAIINYQYNQYT |
| | | | EEEKNNINFNIDDLSSKLNESINKAMININKFLNQCSVSYLMNSMIPYGVK |
| | | | RLEDFDASLKDALLKYIYDNRGTLIGQVDRLKDKVNNTLSTDIPFQLSKYV |
| | | | DNQRLLSTFTEYIKNIINTSILNLRYESNHLIDLSRYASKINIGSKVNFDP |
| | | | IDKNQIQLFNLESSKIEVILKNAIVYNSMYENFSTSFWIRIPKYFNSISLN |
| | | | NEYTIINCMENNSGWKVSLNYGEIIWTLQDTQEIKQRVVFKYSQMINISDY |
| | | | INRWIFVTITNNRLNNSKIYINGRLIDQKPISNLGNIHASNNIMFKLDGCR |
| | | | DTHRYIWIKYFNLFDKELNEKEIKDLYDNQSNSGILKDFWGDYLQYDKPYY |
| | | | MLNLYDPNKYVDVNNVGIRGYMYLKGPRGSVMTTNIYLNSSLYRGTKFIIK |
| | | | KYASGNKDNIVRNNDRVYINVVVKNKEYRLATNASQAGVEKILSALEIPDV |
| | | | GNLSQVVVMKSKNDQGITNKCKMNLQDNNGNDIGFIGFHQFNNIAKLVASN |
| | | | WYNRQIERSSRTLGCSWEFIPVDDGWGERPL- |
| Linker | 36 | ESYT2 | MPFVNKQFNYKDPVNGVDIAYIKIPNAGQMQPVKAFKIHNKIWVIPERDTF |
| region | | | TNPEEGDLNPPPEAKQVPVSYYDSTYLSTDNEKDNYLKGVTKLFERIYSTD |
| | | | LGRMLLTSIVRGIPFWGGSTIDTELKVIDTNCINVIQPDGSYRSEELNLVI |
| | | | IGPSADIIQFECKSFGHEVLNLTRNGYGSTQYIRFSPDFTFGFEESLEVDT |
| | | | NPLLGAGKFATDPAVTLAHELIHAGHRLYGIAINPNRVFKVNTNAYYEMSG |
| | | | LEVSFEELRTFGGHDAKFIDSLQENEFRLYYYNKFKDIASTLNKAKSIVGT |
| | | | TASLQYMKNVFKEKYLLSEDTSGKFSVDKLKFDKLYKMLTEIYTEDNFVKF |
| | | | FKVLNRKTYLNFDKAVFKINIVPKVNYTIYDGFNLRNTNLAANFNGQNTEI |
| | | | NNMNFTKLKNFTGLFEFYKLLCVRGIITLGQIQLTIRHSSQRNKLIVVVHA |
| | | | CRNLIAFSEDGSDPYVRMYLLPDKRRSGRRKTHVSKKTLNPVFDQSFDFSV |
| | | | SLPEVQRRTLDVAVKNSGGFLSKDKGLLGKVLVALASEELAKGWTQWYDLT |
| | | | EDGTSKAGAGKSLVPRGSAGAGALNDLCIKVNNWDLFFSPSEDNFTNDLNK |
| | | | GEEITSDTNIEAAEENISLDLIQQYYLTFNFDNEPENISIENLSSDIIGQL |
| | | | ELMPNIERFPNGKKYELDKYTMFHYLRAQEFEHGKSRIALTNSVNEALLNP |
| | | | SRVYTFFSSDYVKKVNKATEAAMFLGWVEQLVYDFTDETSEVSTTDKIADI |
| | | | TIIIPYIGPALNIGNMLYKDDFVGALIFSGAVILLEFIPEIAIPVLGTFAL |
| | | | VSYIANKVLTVQTIDNALSKRNEKWDEVYKYIVTNWLAKVNTQIDLIRKKM |
| | | | KEALENQAEATKAIINYQYNQYTEEEKNNINFNIDDLSSKLNESINKAMIN |
| | | | INKFLNQCSVSYLMNSMIPYGVKRLEDFDASLKDALLKYIYDNRGTLIGQV |
| | | | DRLKDKVNNTLSTDIPFQLSKYVDNQRLLSTFTEYIKNIINTSILNLRYES |
| | | | NHLIDLSRYASKINIGSKVNFDPIDKNQIQLFNLESSKIEVILKNAIVYNS |
| | | | MYENFSTSFWIRIPKYFNSISLNNEYTIINCMENNSGWKVSLNYGEIIWTL |
| | | | QDTQEIKQRVVFKYSQMINISDYINRWIFVTITNNRLNNSKIYINGRLIDQ |
| | | | KPISNLGNIHASNNIMFKLDGCRDTHRYIWIKYFNLFDKELNEKEIKDLYD |
| | | | NQSNSGILKDFWGDYLQYDKPYYMLNLYDPNKYVDVNNVGIRGYMYLKGPR |
| | | | GSVMTTNIYLNSSLYRGTKFIIKKYASGNKDNIVRNNDRVYINVVVKNKEY |
| | | | RLATNASQAGVEKILSALEIPDVGNLSQVVVMKSKNDQGITNKCKMNLQDN |
| | | | NGNDIGFIGFHQFNNIAKLVASNWYNRQIERSSRTLGCSWEFIPVDDGWGE |
| | | | RPL- |

TABLE 3
Human C2 Domain-coding Sequences

| Human | SEQ | |
|---|---|---|
| Protein | ID NO: | Protein Sequence |
| RP3A (C2 | 37 | TCCCTGCAGTGCACCATCATTAAGGCCAAGGGCCTGAAGCCCATGGATTCAAACGGCTTGGCTGATCCCTAC |
| domain 1) | | GTTAAGCTGCACCTCCTGCCGGGAGCCAGCAAGTCCAACAAGCTTCGTACAAAAACTCTGCGGAATACCCGG |
| | | AACCCCATCTGGAATGAGACCCTCGTGTATCACGGCATCACCGATGAGGACATGCAAAGGAAGACCCTCAGG |
| | | ATCTCCGTCTGTGATGAGGACAAATTTGGCCACAATGAATTTATTGGTGAGACCAGATTCTCCCTCAAGAAA |
| | | CTGAAGCCCAACCAGAGGAAGAATTTCAAC |
| RP3A (C2 | 38 | GGCCTCATTGTGGGCATCATACGCTGCGTGCACCTGGCTGCCATGGACGCTAATGGCTACTCAGACCCATTC |
| domain 2) | | GTCAAGCTCTGGCTGAAACCGGACATGGGAAAGAAGGCCAAACACAAGACTCAAATTAAAAAGAAAACCTTG |
| | | AATCCCGAATTCAATGAGGAGTTTTTCTATGACATCAAACACAGTGACCTGGCAAAGAAGTCACTGGACATT |
| | | TCAGTCTGGGACTATGACATCGGCAAGTCCAATGATTACATCGGAGGCTGCCAGCTGGGGATCTCTGCCAAG |
| | | GGAGAGCGCTTAAAACACTGGTACGAGTGTCTGAAAAATAAAGACAAGAAGATAGAG |
| DOC2A | 39 | ACTCTGCACTGTAGCATCCTCAGGGCCAAGGGCCTCAAGCCCATGGATTTCAATGGCCTCGCCGACCCCTAC |
| | | GTCAAGCTGCACTTGCTGCCTGGAGCCTGTAAGGCCAATAAGCTAAAAACGAAGACTCAGAGGAACACACTG |
| | | AATCCCGTGTGGAATGAGGACCTGACTTACAGCGGGATCACAGATGACGACATCACGCACAAGGTGCTCAGG |
| | | ATCGCCGTCTGTGATGAGGACAAGCTGAGTCACAATGAGTTTATTGGGGAGATCCGCGTGCCCCTCCGCCGC |
| | | CTCAAGCCTTCGCAGAAGAAGCATTTTAAC |
| DOC2B | 40 | GCCCTCCACTGCACCATCACCAAGGCCAAGGGCCTGAAGCCAATGGACCACAATGGGCTGGCAGACCCCTAC |
| | | GTCAAGCTGCACCTGCTGCCAGGAGCCAGTAAGGCAAATAAGCTCAGAACAAAAACTCTCCGTAACACTCTG |
| | | AACCCCACATGGAACGAGACCCTCACTTACTACGGGATCACAGATGAAGACATGATCCGCAAGACCCTGCGG |
| | | ATCTCTGTGTGTGACGAGGACAAATTCCGGCACAATGAGTTCATCGGGGAGACACGTGTGCCCCTGAAGAAG |
| | | CTGAAACCCAACCACACCAAGACCTTCAGC |
| SYTL1 | 41 | GAGCTGCGCGTGCACGTGATCCAGTGCCAGGGCCTGGCCGCCGCCCGGCGCCGCCGCTCGGACCCCTACGTC |
| | | AAAAGCTACCTCCTCCCGGATAAGCAGAGCAAGCGCAAGACGGCGGTGAAGAAACGGAATCTGAATCCGGTT |
| | | TTCAACGAGACTCTCCGGTACTCCGTCCCGCAGGCCGAGCTTCAGGGCCGCGTGCTGAGCCTGTCTGTGTGG |
| | | CACCGCGAAAGCCTGGGTCGCAACATCTTTCTGGGCGAAGTTGAAGTGCCCCTGGACACGTGGGACTGGGGC |
| | | TCTGAGCCCACCTGGCTC |
| NED4L | 42 | ATTCTCAGAGTAAAAGTTGTTTCTGGAATTGATCTCGCCAAAAAGGACATCTTTGGAGCCAGTGATCCGTAT |
| | | GTGAAACTTTCATTGTACGTAGCGGATGAGAATAGAGAACTTGCTTTGGTCCAGACAAAAACAATTAAAAAG |
| | | ACACTGAACCCAAAATGGAATGAAGAATTTTATTTCAGGGTAAACCCATCTAATCACAGACTCCTATTTGAA |
| | | GTATTTGACGAAAATAGACTGACACGAGACGACTTCCTGGGCCAGGTGGACGTGCCCCTTAGTCACCTTCCG |
| | | ACAGAAGATCCAACCATGGAGCGA |
| KPCA | 43 | AAGCTCCATGTCACAGTACGAGATGCAAAAAATCTAATCCCTATGGATCCAAACGGGCTTTCAGATCCTTAT |
| | | GTGAAGCTGAAACTTATTCCTGATCCCAAGAATGAAAGCAAGCAAAAAACCAAAACCATCCGCTCCACACTA |
| | | AATCCGCAGTGGAATGAGTCCTTTACATTCAAATTGAAACCTTCAGACAAAGACCGACGACTGTCTGTAGAA |
| | | ATCTGGGACTGGGATCGAACAACAAGGAATGACTTCATGGGATCCCTTTCCTTTGGAGTTTCGGAGCTGATG |
| | | AAGATGCCGGCCAGTGGATGGTAC |
| KPCB | 44 | GTCCTCATTGTCCTCGTAAGAGATGCTAAAAACCTTGTACCTATGGACCCCAATGGCCTGTCAGATCCCTAC |
| | | GTAAAACTGAAACTGATTCCCGATCCCAAAAGTGAGAGCAAACAGAAGACCAAAACCATCAAATGCTCCCTC |
| | | AACCCTGAGTGGAATGAGACATTTAGATTTCAGCTGAAAGAATCGGACAAAGACAGAAGACTGTCAGTAGAG |
| | | ATTTGGGATTGGGATTTGACCAGCAGGAATGACTTCATGGGATCTTTGTCCTTTGGGATTTCTGAACTTCAG |
| | | AAAGCCAGTGTTGATGGCTGGTTT |
| KPCG | 45 | GAGATCCACGTAACTGTTGGCGAGGCCCGTAACCTAATTCCTATGGACCCCAATGGTCTCTCTGATCCCTAT |
| | | GTGAAACTGAAGCTCATCCCAGACCCTCGGAACCTGACGAAACAGAAGACCCGAACGGTGAAAGCCACGCTA |
| | | AACCCTGTGTGGAATGAGACCTTTGTGTTCAACCTGAAGCCAGGGGATGTGGAGCGCCGGCTCAGCGTGGAG |
| | | GTGTGGGACTGGGACCGGACCTCCCGCAACGACTTCATGGGGGCCATGTCCTTTGGCGTCTCGGAGCTGCTC |
| | | AAGGCGCCCGTGGATGGCTGGTAC |
| PLCD1 | 46 | CGGCTCAACATCAGGGTCATTTCGGGGCAGCAGCTGCCAAAAGTCAACAAGAATAAGAATTCAATTGTGGAC |
| | | CCCAAAGTGACAGTGGAGATCCATGGCGTGAGCCGGGACGTGGCCAGCCGCCAGACTGCTGTCATCACCAAC |
| | | AATGGTTTCAACCCATGGTGGGACACGGAGTTTGCGTTTGAGGTAGTTGTGCCTGACCTTGCCCTCATCCGC |
| | | TTCTTGGTGGAAGATTATGATGCCTCCTCCAAGAATGACTTCATTGGCCAGAGTACCATCCCCTTGAACAGC |
| | | CTCAAGCAAGGATACCGCCATGTCCACCTC |
| PLCD3 | 47 | ACTCTCAGCATCCAGGTGCTGACTGCACAGCAGCTGCCCAAGCTGAATGCCGAGAAGCCACACTCCATTGTG |
| | | GACCCCCTGGTGCGCATTGAGATCCATGGGGTGCCCGCAGACTGTGCCCGGCAGGAGACTGACTACGTGCTC |
| | | AACAATGGCTTCAACCCCCGCTGGGGGCAGACCCTGCAGTTCCAGCTGCGGGCTCCGGAGCTGGCACTGGTC |
| | | CGGTTTGTGGTGGAAGATTATGACGCCACCTCCCCCAATGACTTTGTGGGCCAGTTTACACTGCCTCTTAGC |
| | | AGCCTAAAGCAAGGGTACCGCCACATACACCTG |
| PLCZ1 | 48 | ACACTTACAATAAGGCTCATCAGTGGTATCCAGTTGCCTCTTACTCATTCATCATCTAACAAAGGTGATTCA |
| | | TTAGTAATTATAGAAGTTTTTGGTGTTCCAAATGATCAAATGAAGCAGCAGACTCGTGTAATTAAAAAAAAT |
| | | GCTTTTAGTCCAAGATGGAATGAAACATTCACATTTATTATTCATGTCCCAGAATTGGCATTGATACGTTTT |
| | | GTTGTTGAAGGTCAAGGTTTAATAGCAGGAAATGAATTTCTTGGGCAATATACTTTGCCACTTCTATGCATG |
| | | AACAAAGGTTATCGTCGTATTCCTCTG |
| PTEN | 49 | TATAGACCAGTGGCACTGTTGTTTCACAAGATGATGTTTGAAACTATTCCAATGTTCAGTGGCGGAACTTGC |
| | | AATCCTCAGTTTGTGGTCTGCCAGCTAAAGGTGAAGATATATTCCTCCAATTCAGGACCCACACGACGGGAA |
| | | GACAAGTTCATGTACTTTGAGTTCCCTCAGCCGTTACCTGTGTGTGGTGATATCAAAGTAGAGTTCTTCCAC |
| | | AAACAGAACAAGATGCTAAAAAAGGACAAAATGTTTCACTTTTGGGTAAATACATTCTTCATACCAGGACCA |
| | | GAGGAAACCTCAGAAAAAGTAGAAAATGGAAGTCTATGTGATCAAGAAATCGATAGCATTTGCAGTATAGAG |
| | | CGTGCAGATAATGACAAGGAATATCTAGTACTTACTTTAACAAAAAATGATCTTGACAAAGCAAATAAAGAC |
| | | AAAGCCAACCGATACTTTTCTCCAAATTTTAAGGTGAAGCTGTACTTCACAAAA |
| RFIP1 | 50 | CACGTGCAGGTGACGGTGCTGCAGGCGCGGGGCCTGCGGGCCAAGGGCCCCGGGGGCACGAGCGACGCGTAC |
| | | GCGGTGATCCAGGTGGGCAAGGAGAAGTACGCCACCTCCGTGTCGGAGCGCAGCCTGGGCGCGCCCGTGTGG |
| | | CGCGAGGAGGCCACCTTCGAGCTGCCATCGCTGCTGTCCTCCGGACCCGCGGCCGCCGCCACCCTGCAGCTC |
| | | ACCGTGCTGCACCGCGCGCTGCTCGGCCTCGACAAGTTCCTGGGCCGCGCCGAGGTGGACCTGCGGGATCTG |
| | | CACCGCGACCAGGGCCGCAGGAAGACG |
| RFIP5 | 51 | CACGTCCAGGTGACGGTGCTGCGGGCCCGCGGGCTGCGGGGCAAGAGCTCGGGAGCGGGCAGCACCAGCGAC |
| | | GCGTACACGGTGATCCAGGTGGGCCGCGAGAAGTACAGTACGTCGGTGGTGGAGAAGACGCACGGCTGCCCC |
| | | GAGTGGCGTGAGGAGTGCTCCTTCGAGCTGCCGCCGGGGGCCCTGGATGGCCTGCTGCGGGCGCAGGAGGCC |
| | | GACGCGGGCCCGGCGCCCTGGGCCGCGAGCTCCGCCGCCGCCTGCGAGCTGGTGCTCACCACCATGCACCGC |
| | | TCGCTCATCGGCGTCGACAAGTTCCTGGGCCAGGCCACGGTGGCGCTGGACGAGGTCTTCGGCGCAGGCCGC |
| | | GCCCAGCACACG |
| RFIP2 | 52 | CACGTGCAGGTCACAGTGCTCCAAGCCAAAGATCTGAAGCCAAAAGGCAAAAGTGGTACCAATGACACATAC |
| | | ACTATAATTCAGCTGGGCAAGGAAAAGTACTCCACCTCTGTAGCTGAGAAAACCCTTGAGCCAGTTTGGAAG |
| | | GAGGAGGCCTCTTTCGAGCTACCTGGATTGCTAATTCAGGGAAGTCCAGAGAAATACATTCTTTTCCTTATA |
| | | GTTATGCACAGGTCCCTGGTGGGTCTGGATAAATTTTTAGGGCAGGTGGCAATCAATCTCAATGACATCTTT |
| | | GAGGACAAACAAAGAAGGAAAACA |
| SYT1 (C2 | 53 | CAGCTGCTGGTAGGGATCATTCAGGCTGCCGAACTGCCCGCCTTGGACATGGGGGGCACATCTGATCCTTAC |
| domain 1) | | GTGAAAGTGTTTCTGCTACCTGATAAGAAGAAGAAATTTGAGACAAAAGTCCACCGAAAAACCCTTAATCCT |
| | | GTCTTCAATGAGCAATTTACTTTCAAGGTACCATACTCGGAATTGGGTGGCAAAACCCTAGTGATGGCTGTA |
| | | TATGATTTTGATCGTTTCTCTAAGCATGACATCATTGGAGAATTTAAAGTCCCTATGAACACAGTGGATTTT |
| | | GGCCATGTAACTGAGGAATGG |
| SYT1 (C2 | 54 | AAGCTGACTGTTGTCATTCTGGAGGCAAAGAACCTGAAGAAGATGGATGTGGGTGGCTTATCCGATCCTTAT |
| domain 2) | | GTGAAGATTCATCTGATGCAGAATGGTAAGAGGCTGAAGAAGAAAAAGACAACAATTAAAAAGAACACACTT |
| | | AACCCCTACTACAATGAGTCATTCAGCTTTGAAGTACCTTTTGAACAAATCCAGAAAGTGCAGGTGGTGGTA |
| | | ACTGTTTTGGACTATGACAAGATTGGCAAGAACGATGCCATCGGCAAAGTCTTTGTGGGCTACAACAGCACC |
| | | GGCGCGGAGCTGCGACACTGGTCAGACATGCTGGCCAACCCCAGGCGACCTATTGCC |
| SYT2 | 55 | CAGCTTACTGTGGGCGTTCTGCAGGCTGCTGAACTGCCTGCCCTGGACATGGGAGGCACCTCAGACCCTTAT |
| | | GTCAAGGTCTTCCTCCTTCCTGACAAGAAGAAGAAATATGAGACCAAAGTCCATCGGAAGACACTGAACCCT |
| | | GCCTTCAATGAAACCTTCACCTTCAAGGTGCCATACCAGGAGCTTGGGGGCAAAACTCTGGTGATGGCCATC |
| | | TATGACTTTGACCGCTTCTCCAAACATGACATCATTGGAGAGGTAAAGGTGCCTATGAACACAGTGGACCTC |
| | | GGCCAGCCCATTGAGGAGTGG |
| SYT3 | 56 | CAGCTGGTGGTGAGGATCCTGCAGGCCCTGGACCTCCCTGCCAAGGACTCCAACGGCTTCTCAGACCCCTAC |
| | | GTCAAGATCTACCTGCTGCCTGACCGCAAGAAAAAGTTTCAGACCAAGGTGCACAGGAAGACCCTGAACCCC |
| | | GTCTTCAATGAGACGTTTCAATTCTCGGTGCCCCTGGCCGAGCTGGCCCAACGCAAACTGCACTTCAGCGTC |
| | | TATGACTTTGACCGCTTCTCGCGGCACGACCTCATCGGCCAGGTGGTGCTGGACAACCTCCTGGAGCTGGCC |
| | | GAGCAGCCCCCTGACCGCCCGCTC |
| SYT4 | 57 | GCATTTGTGGTCAATATCAAGGAAGCCCGTGGCTTGCCAGCCATGGATGAGCAGTCGATGACCTCTGACCCA |
| | | TATATCAAAATGACGATCCTCCCAGAGAAGAAGCATAAAGTGAAAACTAGAGTGCTGAGAAAAACCTTGGAT |
| | | CCAGCTTTTGATGAGACCTTTACATTCTATGGGATACCCTACACCCAAATCCAAGAATTGGCCTTGCACTTC |
| | | ACAATTTTGAGTTTTGACAGGTTTTCAAGAGATGATATCATTGGGGAAGTTCTAATTCCTCTCTCGGGAATT |
| | | GAATTATCTGAAGGAAAAATGTTAATG |
| SYT5 | 58 | CAGCTGCTGGTGGGCATTCTGCAAGCAATGGGATTGGCAGCCTTGGATCTTGGTGGCTCCTCGGACCCCTAT |
| | | GTGCGGGTCTACCTGCTGCCGGACAAACGGAGGCGGTACGAGACCAAGGTGCATCGGCAGACGCTGAACCCT |
| | | CACTTTGGGGAGACCTTCGCCTTCAAGGTCCCCTACGTGGAGCTGGGGGGCAGGGTGCTGGTCATGGCGGTG |
| | | TACGACTTCGACCGCTTCTCTCGCAATGACGCCATCGGGGAGGTGCGGGTCCCTATGAGCTCCGTGGACCTG |
| | | GGGCGGCCAGTGCAGGCCTGG |
| SYT6 | 59 | ACCCTGATTGTGCGTATCCTGAAGGCTTTTGACCTCCCTGCCAAGGACTTTTGTGGAAGCTCTGACCCTTAT |
| | | GTCAAGATCTACCTCCTGCCTGACCGCAAATGCAAGCTGCAGACCCGGGTGCACCGCAAGACCCTGAACCCC |
| | | ACCTTTGATGAGAACTTCCACTTCCCTGTGCCCTATGAGGAGCTGGCTGACCGCAAGCTGCATCTCAGTGTC |
| | | TTCGACTTTGACCGCTTCTCCCGCCATGACATGATTGGCGAGGTCATCCTGGACAACCTCTTTGAGGCCTCT |
| | | GACCTGTCTCGGGAAACCTCCATCTGG |
| SYT7 | 60 | ACGCTCACCGTGAAGATCATGAAGGCCCAGGAGCTGCCGGCCAAGGACTTCAGCGGCACCAGCGACCCCTTC |
| | | GTCAAGATCTACCTGCTGCCCGACAAGAAGCACAAGCTGGAGACCAAGGTGAAGCGGAAGAACCTGAACCCC |
| | | CACTGGAACGAGACCTTCCTCTTTGAAGGTTTTCCCTATGAGAAGGTGGTGCAGAGGATCCTCTACCTCCAA |
| | | GTCCTGGACTATGACCGCTTCAGCCGCAACGACCCCATTGGGGAGGTGTCCATCCCCCTTAACAAGGTGGAC |
| | | CTGACCCAGATGCAGACCTTCTGG |
| SYT9 | 61 | CAGCTCATAGTGAAGATTCACAAAGCTGTCAATTTGCCCGCCAAGGACTTTTCTGGGACTTCAGATCCTTAT |
| | | GTCAAGATCTATTTGCTTCCTGATCGGAAAACAAAACACCAGACTAAAGTTCACAGAAAGACCCTGAACCCT |
| | | GTGTTTGATGAAGTGTTTTTATTTCCGGTTCCCTACAATGACCTTGAAGCACGGAAGCTTCACTTCTCTGTG |
| | | TACGACTTTGACAGGTTCTCTCGTCATGACTTAATCGGCCAAGTGGTGGTGGATCACTTCCTAGACTTGGCT |
| | | GATTTCCCCAGGGAGTGCATCCTT |
| SYT10 | 62 | CTTCTAGTTGTTAAAATTATCAAAGCTTTAGATCTCCCTGCTAAAGACTTCACAGGAACTTCTGACCCTTAT |
| | | GTGAAGATGTATCTTCTTCCAGATAGGAAAAAGAAATTTCAGACCCGCGTGCACAGAAAGACTTTAAATCCT |
| | | CTATTTGATGAAACTTTTCAATTTCCTGTAGCATATGATCAACTAAGCAACCGAAAACTACATTTCAGTGTG |
| | | TATGATTTTGACAGATTTTCTAGACATGACATGATTGGGGAAGTGATTCTTGATAATTTGTTTGAAGTCTCT |
| | | GATCTCTCCAGGGAAGCCACAGTA |
| ESYT1 | 63 | AAGCTGGTCAGCATTGTTCATGGTTGCCGGTCCCTTCGACAGAATGGACGTGATCCTCCTGATCCCTATGTG |
| | | TCACTGTTGCTACTGCCAGACAAGAACCGAGGCACCAAGAGGAGGACCTCACAGAAGAAGAGGACCCTGAGT |
| | | CCTGAATTTAATGAACGGTTTGAGTGGGAACTCCCCCTGGATGAGGCCCAGAGACGAAAGCTGGATGTCTCT |
| | | GTCAAGTCTAATTCCTCCTTCATGTCAAGAGAGCGTGAGCTGCTGGGGAAGGTGCAGCTGGACCTAGCTGAG |
| | | ACAGACCTTTCCCAGGGTGTAGCCCGGTGG |
| ESYT2 | 64 | CTGGGGCAGATCCAGCTGACCATCCGGCACAGCTCGCAGAGAAACAAGCTTATCGTGGTCGTGCATGCCTGC |
| | | AGAAACCTCATTGCCTTCTCTGAAGACGGCTCTGACCCCTATGTCCGCATGTATTTATTACCAGACAAGAGG |
| | | CGGTCAGGAAGGAGGAAAACACACGTGTCAAAGAAAACATTAAATCCAGTGTTTGATCAAAGCTTTGATTTC |
| | | AGTGTTTCGTTACCAGAAGTGCAGAGGAGAACGCTCGACGTTGCCGTGAAGAACAGTGGCGGCTTCCTGTCC |
| | | AAAGACAAAGGGCTCCTTGGCAAAGTATTGGTTGCTCTGGCATCTGAAGAACTTGCCAAAGGCTGGACCCAG |
| | | TGGTATGACCTCACGGAAGATGGGACG |
| ESYT3 | 65 | TGCCTCAGCGTGCTAATCAATGGCTGCAGAAACCTAACACCATGTACCAGCAGTGGAGCTGATCCCTACGTC |
| | | CGTGTCTACTTGTTGCCAGAAAGGAAGTGGGCATGTCGTAAGAAGACTTCAGTGAAGCGGAAGACCTTGGAA |
| | | CCCCTGTTTGATGAGACATTTGAATTTTTTGTTCCCATGGAAGAAGTAAAGAAGAGGTCACTAGATGTTGCA |
| | | GTGAAAAATAGTAGGCCACTTGGCTCACACAGAAGAAAGGAGTTAGGAAAAGTACTGATTGACTTATCAAAA |
| | | GAAGATCTGATTAAGGGCTTTTCACAATGGTATGAG |
| DYSF | 66 | ATGCTGTGCTGCCTGCTGGTGAGGGCCAGCAACCTCCCCAGTGCGAAGAAGGACCGGCGCAGCGACCCTGTC |
| | | GCAAGCCTGACTTTCCGAGGGGTGAAGAAGAGAACCAAAGTCATCAAGAACAGCGTGAACCCTGTATGGAAT |
| | | GAGGGATTTGAATGGGACCTCAAGGGCATCCCCCTGGACCAGGGCTCTGAGCTTCATGTGGTGGTCAAAGAC |
| | | CATGAGACGATGGGGAGGAACAGGTTCCTGGGGGAAGCCAAGGTCCCACTCCGAGAGGTCCTCGCCACCCCT |
| | | AGTCTGTCCGCCAGC |
| MYOF | 67 | ATGCTGCGAGTGATTGTGGAATCTGCCAGCAATATCCCTAAAACGAAATTTGGCAAGCCGGATCCTATTGTT |
| | | TCTGTCATTTTTAAGGATGAGAAAAAGAAAACAAAGAAAGTTGATAATGAATTGAACCCTGTCTGGAATGAG |
| | | ATTTTGGAGTTTGACTTGAGGGGTATACCACTGGACTTTTCATCTTCCCTTGGGATTATTGTGAAAGATTTT |
| | | GAGACAATTGGACAAAATAAATTAATTGGCACGGCGACTGTAGCCCTGAAGGACCTGACTGGTGACCAGAGC |
| | | AGATCCCTGCCG |
| FR1L5 | 68 | CAGGTTCGAGTGAAGGTGTTTGAAGCCCGACAGCTCATGGGCAACAACATCAAACCAGTGGTGAAGGTGTCC |
| | | ATCGCAGGCCAGCAGCACCAGACACGCATCAAGATGGGAAACAACCCTTTCTTTAATGAGATCTTCTTCCAG |
| | | AATTTTCATGAGGTTCCTGCAAAGTTCTTTGATGAGACCATCTTAATCCAGACAGATATTGGGTTTATCTAC |
| | | CATTCTCCAGGTCACACACTCCTAAGGAAATGGCTAGGCCTCTGCCAGCCAAATAACCCTGGCAGTGGT |

TABLE 4
C2 Domain-Containing Constructs

| | SEQ | | |
|---|---|---|---|
| Insertion | ID NO: | C2 domain: | BoNT/A with C2 Domain |
| N-terminal | 69 | RP3A (C2 | ATGTCCCTGCAGTGCACCATCATTAAGGCCAAGGGCCTGAAGCCCATGGATTCAAACGGCTTGGC |
| | | domain 1) | TGATCCCTACGTTAAGCTGCACCTCCTGCCGGGAGCCAGCAAGTCCAACAAGCTTCGTACAAAAA |
| | | | CTCTGCGGAATACCCGGAACCCCATCTGGAATGAGACCCTCGTGTATCACGGCATCACCGATGAG |
| | | | GACATGCAAAGGAAGACCCTCAGGATCTCCGTCTGTGATGAGGACAAATTTGGCCACAATGAATT |
| | | | TATTGGTGAGACCAGATTCTCCCTCAAGAAACTGAAGCCCAACCAGAGGAAGAATTTCAACCCAT |
| | | | TTGTTAATAAACAATTTAATTATAAAGATCCTGTAAATGGTGTTGATATTGCTTATATAAAAATT |
| | | | CCAAATGCAGGACAAATGCAACCAGTAAAAGCTTTTAAAATTCATAATAAAATATGGGTTATTCC |
| | | | AGAAAGAGATACATTTACAAATCCTGAAGAAGGAGATTTAAATCCACCACCAGAAGCAAAACAAG |
| | | | TTCCAGTTTCATATTATGATTCAACATATTTAAGTACAGATAATGAAAAAGATAATTATTTAAAG |
| | | | GGAGTTACAAAATTATTTGAGAGAATTTATTCAACTGATCTTGGAAGAATGTTGTTAACATCAAT |
| | | | AGTAAGGGGAATACCATTTTGGGGTGGAAGTACAATAGATACAGAATTAAAAGTTATTGATACTA |
| | | | ATTGTATTAATGTGATACAACCAGATGGTAGTTATAGATCAGAAGAACTTAATCTAGTAATAATA |
| | | | GGACCCTCAGCTGATATTATACAGTTTGAATGTAAAAGCTTTGGACATGAAGTTTTGAATCTTAC |
| | | | GCGAAATGGTTATGGCTCTACTCAATACATTAGATTTAGCCCAGATTTTACATTTGGTTTTGAGG |
| | | | AGTCACTTGAAGTTGATACAAATCCTCTTTTAGGTGCAGGCAAATTTGCTACAGATCCAGCAGTA |
| | | | ACATTAGCACATGAACTTATACATGCTGGACATAGATTATATGGAATAGCAATTAATCCAAATAG |
| | | | GGTTTTTAAAGTAAATACTAATGCCTATTATGAAATGAGTGGGTTAGAAGTAAGCTTTGAGGAAC |
| | | | TTAGAACATTTGGGGGACATGATGCAAAGTTTATAGATAGTTTACAGGAAAACGAATTTCGTCTA |
| | | | TATTATTATAATAAGTTTAAAGATATAGCAAGTACACTTAATAAAGCTAAATCAATAGTAGGTAC |
| | | | TACTGCTTCATTACAGTATATGAAAAATGTTTTTAAAGAGAAATATCTCCTATCTGAAGATACAT |
| | | | CTGGAAAATTTTCGGTAGATAAATTAAAATTTGATAAGTTATACAAAATGTTAACAGAGATTTAC |
| | | | ACAGAGGATAATTTTGTTAAGTTTTTTAAAGTACTTAACAGAAAAACATATTTGAATTTTGATAA |
| | | | AGCCGTATTTAAGATAAATATAGTACCTAAGGTAAATTACACAATATATGATGGATTTAATTTAA |
| | | | GAAATACAAATTTAGCAGCAAACTTTAATGGTCAAAATACAGAAATTAATAATATGAATTTTACT |
| | | | AAACTAAAAAATTTTACTGGATTGTTTGAATTTTATAAGTTGCTATGTGTGCGCGGCATCATTAC |
| | | | CAGCAAGGCAGGTGCGGGCAAGTCCTTGGTTCCGCGTGGCAGCGCCGGCGCCGGCGCGCTCAATG |
| | | | ATCTGTGTATCAAAGTTAATAATTGGGACTTGTTTTTTAGTCCTTCAGAAGATAATTTTACTAAT |
| | | | GATCTAAATAAAGGAGAAGAAATTACATCTGATACTAATATAGAAGCAGCAGAAGAAAATATTAG |
| | | | TTTAGATTTAATACAACAATATTATTTAACCTTTAATTTTGATAATGAACCTGAAAATATTTCAA |
| | | | TAGAAAATCTTTCAAGTGACATTATAGGCCAATTAGAACTTATGCCTAATATAGAAAGATTTCCT |
| | | | AATGGAAAAAAGTATGAGTTAGATAAATATACTATGTTCCATTATCTTCGTGCTCAAGAATTTGA |
| | | | ACATGGTAAATCTAGGATTGCTTTAACAAATTCTGTTAACGAAGCATTATTAAATCCTAGTCGTG |
| | | | TTTATACATTTTTTTCTTCAGACTATGTAAAGAAAGTTAATAAAGCTACGGAGGCAGCTATGTTT |
| | | | TTAGGCTGGGTAGAACAATTAGTATATGATTTTACCGATGAAACTAGCGAAGTAAGTACTACGGA |
| | | | TAAAATTGCGGATATAACTATAATTATTCCATATATAGGACCTGCTTTAAATATAGGTAATATGT |
| | | | TATATAAAGATGATTTTGTAGGTGCTTTAATATTTTCAGGAGCTGTTATTCTGTTAGAATTTATA |
| | | | CCAGAGATTGCAATACCTGTATTAGGTACTTTTGCACTTGTATCATATATTGCGAATAAGGTTCT |
| | | | AACCGTTCAAACAATAGATAATGCTTTAAGTAAAAGAAATGAAAAATGGGATGAGGTCTATAAAT |
| | | | ATATAGTAACAAATTGGTTAGCAAAGGTTAATACACAGATTGATCTAATAAGAAAAAAAATGAAA |
| | | | GAAGCTTTAGAAAATCAAGCAGAAGCAACAAAGGCTATAATAAACTATCAGTATAATCAATATAC |
| | | | TGAGGAAGAGAAAAATAATATTAATTTTAATATTGATGATTTAAGTTCGAAACTTAATGAGTCTA |
| | | | TAAATAAAGCTATGATTAATATAAATAAATTTTTGAATCAATGCTCTGTTTCATATTTAATGAAT |
| | | | TCTATGATCCCTTATGGTGTTAAACGGTTAGAAGATTTTGATGCTAGTCTTAAAGATGCATTATT |
| | | | AAAGTATATATATGATAATAGAGGAACTTTAATTGGTCAAGTAGATAGATTAAAAGATAAAGTTA |
| | | | ATAATACACTTAGTACAGATATACCTTTTCAGCTTTCCAAATACGTAGATAATCAAAGATTATTA |
| | | | TCTACATTTACTGAATATATTAAGAATATTATTAATACTTCTATATTGAATTTAAGATATGAAAG |
| | | | TAATCATTTAATAGACTTATCTAGGTATGCATCAAAAATAAATATTGGTAGTAAAGTAAATTTTG |
| | | | ATCCAATAGATAAAAATCAAATTCAATTATTTAATTTAGAAAGTAGTAAAATTGAGGTAATTTTA |
| | | | AAAAATGCTATTGTATATAATAGTATGTATGAAAATTTTAGTACTAGCTTTTGGATAAGAATTCC |
| | | | TAAGTATTTTAACAGTATAAGTCTAAATAATGAATATACAATAATAAATTGTATGGAAAATAATT |
| | | | CAGGATGGAAAGTATCACTTAATTATGGTGAAATAATCTGGACTTTACAGGATACTCAGGAAATA |
| | | | AAACAAAGAGTAGTTTTTAAATACAGTCAAATGATTAATATATCAGATTATATAAACAGATGGAT |
| | | | TTTTGTAACTATCACTAATAATAGATTAAATAACTCTAAAATTTATATAAATGGAAGATTAATAG |
| | | | ATCAAAAACCAATTTCAAATTTAGGTAATATTCATGCTAGTAATAATATAATGTTTAAATTAGAT |
| | | | GGTTGTAGAGATACACATAGATATATTTGGATAAAATATTTTAATCTTTTTGATAAGGAATTAAA |
| | | | TGAAAAAGAAATCAAAGATTTATATGATAATCAATCAAATTCAGGTATTTTAAAAGACTTTTGGG |
| | | | GTGATTATTTACAATATGATAAACCATACTATATGTTAAATTTATATGATCCAAATAAATATGTC |
| | | | GATGTAAATAATGTAGGTATTAGAGGTTATATGTATCTTAAAGGGCCTAGAGGTAGCGTAATGAC |
| | | | TACAAACATTTATTTAAATTCAAGTTTGTATAGGGGGACAAAATTTATTATAAAAAAATATGCTT |
| | | | CTGGAAATAAAGATAATATTGTTAGAAATAATGATCGTGTATATATTAATGTAGTAGTTAAAAAT |
| | | | AAAGAATATAGGTTAGCTACTAATGCATCACAGGCAGGCGTAGAAAAAATACTAAGTGCATTAGA |
| | | | AATACCTGATGTAGGAAATCTAAGTCAAGTAGTAGTAATGAAGTCAAAAAATGATCAAGGAATAA |
| | | | CAAATAAATGCAAAATGAATTTACAAGATAATAATGGGAATGATATAGGCTTTATAGGATTTCAT |
| | | | CAGTTTAATAATATAGCTAAACTAGTAGCAAGTAATTGGTATAATAGACAAATAGAAAGATCTAG |
| | | | TAGGACTTTGGGTTGCTCATGGGAATTTATTCCTGTAGATGATGGATGGGGAGAAAGGCCACTGT |
| | | | AA |
| N-terminal | 70 | ESYT2 | ATGCTGGGGCAGATCCAGCTGACCATCCGGCACAGCTCGCAGAGAAACAAGCTTATCGTGGTCGT |
| | | | GCATGCCTGCAGAAACCTCATTGCCTTCTCTGAAGACGGCTCTGACCCCTATGTCCGCATGTATT |
| | | | TATTACCAGACAAGAGGCGGTCAGGAAGGAGGAAAACACACGTGTCAAAGAAAACATTAAATCCA |
| | | | GTGTTTGATCAAAGCTTTGATTTCAGTGTTTCGTTACCAGAAGTGCAGAGGAGAACGCTCGACGT |
| | | | TGCCGTGAAGAACAGTGGCGGCTTCCTGTCCAAAGACAAAGGGCTCCTTGGCAAAGTATTGGTTG |
| | | | CTCTGGCATCTGAAGAACTTGCCAAAGGCTGGACCCAGTGGTATGACCTCACGGAAGATGGGACG |
| | | | CCATTTGTTAATAAACAATTTAATTATAAAGATCCTGTAAATGGTGTTGATATTGCTTATATAAA |
| | | | AATTCCAAATGCAGGACAAATGCAACCAGTAAAAGCTTTTAAAATTCATAATAAAATATGGGTTA |
| | | | TTCCAGAAAGAGATACATTTACAAATCCTGAAGAAGGAGATTTAAATCCACCACCAGAAGCAAAA |
| | | | CAAGTTCCAGTTTCATATTATGATTCAACATATTTAAGTACAGATAATGAAAAAGATAATTATTT |
| | | | AAAGGGAGTTACAAAATTATTTGAGAGAATTTATTCAACTGATCTTGGAAGAATGTTGTTAACAT |
| | | | CAATAGTAAGGGGAATACCATTTTGGGGTGGAAGTACAATAGATACAGAATTAAAAGTTATTGAT |
| | | | ACTAATTGTATTAATGTGATACAACCAGATGGTAGTTATAGATCAGAAGAACTTAATCTAGTAAT |
| | | | AATAGGACCCTCAGCTGATATTATACAGTTTGAATGTAAAAGCTTTGGACATGAAGTTTTGAATC |
| | | | TTACGCGAAATGGTTATGGCTCTACTCAATACATTAGATTTAGCCCAGATTTTACATTTGGTTTT |
| | | | GAGGAGTCACTTGAAGTTGATACAAATCCTCTTTTAGGTGCAGGCAAATTTGCTACAGATCCAGC |
| | | | AGTAACATTAGCACATGAACTTATACATGCTGGACATAGATTATATGGAATAGCAATTAATCCAA |
| | | | ATAGGGTTTTTAAAGTAAATACTAATGCCTATTATGAAATGAGTGGGTTAGAAGTAAGCTTTGAG |
| | | | GAACTTAGAACATTTGGGGGACATGATGCAAAGTTTATAGATAGTTTACAGGAAAACGAATTTCG |
| | | | TCTATATTATTATAATAAGTTTAAAGATATAGCAAGTACACTTAATAAAGCTAAATCAATAGTAG |
| | | | GTACTACTGCTTCATTACAGTATATGAAAAATGTTTTTAAAGAGAAATATCTCCTATCTGAAGAT |
| | | | ACATCTGGAAAATTTTCGGTAGATAAATTAAAATTTGATAAGTTATACAAAATGTTAACAGAGAT |
| | | | TTACACAGAGGATAATTTTGTTAAGTTTTTTAAAGTACTTAACAGAAAAACATATTTGAATTTTG |
| | | | ATAAAGCCGTATTTAAGATAAATATAGTACCTAAGGTAAATTACACAATATATGATGGATTTAAT |
| | | | TTAAGAAATACAAATTTAGCAGCAAACTTTAATGGTCAAAATACAGAAATTAATAATATGAATTT |
| | | | TACTAAACTAAAAAATTTTACTGGATTGTTTGAATTTTATAAGTTGCTATGTGTGCGCGGCATCA |
| | | | TTACCAGCAAGGCAGGTGCGGGCAAGTCCTTGGTTCCGCGTGGCAGCGCCGGCGCCGGCGCGCTC |
| | | | AATGATCTGTGTATCAAAGTTAATAATTGGGACTTGTTTTTTAGTCCTTCAGAAGATAATTTTAC |
| | | | TAATGATCTAAATAAAGGAGAAGAAATTACATCTGATACTAATATAGAAGCAGCAGAAGAAAATA |
| | | | TTAGTTTAGATTTAATACAACAATATTATTTAACCTTTAATTTTGATAATGAACCTGAAAATATT |
| | | | TCAATAGAAAATCTTTCAAGTGACATTATAGGCCAATTAGAACTTATGCCTAATATAGAAAGATT |
| | | | TCCTAATGGAAAAAAGTATGAGTTAGATAAATATACTATGTTCCATTATCTTCGTGCTCAAGAAT |
| | | | TTGAACATGGTAAATCTAGGATTGCTTTAACAAATTCTGTTAACGAAGCATTATTAAATCCTAGT |
| | | | CGTGTTTATACATTTTTTTCTTCAGACTATGTAAAGAAAGTTAATAAAGCTACGGAGGCAGCTAT |
| | | | GTTTTTAGGCTGGGTAGAACAATTAGTATATGATTTTACCGATGAAACTAGCGAAGTAAGTACTA |
| | | | CGGATAAAATTGCGGATATAACTATAATTATTCCATATATAGGACCTGCTTTAAATATAGGTAAT |
| | | | ATGTTATATAAAGATGATTTTGTAGGTGCTTTAATATTTTCAGGAGCTGTTATTCTGTTAGAATT |
| | | | TATACCAGAGATTGCAATACCTGTATTAGGTACTTTTGCACTTGTATCATATATTGCGAATAAGG |
| | | | TTCTAACCGTTCAAACAATAGATAATGCTTTAAGTAAAAGAAATGAAAAATGGGATGAGGTCTAT |
| | | | AAATATATAGTAACAAATTGGTTAGCAAAGGTTAATACACAGATTGATCTAATAAGAAAAAAAAT |
| | | | GAAAGAAGCTTTAGAAAATCAAGCAGAAGCAACAAAGGCTATAATAAACTATCAGTATAATCAAT |
| | | | ATACTGAGGAAGAGAAAAATAATATTAATTTTAATATTGATGATTTAAGTTCGAAACTTAATGAG |
| | | | TCTATAAATAAAGCTATGATTAATATAAATAAATTTTTGAATCAATGCTCTGTTTCATATTTAAT |
| | | | GAATTCTATGATCCCTTATGGTGTTAAACGGTTAGAAGATTTTGATGCTAGTCTTAAAGATGCAT |
| | | | TATTAAAGTATATATATGATAATAGAGGAACTTTAATTGGTCAAGTAGATAGATTAAAAGATAAA |
| | | | GTTAATAATACACTTAGTACAGATATACCTTTTCAGCTTTCCAAATACGTAGATAATCAAAGATT |
| | | | ATTATCTACATTTACTGAATATATTAAGAATATTATTAATACTTCTATATTGAATTTAAGATATG |
| | | | AAAGTAATCATTTAATAGACTTATCTAGGTATGCATCAAAAATAAATATTGGTAGTAAAGTAAAT |
| | | | TTTGATCCAATAGATAAAAATCAAATTCAATTATTTAATTTAGAAAGTAGTAAAATTGAGGTAAT |
| | | | TTTAAAAAATGCTATTGTATATAATAGTATGTATGAAAATTTTAGTACTAGCTTTTGGATAAGAA |
| | | | TTCCTAAGTATTTTAACAGTATAAGTCTAAATAATGAATATACAATAATAAATTGTATGGAAAAT |
| | | | AATTCAGGATGGAAAGTATCACTTAATTATGGTGAAATAATCTGGACTTTACAGGATACTCAGGA |
| | | | AATAAAACAAAGAGTAGTTTTTAAATACAGTCAAATGATTAATATATCAGATTATATAAACAGAT |
| | | | GGATTTTTGTAACTATCACTAATAATAGATTAAATAACTCTAAAATTTATATAAATGGAAGATTA |
| | | | ATAGATCAAAAACCAATTTCAAATTTAGGTAATATTCATGCTAGTAATAATATAATGTTTAAATT |
| | | | AGATGGTTGTAGAGATACACATAGATATATTTGGATAAAATATTTTAATCTTTTTGATAAGGAAT |
| | | | TAAATGAAAAAGAAATCAAAGATTTATATGATAATCAATCAAATTCAGGTATTTTAAAAGACTTT |
| | | | TGGGGTGATTATTTACAATATGATAAACCATACTATATGTTAAATTTATATGATCCAAATAAATA |
| | | | TGTCGATGTAAATAATGTAGGTATTAGAGGTTATATGTATCTTAAAGGGCCTAGAGGTAGCGTAA |
| | | | TGACTACAAACATTTATTTAAATTCAAGTTTGTATAGGGGGACAAAATTTATTATAAAAAAATAT |
| | | | GCTTCTGGAAATAAAGATAATATTGTTAGAAATAATGATCGTGTATATATTAATGTAGTAGTTAA |
| | | | AAATAAAGAATATAGGTTAGCTACTAATGCATCACAGGCAGGCGTAGAAAAAATACTAAGTGCAT |
| | | | TAGAAATACCTGATGTAGGAAATCTAAGTCAAGTAGTAGTAATGAAGTCAAAAAATGATCAAGGA |
| | | | ATAACAAATAAATGCAAAATGAATTTACAAGATAATAATGGGAATGATATAGGCTTTATAGGATT |
| | | | TCATCAGTTTAATAATATAGCTAAACTAGTAGCAAGTAATTGGTATAATAGACAAATAGAAAGAT |
| | | | CTAGTAGGACTTTGGGTTGCTCATGGGAATTTATTCCTGTAGATGATGGATGGGGAGAAAGGCCA |
| | | | CTGTAA |
| Linker | 71 | RP3A (C2 | ATGCCATTTGTTAATAAACAATTTAATTATAAAGATCCTGTAAATGGTGTTGATATTGCTTATAT |
| region | | domain 1) | AAAAATTCCAAATGCAGGACAAATGCAACCAGTAAAAGCTTTTAAAATTCATAATAAAATATGGG |
| | | | TTATTCCAGAAAGAGATACATTTACAAATCCTGAAGAAGGAGATTTAAATCCACCACCAGAAGCA |
| | | | AAACAAGTTCCAGTTTCATATTATGATTCAACATATTTAAGTACAGATAATGAAAAAGATAATTA |
| | | | TTTAAAGGGAGTTACAAAATTATTTGAGAGAATTTATTCAACTGATCTTGGAAGAATGTTGTTAA |
| | | | CATCAATAGTAAGGGGAATACCATTTTGGGGTGGAAGTACAATAGATACAGAATTAAAAGTTATT |
| | | | GATACTAATTGTATTAATGTGATACAACCAGATGGTAGTTATAGATCAGAAGAACTTAATCTAGT |
| | | | AATAATAGGACCCTCAGCTGATATTATACAGTTTGAATGTAAAAGCTTTGGACATGAAGTTTTGA |
| | | | ATCTTACGCGAAATGGTTATGGCTCTACTCAATACATTAGATTTAGCCCAGATTTTACATTTGGT |
| | | | TTTGAGGAGTCACTTGAAGTTGATACAAATCCTCTTTTAGGTGCAGGCAAATTTGCTACAGATCC |
| | | | AGCAGTAACATTAGCACATGAACTTATACATGCTGGACATAGATTATATGGAATAGCAATTAATC |
| | | | CAAATAGGGTTTTTAAAGTAAATACTAATGCCTATTATGAAATGAGTGGGTTAGAAGTAAGCTTT |
| | | | GAGGAACTTAGAACATTTGGGGGACATGATGCAAAGTTTATAGATAGTTTACAGGAAAACGAATT |
| | | | TCGTCTATATTATTATAATAAGTTTAAAGATATAGCAAGTACACTTAATAAAGCTAAATCAATAG |
| | | | TAGGTACTACTGCTTCATTACAGTATATGAAAAATGTTTTTAAAGAGAAATATCTCCTATCTGAA |
| | | | GATACATCTGGAAAATTTTCGGTAGATAAATTAAAATTTGATAAGTTATACAAAATGTTAACAGA |
| | | | GATTTACACAGAGGATAATTTTGTTAAGTTTTTTAAAGTACTTAACAGAAAAACATATTTGAATT |
| | | | TTGATAAAGCCGTATTTAAGATAAATATAGTACCTAAGGTAAATTACACAATATATGATGGATTT |
| | | | AATTTAAGAAATACAAATTTAGCAGCAAACTTTAATGGTCAAAATACAGAAATTAATAATATGAA |
| | | | TTTTACTAAACTAAAAAATTTTACTGGATTGTTTGAATTTTATAAGTTGCTATGTGTGCGCGGCA |
| | | | TCATTACCTCCCTGCAGTGCACCATCATTAAGGCCAAGGGCCTGAAGCCCATGGATTCAAACGGC |
| | | | TTGGCTGATCCCTACGTTAAGCTGCACCTCCTGCCGGGAGCCAGCAAGTCCAACAAGCTTCGTAC |
| | | | AAAAACTCTGCGGAATACCCGGAACCCCATCTGGAATGAGACCCTCGTGTATCACGGCATCACCG |
| | | | ATGAGGACATGCAAAGGAAGACCCTCAGGATCTCCGTCTGTGATGAGGACAAATTTGGCCACAAT |
| | | | GAATTTATTGGTGAGACCAGATTCTCCCTCAAGAAACTGAAGCCCAACCAGAGGAAGAATTTCAA |
| | | | CAGCAAGGCAGGTGCGGGCAAGTCCTTGGTTCCGCGTGGCAGCGCCGGCGCCGGCGCGCTCAATG |
| | | | ATCTGTGTATCAAAGTTAATAATTGGGACTTGTTTTTTAGTCCTTCAGAAGATAATTTTACTAAT |
| | | | GATCTAAATAAAGGAGAAGAAATTACATCTGATACTAATATAGAAGCAGCAGAAGAAAATATTAG |
| | | | TTTAGATTTAATACAACAATATTATTTAACCTTTAATTTTGATAATGAACCTGAAAATATTTCAA |
| | | | TAGAAAATCTTTCAAGTGACATTATAGGCCAATTAGAACTTATGCCTAATATAGAAAGATTTCCT |
| | | | AATGGAAAAAAGTATGAGTTAGATAAATATACTATGTTCCATTATCTTCGTGCTCAAGAATTTGA |
| | | | ACATGGTAAATCTAGGATTGCTTTAACAAATTCTGTTAACGAAGCATTATTAAATCCTAGTCGTG |
| | | | TTTATACATTTTTTTCTTCAGACTATGTAAAGAAAGTTAATAAAGCTACGGAGGCAGCTATGTTT |
| | | | TTAGGCTGGGTAGAACAATTAGTATATGATTTTACCGATGAAACTAGCGAAGTAAGTACTACGGA |
| | | | TAAAATTGCGGATATAACTATAATTATTCCATATATAGGACCTGCTTTAAATATAGGTAATATGT |
| | | | TATATAAAGATGATTTTGTAGGTGCTTTAATATTTTCAGGAGCTGTTATTCTGTTAGAATTTATA |
| | | | CCAGAGATTGCAATACCTGTATTAGGTACTTTTGCACTTGTATCATATATTGCGAATAAGGTTCT |
| | | | AACCGTTCAAACAATAGATAATGCTTTAAGTAAAAGAAATGAAAAATGGGATGAGGTCTATAAAT |
| | | | ATATAGTAACAAATTGGTTAGCAAAGGTTAATACACAGATTGATCTAATAAGAAAAAAAATGAAA |
| | | | GAAGCTTTAGAAAATCAAGCAGAAGCAACAAAGGCTATAATAAACTATCAGTATAATCAATATAC |
| | | | TGAGGAAGAGAAAAATAATATTAATTTTAATATTGATGATTTAAGTTCGAAACTTAATGAGTCTA |
| | | | TAAATAAAGCTATGATTAATATAAATAAATTTTTGAATCAATGCTCTGTTTCATATTTAATGAAT |
| | | | TCTATGATCCCTTATGGTGTTAAACGGTTAGAAGATTTTGATGCTAGTCTTAAAGATGCATTATT |
| | | | AAAGTATATATATGATAATAGAGGAACTTTAATTGGTCAAGTAGATAGATTAAAAGATAAAGTTA |
| | | | ATAATACACTTAGTACAGATATACCTTTTCAGCTTTCCAAATACGTAGATAATCAAAGATTATTA |
| | | | TCTACATTTACTGAATATATTAAGAATATTATTAATACTTCTATATTGAATTTAAGATATGAAAG |
| | | | TAATCATTTAATAGACTTATCTAGGTATGCATCAAAAATAAATATTGGTAGTAAAGTAAATTTTG |
| | | | ATCCAATAGATAAAAATCAAATTCAATTATTTAATTTAGAAAGTAGTAAAATTGAGGTAATTTTA |
| | | | AAAAATGCTATTGTATATAATAGTATGTATGAAAATTTTAGTACTAGCTTTTGGATAAGAATTCC |
| | | | TAAGTATTTTAACAGTATAAGTCTAAATAATGAATATACAATAATAAATTGTATGGAAAATAATT |
| | | | CAGGATGGAAAGTATCACTTAATTATGGTGAAATAATCTGGACTTTACAGGATACTCAGGAAATA |
| | | | AAACAAAGAGTAGTTTTTAAATACAGTCAAATGATTAATATATCAGATTATATAAACAGATGGAT |
| | | | TTTTGTAACTATCACTAATAATAGATTAAATAACTCTAAAATTTATATAAATGGAAGATTAATAG |
| | | | ATCAAAAACCAATTTCAAATTTAGGTAATATTCATGCTAGTAATAATATAATGTTTAAATTAGAT |
| | | | GGTTGTAGAGATACACATAGATATATTTGGATAAAATATTTTAATCTTTTTGATAAGGAATTAAA |
| | | | TGAAAAAGAAATCAAAGATTTATATGATAATCAATCAAATTCAGGTATTTTAAAAGACTTTTGGG |
| | | | GTGATTATTTACAATATGATAAACCATACTATATGTTAAATTTATATGATCCAAATAAATATGTC |
| | | | GATGTAAATAATGTAGGTATTAGAGGTTATATGTATCTTAAAGGGCCTAGAGGTAGCGTAATGAC |
| | | | TACAAACATTTATTTAAATTCAAGTTTGTATAGGGGGACAAAATTTATTATAAAAAAATATGCTT |
| | | | CTGGAAATAAAGATAATATTGTTAGAAATAATGATCGTGTATATATTAATGTAGTAGTTAAAAAT |
| | | | AAAGAATATAGGTTAGCTACTAATGCATCACAGGCAGGCGTAGAAAAAATACTAAGTGCATTAGA |
| | | | AATACCTGATGTAGGAAATCTAAGTCAAGTAGTAGTAATGAAGTCAAAAAATGATCAAGGAATAA |
| | | | CAAATAAATGCAAAATGAATTTACAAGATAATAATGGGAATGATATAGGCTTTATAGGATTTCAT |
| | | | CAGTTTAATAATATAGCTAAACTAGTAGCAAGTAATTGGTATAATAGACAAATAGAAAGATCTAG |
| | | | TAGGACTTTGGGTTGCTCATGGGAATTTATTCCTGTAGATGATGGATGGGGAGAAAGGCCACTGT |
| | | | AA |
| Linker | 72 | ESYT2 | ATGCCATTTGTTAATAAACAATTTAATTATAAAGATCCTGTAAATGGTGTTGATATTGCTTATAT |
| region | | | AAAAATTCCAAATGCAGGACAAATGCAACCAGTAAAAGCTTTTAAAATTCATAATAAAATATGGG |
| | | | TTATTCCAGAAAGAGATACATTTACAAATCCTGAAGAAGGAGATTTAAATCCACCACCAGAAGCA |
| | | | AAACAAGTTCCAGTTTCATATTATGATTCAACATATTTAAGTACAGATAATGAAAAAGATAATTA |
| | | | TTTAAAGGGAGTTACAAAATTATTTGAGAGAATTTATTCAACTGATCTTGGAAGAATGTTGTTAA |
| | | | CATCAATAGTAAGGGGAATACCATTTTGGGGTGGAAGTACAATAGATACAGAATTAAAAGTTATT |
| | | | GATACTAATTGTATTAATGTGATACAACCAGATGGTAGTTATAGATCAGAAGAACTTAATCTAGT |
| | | | AATAATAGGACCCTCAGCTGATATTATACAGTTTGAATGTAAAAGCTTTGGACATGAAGTTTTGA |
| | | | ATCTTACGCGAAATGGTTATGGCTCTACTCAATACATTAGATTTAGCCCAGATTTTACATTTGGT |
| | | | TTTGAGGAGTCACTTGAAGTTGATACAAATCCTCTTTTAGGTGCAGGCAAATTTGCTACAGATCC |
| | | | AGCAGTAACATTAGCACATGAACTTATACATGCTGGACATAGATTATATGGAATAGCAATTAATC |
| | | | CAAATAGGGTTTTTAAAGTAAATACTAATGCCTATTATGAAATGAGTGGGTTAGAAGTAAGCTTT |
| | | | GAGGAACTTAGAACATTTGGGGGACATGATGCAAAGTTTATAGATAGTTTACAGGAAAACGAATT |
| | | | TCGTCTATATTATTATAATAAGTTTAAAGATATAGCAAGTACACTTAATAAAGCTAAATCAATAG |
| | | | TAGGTACTACTGCTTCATTACAGTATATGAAAAATGTTTTTAAAGAGAAATATCTCCTATCTGAA |
| | | | GATACATCTGGAAAATTTTCGGTAGATAAATTAAAATTTGATAAGTTATACAAAATGTTAACAGA |
| | | | GATTTACACAGAGGATAATTTTGTTAAGTTTTTTAAAGTACTTAACAGAAAAACATATTTGAATT |
| | | | TTGATAAAGCCGTATTTAAGATAAATATAGTACCTAAGGTAAATTACACAATATATGATGGATTT |
| | | | AATTTAAGAAATACAAATTTAGCAGCAAACTTTAATGGTCAAAATACAGAAATTAATAATATGAA |
| | | | TTTTACTAAACTAAAAAATTTTACTGGATTGTTTGAATTTTATAAGTTGCTATGTGTGCGCGGCA |
| | | | TCATTACCCTGGGGCAGATCCAGCTGACCATCCGGCACAGCTCGCAGAGAAACAAGCTTATCGTG |
| | | | GTCGTGCATGCCTGCAGAAACCTCATTGCCTTCTCTGAAGACGGCTCTGACCCCTATGTCCGCAT |
| | | | GTATTTATTACCAGACAAGAGGCGGTCAGGAAGGAGGAAAACACACGTGTCAAAGAAAACATTAA |
| | | | ATCCAGTGTTTGATCAAAGCTTTGATTTCAGTGTTTCGTTACCAGAAGTGCAGAGGAGAACGCTC |
| | | | GACGTTGCCGTGAAGAACAGTGGCGGCTTCCTGTCCAAAGACAAAGGGCTCCTTGGCAAAGTATT |
| | | | GGTTGCTCTGGCATCTGAAGAACTTGCCAAAGGCTGGACCCAGTGGTATGACCTCACGGAAGATG |
| | | | GGACGAGCAAGGCAGGTGCGGGCAAGTCCTTGGTTCCGCGTGGCAGCGCCGGCGCCGGCGCGCTC |
| | | | AATGATCTGTGTATCAAAGTTAATAATTGGGACTTGTTTTTTAGTCCTTCAGAAGATAATTTTAC |
| | | | TAATGATCTAAATAAAGGAGAAGAAATTACATCTGATACTAATATAGAAGCAGCAGAAGAAAATA |
| | | | TTAGTTTAGATTTAATACAACAATATTATTTAACCTTTAATTTTGATAATGAACCTGAAAATATT |
| | | | TCAATAGAAAATCTTTCAAGTGACATTATAGGCCAATTAGAACTTATGCCTAATATAGAAAGATT |
| | | | TCCTAATGGAAAAAAGTATGAGTTAGATAAATATACTATGTTCCATTATCTTCGTGCTCAAGAAT |
| | | | TTGAACATGGTAAATCTAGGATTGCTTTAACAAATTCTGTTAACGAAGCATTATTAAATCCTAGT |
| | | | CGTGTTTATACATTTTTTTCTTCAGACTATGTAAAGAAAGTTAATAAAGCTACGGAGGCAGCTAT |
| | | | GTTTTTAGGCTGGGTAGAACAATTAGTATATGATTTTACCGATGAAACTAGCGAAGTAAGTACTA |
| | | | CGGATAAAATTGCGGATATAACTATAATTATTCCATATATAGGACCTGCTTTAAATATAGGTAAT |
| | | | ATGTTATATAAAGATGATTTTGTAGGTGCTTTAATATTTTCAGGAGCTGTTATTCTGTTAGAATT |
| | | | TATACCAGAGATTGCAATACCTGTATTAGGTACTTTTGCACTTGTATCATATATTGCGAATAAGG |
| | | | TTCTAACCGTTCAAACAATAGATAATGCTTTAAGTAAAAGAAATGAAAAATGGGATGAGGTCTAT |
| | | | AAATATATAGTAACAAATTGGTTAGCAAAGGTTAATACACAGATTGATCTAATAAGAAAAAAAAT |
| | | | GAAAGAAGCTTTAGAAAATCAAGCAGAAGCAACAAAGGCTATAATAAACTATCAGTATAATCAAT |
| | | | ATACTGAGGAAGAGAAAAATAATATTAATTTTAATATTGATGATTTAAGTTCGAAACTTAATGAG |
| | | | TCTATAAATAAAGCTATGATTAATATAAATAAATTTTTGAATCAATGCTCTGTTTCATATTTAAT |
| | | | GAATTCTATGATCCCTTATGGTGTTAAACGGTTAGAAGATTTTGATGCTAGTCTTAAAGATGCAT |
| | | | TATTAAAGTATATATATGATAATAGAGGAACTTTAATTGGTCAAGTAGATAGATTAAAAGATAAA |
| | | | GTTAATAATACACTTAGTACAGATATACCTTTTCAGCTTTCCAAATACGTAGATAATCAAAGATT |
| | | | ATTATCTACATTTACTGAATATATTAAGAATATTATTAATACTTCTATATTGAATTTAAGATATG |
| | | | AAAGTAATCATTTAATAGACTTATCTAGGTATGCATCAAAAATAAATATTGGTAGTAAAGTAAAT |
| | | | TTTGATCCAATAGATAAAAATCAAATTCAATTATTTAATTTAGAAAGTAGTAAAATTGAGGTAAT |
| | | | TTTAAAAAATGCTATTGTATATAATAGTATGTATGAAAATTTTAGTACTAGCTTTTGGATAAGAA |
| | | | TTCCTAAGTATTTTAACAGTATAAGTCTAAATAATGAATATACAATAATAAATTGTATGGAAAAT |
| | | | AATTCAGGATGGAAAGTATCACTTAATTATGGTGAAATAATCTGGACTTTACAGGATACTCAGGA |
| | | | AATAAAACAAAGAGTAGTTTTTAAATACAGTCAAATGATTAATATATCAGATTATATAAACAGAT |
| | | | GGATTTTTGTAACTATCACTAATAATAGATTAAATAACTCTAAAATTTATATAAATGGAAGATTA |
| | | | ATAGATCAAAAACCAATTTCAAATTTAGGTAATATTCATGCTAGTAATAATATAATGTTTAAATT |
| | | | AGATGGTTGTAGAGATACACATAGATATATTTGGATAAAATATTTTAATCTTTTTGATAAGGAAT |
| | | | TAAATGAAAAAGAAATCAAAGATTTATATGATAATCAATCAAATTCAGGTATTTTAAAAGACTTT |
| | | | TGGGGTGATTATTTACAATATGATAAACCATACTATATGTTAAATTTATATGATCCAAATAAATA |
| | | | TGTCGATGTAAATAATGTAGGTATTAGAGGTTATATGTATCTTAAAGGGCCTAGAGGTAGCGTAA |
| | | | TGACTACAAACATTTATTTAAATTCAAGTTTGTATAGGGGGACAAAATTTATTATAAAAAAATAT |
| | | | GCTTCTGGAAATAAAGATAATATTGTTAGAAATAATGATCGTGTATATATTAATGTAGTAGTTAA |
| | | | AAATAAAGAATATAGGTTAGCTACTAATGCATCACAGGCAGGCGTAGAAAAAATACTAAGTGCAT |
| | | | TAGAAATACCTGATGTAGGAAATCTAAGTCAAGTAGTAGTAATGAAGTCAAAAAATGATCAAGGA |
| | | | ATAACAAATAAATGCAAAATGAATTTACAAGATAATAATGGGAATGATATAGGCTTTATAGGATT |
| | | | TCATCAGTTTAATAATATAGCTAAACTAGTAGCAAGTAATTGGTATAATAGACAAATAGAAAGAT |
| | | | CTAGTAGGACTTTGGGTTGCTCATGGGAATTTATTCCTGTAGATGATGGATGGGGAGAAAGGCCA |
| | | | CTGTAA |

TABLE 5
C2 Domain-Containing Proteins Listed in UniProtKB/Swiss-Prot (Reviewed Proteins)
[Domain(s): C2 domain(s) = C2 calcium-dependent membrane targeting domain(s);
number in parentheses = number of C2 domains in protein]

| Entry | Entry name | Domain(s) | Entry | Entry name | Domain(s) | Entry | Entry name | Domain(s) |
|---|---|---|---|---|---|---|---|---|
| P34885 | KPC1B_CAEEL | C2 domain (1) | Q16974 | KPC1_APLCA | C2 domain (1) | P05130 | KPC1_DROME | C2 domain (1) |
| Q25378 | KPC1_LYTPI | C2 domain (1) | Q16975 | KPC2_APLCA | C2 domain (1) | P90980 | KPC2_CAEEL | C2 domain (1) |
| P13677 | KPC2_DROME | C2 domain (1) | P04409 | KPCA_BOVIN | C2 domain (1) | P17252 | KPCA_HUMAN | C2 domain (1) |
| P20444 | KPCA_MOUSE | C2 domain (1) | P10102 | KPCA_RABIT | C2 domain (1) | P05696 | KPCA_RAT | C2 domain (1) |
| P05126 | KPCB_BOVIN | C2 domain (1) | Q7SY24 | KPCB_DANRE | C2 domain (1) | P05771 | KPCB_HUMAN | C2 domain (1) |
| P68404 | KPCB_MOUSE | C2 domain (1) | P05772 | KPCB_RABIT | C2 domain (1) | P68403 | KPCB_RAT | C2 domain (1) |
| Q7LZQ8 | KPCB_XENLA | C2 domain (1) | A8KBH6 | KPCB_XENTR | C2 domain (1) | Q5PU49 | KPCD_CANFA | C2 domain (1) |
| Q05655 | KPCD_HUMAN | C2 domain (1) | P28867 | KPCD_MOUSE | C2 domain (1) | P09215 | KPCD_RAT | C2 domain (1) |
| Q02156 | KPCE_HUMAN | C2 domain (1) | P16054 | KPCE_MOUSE | C2 domain (1) | P10830 | KPCE_RABIT | C2 domain (1) |
| P09216 | KPCE_RAT | C2 domain (1) | P05128 | KPCG_BOVIN | C2 domain (1) | P05129 | KPCG_HUMAN | C2 domain (1) |
| Q4R4U2 | KPCG_MACFA | C2 domain (1) | P63318 | KPCG_MOUSE | C2 domain (1) | P10829 | KPCG_RABIT | C2 domain (1) |
| P63319 | KPCG_RAT | C2 domain (1) | P24723 | KPCL_HUMAN | C2 domain (1) | P23298 | KPCL_MOUSE | C2 domain (1) |
| Q64617 | KPCL_RAT | C2 domain (1) | Q04759 | KPCT_HUMAN | C2 domain (1) | Q02111 | KPCT_MOUSE | C2 domain (1) |
| Q9WTQ0 | KPCT_RAT | C2 domain (1) | P24583 | KPC1_YEAST | C2 domain (1) | P11792 | SCH9_YEAST | C2 domain (1) |
| Q54IF2 | Y0670_DICDI | C2 domain (1) | A1A4I4 | PKN1_BOVIN | C2 domain (1) | Q16512 | PKN1_HUMAN | C2 domain (1) |
| P70268 | PKN1_MOUSE | C2 domain (1) | Q63433 | PKN1_RAT | C2 domain (1) | A7MBL8 | PKN2_DANRE | C2 domain (1) |
| Q16513 | PKN2_HUMAN | C2 domain (1) | Q8BWW9 | PKN2_MOUSE | C2 domain (1) | O08874 | PKN2_RAT | C2 domain (1) |
| A1Z7T0 | PKN_DROME | C2 domain (1) | Q55A55 | Y9848_DICDI | C2 domain (1) | Q8L7A4 | AGD11_ARATH | C2 domain (1) |
| Q9FVJ3 | AGD12_ARATH | C2 domain (1) | Q8LFN9 | AGD13_ARATH | C2 domain (1) | P48423 | GAP1_DROME | C2 domain (2) |
| C9J798 | RAS4B_HUMAN | C2 domain (2) | Q15283 | RASA2_HUMAN | C2 domain (2) | P58069 | RASA2_MOUSE | C2 domain (2) |
| Q63713 | RASA2_RAT | C2 domain (2) | Q28013 | RASA3_BOVIN | C2 domain (2) | Q14644 | RASA3_HUMAN | C2 domain (2) |
| Q60790 | RASA3_MOUSE | C2 domain (2) | Q9QYJ2 | RASA3_RAT | C2 domain (2) | O95294 | RASL1_HUMAN | C2 domain (2) |
| Q9Z268 | RASL1_MOUSE | C2 domain (2) | O43374 | RASL2_HUMAN | C2 domain (2) | Q6PFQ7 | RASL2_MOUSE | C2 domain (2) |
| Q86KB1 | ADCB_DICDI | C2 domain (1) | Q55CH0 | ADCC_DICDI | C2 domain (1) | Q941L2 | BAP1_ARATH | C2 domain (1) |
| Q58FX0 | BAP2_ARATH | C2 domain (1) | Q2KJ18 | C2C4A_BOVIN | C2 domain (1) | Q8NCU7 | C2C4A_HUMAN | C2 domain (1) |
| A6NLJ0 | C2C4B_HUMAN | C2 domain (1) | Q8TF44 | C2C4C_HUMAN | C2 domain (1) | Q5HZI2 | C2C4C_MOUSE | C2 domain (1) |
| P0CG09 | C2C4D_MOUSE | C2 domain (1) | Q6P1N0 | C2D1A_HUMAN | C2 domain (1) | Q8K1A6 | C2D1A_MOUSE | C2 domain (1) |
| Q66HA5 | C2D1A_RAT | C2 domain (1) | Q5T0F9 | C2D1B_HUMAN | C2 domain (1) | Q8BRN9 | C2D1B_MOUSE | C2 domain (1) |
| Q5FVK6 | C2D1B_RAT | C2 domain (1) | Q6PF54 | C2D1B_XENLA | C2 domain (1) | Q9U2M8 | C2D1_CAEEL | C2 domain (1) |
| Q9VKJ9 | C2D1_DROME | C2 domain (1) | Q29M42 | C2D1_DROPS | C2 domain (1) | Q9P2K1 | C2D2A_HUMAN | C2 domain (1) |
| Q8CFW7 | C2D2A_MOUSE | C2 domain (1) | B7Z1M9 | C2D4D_HUMAN | C2 domain (1) | Q93XX4 | C2D61_ARATH | C2 domain (1) |
| Q8N5R6 | CCD33_HUMAN | C2 domain (1) | Q3ULW6 | CCD33_MOUSE | C2 domain (1) | Q5XIR4 | CCD33_RAT | C2 domain (1) |
| Q8MUF9 | CPLA_DICDI | C2 domain (1) | Q9Y426 | CU025_HUMAN | C2 domain (1) | A2WWV5 | ERG1_ORYSI | C2 domain (1) |
| Q0JHU5 | ERG1_ORYSJ | C2 domain (1) | Q25AG5 | ERG3_ORYSI | C2 domain (1) | Q0JBH9 | ERG3_ORYSJ | C2 domain (1) |
| O94701 | INN1_SCHPO | C2 domain (1) | P53901 | INN1_YEAST | C2 domain (1) | Q96PE3 | INP4A_HUMAN | C2 domain (1) |
| Q9EPW0 | INP4A_MOUSE | C2 domain (1) | Q62784 | INP4A_RAT | C2 domain (1) | O15327 | INP4B_HUMAN | C2 domain (1) |
| Q4R4D7 | INP4B_MACFA | C2 domain (1) | Q6P1Y8 | INP4B_MOUSE | C2 domain (1) | Q5RA60 | INP4B_PONAB | C2 domain (1) |
| Q9QWG5 | INP4B_RAT | C2 domain (1) | Q86YS7 | K0528_HUMAN | C2 domain (1) | Q7TPS5 | K0528_MOUSE | C2 domain (1) |
| Q5RDC8 | K0528_PONAB | C2 domain (1) | Q28BX9 | K0528_XENTR | C2 domain (1) | Q9ZT47 | PP16A_CUCMA | C2 domain (1) |
| Q9ZT46 | PP16B_CUCMA | C2 domain (1) | P53037 | PSD2_YEAST | C2 domain (1) | O14111 | PSD3_SCHPO | C2 domain (1) |
| Q9UJD0 | RIMS3_HUMAN | C2 domain (1) | Q80U57 | RIMS3_MOUSE | C2 domain (1) | Q9JIR3 | RIMS3_RAT | C2 domain (1) |
| Q9H426 | RIMS4_HUMAN | C2 domain (1) | P60191 | RIMS4_MOUSE | C2 domain (1) | Q8CIX1 | RIMS4_RAT | C2 domain (1) |
| Q9GLM3 | RPGR1_BOVIN | C2 domain (1) | Q96KN7 | RPGR1_HUMAN | C2 domain (1) | Q9EPQ2 | RPGR1_MOUSE | C2 domain (1) |
| Q58G82 | SY14L_HUMAN | C2 domain (1) | Q8N9U0 | TAC2N_HUMAN | C2 domain (1) | Q91XT6 | TAC2N_MOUSE | C2 domain (1) |
| Q9P2Y5 | UVRAG_HUMAN | C2 domain (1) | Q9ULE0 | WWC3_HUMAN | C2 domain (1) | Q54FM6 | Y0753_DICDI | C2 domain (1) |
| Q9C8S6 | Y1322_ARATH | C2 domain (1) | O00443 | P3C2A_HUMAN | C2 domain (1) | Q61194 | P3C2A_MOUSE | C2 domain (1) |
| Q5RAY1 | P3C2A_PONAB | C2 domain (1) | O00750 | P3C2B_HUMAN | C2 domain (1) | O75747 | P3C2G_HUMAN | C2 domain (1) |
| O70167 | P3C2G_MOUSE | C2 domain (1) | O70173 | P3C2G_RAT | C2 domain (1) | Q22036 | CAN5_CAEEL | C2 domain (1) |
| O15484 | CAN5_HUMAN | C2 domain (1) | O08688 | CAN5_MOUSE | C2 domain (1) | Q8R4C0 | CAN5_RAT | C2 domain (1) |
| Q9Y6Q1 | CAN6_HUMAN | C2 domain (1) | O35646 | CAN6_MOUSE | C2 domain (1) | O88501 | CAN6_RAT | C2 domain (1) |
| Q4LBC8 | TOIPA_ONCMY | C2 domain (1) | Q3B8H2 | TOIPA_XENLA | C2 domain (1) | Q4LBC7 | TOIPB_ONCMY | C2 domain (1) |
| Q6INE3 | TOIPB_XENLA | C2 domain (1) | Q2LGB5 | TOLIP_BOVIN | C2 domain (1) | Q5ZK05 | TOLIP_CHICK | C2 domain (1) |
| Q7ZV43 | TOLIP_DANRE | C2 domain (1) | C1BZR1 | TOLIP_ESOLU | C2 domain (1) | Q9H0E2 | TOLIP_HUMAN | C2 domain (1) |
| Q9QZ06 | TOLIP_MOUSE | C2 domain (1) | A2RUW1 | TOLIP_RAT | C2 domain (1) | B5X370 | TOLIP_SALSA | C2 domain (1) |
| Q6DFR0 | TOLIP_XENTR | C2 domain (1) | Q54WH2 | FORA_DICDI | C2 domain (1) | Q15811 | ITSN1_HUMAN | C2 domain (1) |
| Q9Z0R4 | ITSN1_MOUSE | C2 domain (1) | Q9NZM3 | ITSN2_HUMAN | C2 domain (1) | Q9Z0R6 | ITSN2_MOUSE | C2 domain (1) |
| A6QNS3 | ABR_BOVIN | C2 domain (1) | Q12979 | ABR_HUMAN | C2 domain (1) | Q5SSL4 | ABR_MOUSE | C2 domain (1) |
| Q8AVG0 | ABR_XENLA | C2 domain (1) | A4II46 | ABR_XENTR | C2 domain (1) | P11274 | BCR_HUMAN | C2 domain (1) |
| Q6PAJ1 | BCR_MOUSE | C2 domain (1) | P08487 | PLCG1_BOVIN | C2 domain (1) | P19174 | PLCG1_HUMAN | C2 domain (1) |
| Q62077 | PLCG1_MOUSE | C2 domain (1) | P10686 | PLCG1_RAT | C2 domain (1) | P40977 | PLC1_SCHPO | C2 domain (1) |
| P32383 | PLC1_YEAST | C2 domain (1) | Q39032 | PLCD1_ARATH | C2 domain (1) | Q56W08 | PLCD3_ARATH | C2 domain (1) |
| Q944C1 | PLCD4_ARATH | C2 domain (1) | Q1RML2 | PLCZ1_BOVIN | C2 domain (1) | Q2VRL0 | PLCZ1_CHICK | C2 domain (1) |
| Q86YW0 | PLCZ1_HUMAN | C2 domain (1) | Q95JS1 | PLCZ1_MACFA | C2 domain (1) | Q8K4D7 | PLCZ1_MOUSE | C2 domain (1) |
| Q7YRU3 | PLCZ1_PIG | C2 domain (1) | Q5FX52 | PLCZ1_RAT | C2 domain (1) | Q02158 | PLC_DICDI | C2 domain (1) |
| P10895 | PLCD1_BOVIN | C2 domain (1) | P51178 | PLCD1_HUMAN | C2 domain (1) | Q8R3B1 | PLCD1_MOUSE | C2 domain (1) |
| P10688 | PLCD1_RAT | C2 domain (1) | Q4KWH8 | PLCH1_HUMAN | C2 domain (1) | Q4KWH5 | PLCH1_MOUSE | C2 domain (1) |
| O75038 | PLCH2_HUMAN | C2 domain (1) | A2AP18 | PLCH2_MOUSE | C2 domain (1) | Q8N3E9 | PLCD3_HUMAN | C2 domain (1) |
| Q8K2J0 | PLCD3_MOUSE | C2 domain (1) | P21671 | PLCD4_BOVIN | C2 domain (1) | Q9BRC7 | PLCD4_HUMAN | C2 domain (1) |
| Q4R6L3 | PLCD4_MACFA | C2 domain (1) | Q8K3R3 | PLCD4_MOUSE | C2 domain (1) | Q8SPR7 | PLCD4_PIG | C2 domain (1) |
| Q5RET0 | PLCD4_PONAB | C2 domain (1) | Q62711 | PLCD4_RAT | C2 domain (1) | Q32NH8 | PLCD4_XENLA | C2 domain (1) |
| A5D6R3 | PLD3A_DANRE | C2 domain (1) | Q39033 | PLCD2_ARATH | C2 domain (1) | Q944C2 | PLCD5_ARATH | C2 domain (1) |
| Q9LY51 | PLCD7_ARATH | C2 domain (1) | P14222 | PERF_HUMAN | C2 domain (1) | P10820 | PERF_MOUSE | C2 domain (1) |
| P35763 | PERF_RAT | C2 domain (1) | Q6WKZ4 | RFIP1_HUMAN | C2 domain (1) | Q9D620 | RFIP1_MOUSE | C2 domain (1) |
| Q3B7T9 | RFIP1_RAT | C2 domain (1) | Q7L804 | RFIP2_HUMAN | C2 domain (1) | Q9BXF6 | RFIP5_HUMAN | C2 domain (1) |
| Q8R361 | RFIP5_MOUSE | C2 domain (1) | Q9UTG2 | PUB2_SCHPO | C2 domain (1) | Q76N89 | HECW1_HUMAN | C2 domain (1) |
| Q8K4P8 | HECW1_MOUSE | C2 domain (1) | Q9P2P5 | HECW2_HUMAN | C2 domain (1) | Q6I6G8 | HECW2_MOUSE | C2 domain (1) |
| Q9HCE7 | SMUF1_HUMAN | C2 domain (1) | Q9CUN6 | SMUF1_MOUSE | C2 domain (1) | Q9PUN2 | SMUF1_XENLA | C2 domain (1) |
| Q9VVI3 | NEDD4_DROME | C2 domain (1) | P46935 | NEDD4_MOUSE | C2 domain (1) | Q62940 | NEDD4_RAT | C2 domain (1) |
| Q92462 | PUB1_SCHPO | C2 domain (1) | O14326 | PUB3_SCHPO | C2 domain (1) | A1CQG2 | RSP5_ASPCL | C2 domain (1) |
| B0XQ72 | RSP5_ASPFC | C2 domain (1) | B8N7E5 | RSP5_ASPFN | C2 domain (1) | Q4WTF3 | RSP5_ASPFU | C2 domain (1) |
| A2QQ28 | RSP5_ASPNC | C2 domain (1) | Q2UBP1 | RSP5_ASPOR | C2 domain (1) | Q0CCL1 | RSP5_ASPTN | C2 domain (1) |
| Q5BDP1 | RSP5_EMENI | C2 domain (1) | A1D3C5 | RSP5_NEOFI | C2 domain (1) | P39940 | RSP5_YEAST | C2 domain (1) |
| Q9V853 | SMUF1_DROME | C2 domain (1) | A9JRZ0 | SMUF2_DANRE | C2 domain (1) | Q9HAU4 | SMUF2_HUMAN | C2 domain (1) |
| A2A5Z6 | SMUF2_MOUSE | C2 domain (1) | Q2TAS2 | SMUF2_XENLA | C2 domain (1) | Q96J02 | ITCH_HUMAN | C2 domain (1) |
| Q8C863 | ITCH_MOUSE | C2 domain (1) | Q96PU5 | NED4L_HUMAN | C2 domain (1) | Q8CFI0 | NED4L_MOUSE | C2 domain (1) |
| Q5RBF2 | NED4L_PONAB | C2 domain (1) | Q9Y0H4 | SUDX_DROME | C2 domain (1) | Q9H0M0 | WWP1_HUMAN | C2 domain (1) |
| Q8BZZ3 | WWP1_MOUSE | C2 domain (1) | O00308 | WWP2_HUMAN | C2 domain (1) | Q9DBH0 | WWP2_MOUSE | C2 domain (1) |
| O13683 | YDY2_SCHPO | C2 domain (1) | Q08748 | YO296_YEAST | C2 domain (1) | Q9ULU8 | CAPS1_HUMAN | C2 domain (1) |
| Q80TJ1 | CAPS1_MOUSE | C2 domain (1) | Q62717 | CAPS1_RAT | C2 domain (1) | Q6GLR7 | CAPS1_XENLA | C2 domain (1) |
| Q86UW7 | CAPS2_HUMAN | C2 domain (1) | Q8BYR5 | CAPS2_MOUSE | C2 domain (1) | Q60PC0 | CAPS_CAEBR | C2 domain (1) |
| Q23658 | CAPS_CAEEL | C2 domain (1) | Q9NHE5 | CAPS_DROME | C2 domain (1) | P49796 | RGS3_HUMAN | C2 domain (1) |
| Q86NH1 | SYD1_CAEEL | C2 domain (1) | Q9V9S7 | SYDE_DROME | C2 domain (1) | Q15111 | PLCL1_HUMAN | C2 domain (1) |
| Q3USB7 | PLCL1_MOUSE | C2 domain (1) | Q62688 | PLCL1_RAT | C2 domain (1) | Q9UPR0 | PLCL2_HUMAN | C2 domain (1) |
| Q8K394 | PLCL2_MOUSE | C2 domain (1) | P16885 | PLCG2_HUMAN | C2 domain (1) | Q8CIH5 | PLCG2_MOUSE | C2 domain (1) |
| P24135 | PLCG2_RAT | C2 domain (1) | Q5VWQ8 | DAB2P_HUMAN | C2 domain (1) | Q3UHC7 | DAB2P_MOUSE | C2 domain (1) |
| Q8MLZ5 | GAP2_CAEEL | C2 domain (1) | Q8T498 | GAP2_DROME | C2 domain (1) | Q9UJF2 | NGAP_HUMAN | C2 domain (1) |
| A6QQ91 | RASL3_BOVIN | C2 domain (1) | Q86YV0 | RASL3_HUMAN | C2 domain (1) | Q8C2K5 | RASL3_MOUSE | C2 domain (1) |
| Q96PV0 | SYGP1_HUMAN | C2 domain (1) | F6SEU4 | SYGP1_MOUSE | C2 domain (1) | Q9QUH6 | SYGP1_RAT | C2 domain (1) |
| P09851 | RASA1_BOVIN | C2 domain (1) | P20936 | RASA1_HUMAN | C2 domain (1) | P50904 | RASA1_RAT | C2 domain (1) |
| Q54E35 | GACEE_DICDI | C2 domain (1) | P25455 | PIP1_DROME | C2 domain (1) | P13217 | PIPA_DROME | C2 domain (1) |
| O13433 | PLC1_CANAX | C2 domain (1) | P10894 | PLCB1_BOVIN | C2 domain (1) | Q9NQ66 | PLCB1_HUMAN | C2 domain (1) |
| Q9Z1B3 | PLCB1_MOUSE | C2 domain (1) | P10687 | PLCB1_RAT | C2 domain (1) | Q00722 | PLCB2_HUMAN | C2 domain (1) |
| A3KGF7 | PLCB2_MOUSE | C2 domain (1) | O89040 | PLCB2_RAT | C2 domain (1) | Q01970 | PLCB3_HUMAN | C2 domain (1) |
| P51432 | PLCB3_MOUSE | C2 domain (1) | Q99JE6 | PLCB3_RAT | C2 domain (1) | Q07722 | PLCB4_BOVIN | C2 domain (1) |
| Q15147 | PLCB4_HUMAN | C2 domain (1) | Q9QW07 | PLCB4_RAT | C2 domain (1) | Q8GV43 | PLCD6_ARATH | C2 domain (1) |
| Q9STZ3 | PLCD8_ARATH | C2 domain (1) | Q6NMA7 | PLCD9_ARATH | C2 domain (1) | Q9P212 | PLCE1_HUMAN | C2 domain (1) |
| Q8K4S1 | PLCE1_MOUSE | C2 domain (1) | Q99P84 | PLCE1_RAT | C2 domain (1) | A4IFJ5 | PA24A_BOVIN | C2 domain (1) |
| P49147 | PA24A_CHICK | C2 domain (1) | P50392 | PA24A_DANRE | C2 domain (1) | O77793 | PA24A_HORSE | C2 domain (1) |
| P47712 | PA24A_HUMAN | C2 domain (1) | P47713 | PA24A_MOUSE | C2 domain (1) | Q5R8A5 | PA24A_PONAB | C2 domain (1) |
| Q9TT38 | PA24A_RABIT | C2 domain (1) | P50393 | PA24A_RAT | C2 domain (1) | Q7T0T9 | PA24A_XENLA | C2 domain (1) |
| B1WAZ6 | PA24A_XENTR | C2 domain (1) | P0C869 | PA24B_HUMAN | C2 domain (1) | P0C871 | PA24B_MOUSE | C2 domain (1) |
| Q86XP0 | PA24D_HUMAN | C2 domain (1) | Q50L43 | PA24D_MOUSE | C2 domain (1) | Q3MJ16 | PA24E_HUMAN | C2 domain (1) |
| Q50L42 | PA24E_MOUSE | C2 domain (1) | Q68DD2 | PA24F_HUMAN | C2 domain (1) | Q50L41 | PA24F_MOUSE | C2 domain (1) |
| Q38882 | PLDA1_ARATH | C2 domain (1) | O82549 | PLDA1_BRAOC | C2 domain (1) | P86387 | PLDA1_CARPA | C2 domain (1) |
| Q70EW5 | PLDA1_CYNCA | C2 domain (1) | Q43270 | PLDA1_MAIZE | C2 domain (1) | Q43007 | PLDA1_ORYSJ | C2 domain (1) |
| O04883 | PLDA1_PIMBR | C2 domain (1) | Q41142 | PLDA1_RICCO | C2 domain (1) | P93400 | PLDA1_TOBAC | C2 domain (1) |
| O04865 | PLDA1_VIGUN | C2 domain (1) | Q9SSQ9 | PLDA2_ARATH | C2 domain (1) | P55939 | PLDA2_BRAOC | C2 domain (1) |
| P93844 | PLDA2_ORYSJ | C2 domain (1) | P93733 | PLDB1_ARATH | C2 domain (1) | O23078 | PLDB2_ARATH | C2 domain (1) |
| Q9C5Y0 | PLDD1_ARATH | C2 domain (1) | Q9C888 | PLDE1_ARATH | C2 domain (1) | Q9T053 | PLDG1_ARATH | C2 domain (1) |
| Q9T051 | PLDG2_ARATH | C2 domain (1) | Q9T052 | PLDG3_ARATH | C2 domain (1) | P58766 | PLDZ1_ARATH | C2 domain (1) |
| P33314 | BUD2_YEAST | C2 domain (1) | Q6P730 | DAB2P_RAT | C2 domain (1) | Q54Y08 | NGAP_DICDI | C2 domain (1) |
| Q8WQC0 | RGS7_CAEEL | C2 domain (1) | Q61CA4 | SYD1_CAEBR | C2 domain (1) | Q5VT97 | SYDE2_HUMAN | C2 domain (1) |
| Q54T86 | DWWA_DICDI | C2 domain (1) | B3LWS4 | KIBRA_DROAN | C2 domain (1) | B3P3M8 | KIBRA_DROER | C2 domain (1) |
| Q9VFG8 | KIBRA_DROME | C2 domain (1) | B4K6I9 | KIBRA_DROMO | C2 domain (1) | B4HEJ6 | KIBRA_DROSE | C2 domain (1) |
| B4M5X4 | KIBRA_DROVI | C2 domain (1) | B4NAD3 | KIBRA_DROWI | C2 domain (1) | B4PSQ2 | KIBRA_DROYA | C2 domain (1) |
| Q8IX03 | KIBRA_HUMAN | C2 domain (1) | Q5SXA9 | KIBRA_MOUSE | C2 domain (1) | A4IIJ3 | KIBRA_XENTR | C2 domain (1) |
| Q6AWC2 | WWC2_HUMAN | C2 domain (1) | Q6NXJ0 | WWC2_MOUSE | C2 domain (1) | Q6DJR2 | WWC2_XENTR | C2 domain (1) |
| Q4AC94 | C2CD3_HUMAN | C2 domains (2) | Q52KB6 | C2CD3_MOUSE | C2 domains (2) | A0JM13 | C2CD3_XENTR | C2 domains (2) |
| Q14183 | DOC2A_HUMAN | C2 domains (2) | Q7TNF0 | DOC2A_MOUSE | C2 domains (2) | P70611 | DOC2A_RAT | C2 domains (2) |
| Q14184 | DOC2B_HUMAN | C2 domains (2) | P70169 | DOC2B_MOUSE | C2 domains (2) | P70610 | DOC2B_RAT | C2 domains (2) |
| Q9ESN1 | DOC2G_MOUSE | C2 domains (2) | Q17388 | FER1_CAEEL | C2 domains (2) | Q68CZ1 | FTM_HUMAN | C2 domains (2) |
| Q8CG73 | FTM_MOUSE | C2 domains (2) | Q9USG8 | MU190_SCHPO | C2 domains (2) | P24505 | SY61_DISOM | C2 domains (2) |
| P24506 | SY62_DISOM | C2 domains (2) | P24507 | SY63_DISOM | C2 domains (2) | P41823 | SY65_APLCA | C2 domains (2) |
| P21521 | SY65_DROME | C2 domains (2) | Q6XYQ8 | SYT10_HUMAN | C2 domains (2) | Q9R0N4 | SYT10_MOUSE | C2 domains (2) |
| Q5RCK6 | SYT10_PONAB | C2 domains (2) | O08625 | SYT10_RAT | C2 domains (2) | Q9BT88 | SYT11_HUMAN | C2 domains (2) |
| Q9R0N3 | SYT11_MOUSE | C2 domains (2) | O08835 | SYT11_RAT | C2 domains (2) | Q8IV01 | SYT12_HUMAN | C2 domains (2) |
| Q920N7 | SYT12_MOUSE | C2 domains (2) | P97610 | SYT12_RAT | C2 domains (2) | Q7L8C5 | SYT13_HUMAN | C2 domains (2) |
| Q9EQT6 | SYT13_MOUSE | C2 domains (2) | Q925B5 | SYT13_RAT | C2 domains (2) | Q8NB59 | SYT14_HUMAN | C2 domains (2) |
| Q7TN84 | SYT14_MOUSE | C2 domains (2) | Q9BQS2 | SYT15_HUMAN | C2 domains (2) | Q8C6N3 | SYT15_MOUSE | C2 domains (2) |
| P59926 | SYT15_RAT | C2 domains (2) | Q17RD7 | SYT16_HUMAN | C2 domains (2) | Q7TN83 | SYT16_MOUSE | C2 domains (2) |
| Q9BSW7 | SYT17_HUMAN | C2 domains (2) | Q920M7 | SYT17_MOUSE | C2 domains (2) | Q5R8Q5 | SYT17_PONAB | C2 domains (2) |
| Q62807 | SYT17_RAT | C2 domains (2) | A4IJ05 | SYT17_XENTR | C2 domains (2) | Q9SKR2 | SYT1_ARATH | C2 domains (2) |
| P48018 | SYT1_BOVIN | C2 domains (2) | P34693 | SYT1_CAEEL | C2 domains (2) | P47191 | SYT1_CHICK | C2 domains (2) |
| P21579 | SYT1_HUMAN | C2 domains (2) | Q60HC0 | SYT1_MACFA | C2 domains (2) | P46096 | SYT1_MOUSE | C2 domains (2) |
| Q5R4J5 | SYT1_PONAB | C2 domains (2) | P21707 | SYT1_RAT | C2 domains (2) | B6ETT4 | SYT2_ARATH | C2 domains (2) |
| Q8N9I0 | SYT2_HUMAN | C2 domains (2) | P46097 | SYT2_MOUSE | C2 domains (2) | P29101 | SYT2_RAT | C2 domains (2) |
| Q7XA06 | SYT3_ARATH | C2 domains (2) | Q9BQG1 | SYT3_HUMAN | C2 domains (2) | O35681 | SYT3_MOUSE | C2 domains (2) |
| P40748 | SYT3_RAT | C2 domains (2) | A0JJX5 | SYT4_ARATH | C2 domains (2) | Q9H2B2 | SYT4_HUMAN | C2 domains (2) |
| P40749 | SYT4_MOUSE | C2 domains (2) | P50232 | SYT4_RAT | C2 domains (2) | Q8L706 | SYT5_ARATH | C2 domains (2) |
| O00445 | SYT5_HUMAN | C2 domains (2) | Q9R0N5 | SYT5_MOUSE | C2 domains (2) | P47861 | SYT5_RAT | C2 domains (2) |
| Q5T7P8 | SYT6_HUMAN | C2 domains (2) | Q9R0N8 | SYT6_MOUSE | C2 domains (2) | Q62746 | SYT6_RAT | C2 domains (2) |
| O43581 | SYT7_HUMAN | C2 domains (2) | Q9R0N7 | SYT7_MOUSE | C2 domains (2) | Q8NBV8 | SYT8_HUMAN | C2 domains (2) |
| Q9R0N6 | SYT8_MOUSE | C2 domains (2) | Q925B4 | SYT8_RAT | C2 domains (2) | Q86SS6 | SYT9_HUMAN | C2 domains (2) |
| Q9R0N9 | SYT9_MOUSE | C2 domains (2) | Q925C0 | SYT9_RAT | C2 domains (2) | O14065 | YC31_SCHPO | C2 domains (2) |
| Q86UR5 | RIMS1_HUMAN | C2 domains (2) | Q99NE5 | RIMS1_MOUSE | C2 domains (2) | Q9JIR4 | RIMS1_RAT | C2 domains (2) |
| Q9UQ26 | RIMS2_HUMAN | C2 domains (2) | Q9EQZ7 | RIMS2_MOUSE | C2 domains (2) | Q9JIS1 | RIMS2_RAT | C2 domains (2) |
| Q22366 | RIM_CAEEL | C2 domains (2) | P41885 | RBF1_CAEEL | C2 domains (2) | Q06846 | RP3A_BOVIN | C2 domains (2) |
| Q9Y2J0 | RP3A_HUMAN | C2 domains (2) | P47708 | RP3A_MOUSE | C2 domains (2) | P47709 | RP3A_RAT | C2 domains (2) |
| Q96C24 | SYTL4_HUMAN | C2 domains (2) | Q9R0Q1 | SYTL4_MOUSE | C2 domains (2) | Q8VHQ7 | SYTL4_RAT | C2 domains (2) |
| Q8TDW5 | SYTL5_HUMAN | C2 domains (2) | Q80T23 | SYTL5_MOUSE | C2 domains (2) | Q812E4 | SYTL5_RAT | C2 domains (2) |
| Q9ZVT9 | C2GR1_ARATH | C2 domains (2) | Q9FGS8 | C2GR2_ARATH | C2 domains (2) | O94812 | BAIP3_HUMAN | C2 domains (2) |
| Q80TT2 | BAIP3_MOUSE | C2 domains (2) | Q70J99 | UN13D_HUMAN | C2 domains (2) | B2RUP2 | UN13D_MOUSE | C2 domains (2) |
| Q9R189 | UN13D_RAT | C2 domains (2) | Q8NB66 | UN13C_HUMAN | C2 domains (2) | Q8K0T7 | UN13C_MOUSE | C2 domains (2) |
| Q62770 | UN13C_RAT | C2 domains (2) | Q9PU36 | PCLO_CHICK | C2 domains (2) | Q9Y6V0 | PCLO_HUMAN | C2 domains (2) |
| Q9QYX7 | PCLO_MOUSE | C2 domains (2) | Q9JKS6 | PCLO_RAT | C2 domains (2) | Q8IYJ3 | SYTL1_HUMAN | C2 domains (2) |
| Q99N80 | SYTL1_MOUSE | C2 domains (2) | A6QP06 | SYTL2_BOVIN | C2 domains (2) | Q9HCH5 | SYTL2_HUMAN | C2 domains (2) |
| Q99N50 | SYTL2_MOUSE | C2 domains (2) | Q4VX76 | SYTL3_HUMAN | C2 domains (2) | Q99N48 | SYTL3_MOUSE | C2 domains (2) |
| Q941L3 | BON1_ARATH | C2 domains (2) | Q5S1W2 | BON2_ARATH | C2 domains (2) | Q5XQC7 | BON3_ARATH | C2 domains (2) |
| Q7YXU4 | CPNA_DICDI | C2 domains (2) | Q86K21 | CPNB_DICDI | C2 domains (2) | Q54P51 | CPNC_DICDI | C2 domains (2) |
| Q55GG1 | CPND_DICDI | C2 domains (2) | Q99829 | CPNE1_HUMAN | C2 domains (2) | Q8C166 | CPNE1_MOUSE | C2 domains (2) |
| Q96FN4 | CPNE2_HUMAN | C2 domains (2) | P59108 | CPNE2_MOUSE | C2 domains (2) | O75131 | CPNE3_HUMAN | C2 domains (2) |
| Q8BT60 | CPNE3_MOUSE | C2 domains (2) | Q5RAE1 | CPNE3_PONAB | C2 domains (2) | Q96A23 | CPNE4_HUMAN | C2 domains (2) |
| Q8BLR2 | CPNE4_MOUSE | C2 domains (2) | Q9HCH3 | CPNE5_HUMAN | C2 domains (2) | Q8JZW4 | CPNE5_MOUSE | C2 domains (2) |
| Q2KHY1 | CPNE6_BOVIN | C2 domains (2) | O95741 | CPNE6_HUMAN | C2 domains (2) | Q9Z140 | CPNE6_MOUSE | C2 domains (2) |
| Q5R4W6 | CPNE6_PONAB | C2 domains (2) | Q9UBL6 | CPNE7_HUMAN | C2 domains (2) | Q0VE82 | CPNE7_MOUSE | C2 domains (2) |
| Q86YQ8 | CPNE8_HUMAN | C2 domains (2) | Q9DC53 | CPNE8_MOUSE | C2 domains (2) | Q8IYJ1 | CPNE9_HUMAN | C2 domains (2) |
| Q1RLL3 | CPNE9_MOUSE | C2 domains (2) | Q5BJS7 | CPNE9_RAT | C2 domains (2) | Q54FY7 | CPNE_DICDI | C2 domains (2) |
| Q1ZXB3 | CPNF_DICDI | C2 domains (2) | A8WMY4 | NRA1_CAEBR | C2 domains (2) | Q9XUB9 | NRA1_CAEEL | C2 domains (2) |
| Q5FWL4 | EST2A_XENLA | C2 domains (3) | Q7ZWU7 | EST2B_XENLA | C2 domains (3) | A0FGR8 | ESYT2_HUMAN | C2 domains (3) |
| Q3TZZ7 | ESYT2_MOUSE | C2 domains (3) | A0FGR9 | ESYT3_HUMAN | C2 domains (3) | Q5DTI8 | ESYT3_MOUSE | C2 domains (3) |
| Q5M7N9 | ESYT3_XENTR | C2 domains (3) | Q6DN14 | MCTP1_HUMAN | C2 domains (3) | Q6DN12 | MCTP2_HUMAN | C2 domains (3) |
| Q5RJH2 | MCTP2_MOUSE | C2 domains (3) | Q12466 | TCB1_YEAST | C2 domains (3) | P48231 | TCB2_YEAST | C2 domains (3) |
| Q03640 | TCB3_YEAST | C2 domains (3) | Q9UT00 | YKH3_SCHPO | C2 domains (3) | Q9UPW8 | UN13A_HUMAN | C2 domains (3) |
| Q4KUS2 | UN13A_MOUSE | C2 domains (3) | Q62768 | UN13A_RAT | C2 domains (3) | O14795 | UN13B_HUMAN | C2 domains (3) |
| Q9Z1N9 | UN13B_MOUSE | C2 domains (3) | Q62769 | UN13B_RAT | C2 domains (3) | P27715 | UNC13_CAEEL | C2 domains (3) |
| B3DLH6 | MYOF_XENTR | C2 domains (4) | Q5SPC5 | OTOF_DANRE | C2 domains (4) | Q9HC10 | OTOF_HUMAN | C2 domains (4) |
| Q9ESF1 | OTOF_MOUSE | C2 domains (4) | Q9ERC5 | OTOF_RAT | C2 domains (4) | A6QQP7 | DYSF_BOVIN | C2 domains (5) |
| O75923 | DYSF_HUMAN | C2 domains (5) | Q9ESD7 | DYSF_MOUSE | C2 domains (5) | Q9BSJ8 | ESYT1_HUMAN | C2 domains (5) |
| Q3U7R1 | ESYT1_MOUSE | C2 domains (5) | Q5RAG2 | ESYT1_PONAB | C2 domains (5) | Q9Z1X1 | ESYT1_RAT | C2 domains (5) |
| A9Z1Z3 | FR1L4_HUMAN | C2 domains (5) | A3KGK3 | FR1L4_MOUSE | C2 domains (5) | Q9NZM1 | MYOF_HUMAN | C2 domains (5) |
| Q69ZN7 | MYOF_MOUSE | C2 domains (5) | A0AVI2 | FR1L5_HUMAN | C2 domains (6) | Q2WGJ9 | FR1L6_HUMAN | C2 domains (6) |
| P42347 | PI3K1_SOYBN | PI3K-type (1) | P42348 | PI3K2_SOYBN | PI3K-type (1) | P54676 | PI3K4_DICDI | PI3K-type (1) |
| P42339 | PI3K_ARATH | PI3K-type (1) | Q8NEB9 | PK3C3_HUMAN | PI3K-type (1) | Q6PF93 | PK3C3_MOUSE | PI3K-type (1) |
| Q5D891 | PK3C3_PIG | PI3K-type (1) | O88763 | PK3C3_RAT | PI3K-type (1) | Q6AZN6 | PK3C3_XENLA | PI3K-type (1) |
| Q92213 | VPS34_CANAX | PI3K-type (1) | P50520 | VPS34_SCHPO | PI3K-type (1) | P22543 | VPS34_YEAST | PI3K-type (1) |
| P0C5E7 | AGE1_CAEBR | PI3K-type (1) | Q94125 | AGE1_CAEEL | PI3K-type (1) | P54673 | PI3K1_DICDI | PI3K-type (1) |
| P32871 | PK3CA_BOVIN | PI3K-type (1) | P42336 | PK3CA_HUMAN | PI3K-type (1) | P42337 | PK3CA_MOUSE | PI3K-type (1) |
| P42338 | PK3CB_HUMAN | PI3K-type (1) | Q8BTI9 | PK3CB_MOUSE | PI3K-type (1) | Q9Z1L0 | PK3CB_RAT | PI3K-type (1) |
| O00329 | PK3CD_HUMAN | PI3K-type (1) | O35904 | PK3CD_MOUSE | PI3K-type (1) | P48736 | PK3CG_HUMAN | PI3K-type (1) |
| Q9JHG7 | PK3CG_MOUSE | PI3K-type (1) | O02697 | PK3CG_PIG | PI3K-type (1) | P54674 | PI3K2_DICDI | PI3K-type (1) |
| P54675 | PI3K3_DICDI | PI3K-type (1) | Q5T6R2 | TPT2L_HUMAN | tensin-type (1) | Q7XWS7 | FH12_ORYSJ | tensin-type (1) |
| Q9LVN1 | FH13_ARATH | tensin-type (1) | Q9C6S1 | FH14_ARATH | tensin-type (1) | Q9SK28 | FH18_ARATH | tensin-type (1) |
| Q9FLQ7 | FH20_ARATH | tensin-type (1) | Q7G6K7 | FH3_ORYSJ | tensin-type (1) | Q84ZL0 | FH5_ORYSJ | tensin-type (1) |
| Q6ZCX3 | FH6_ORYSJ | tensin-type (1) | Q6K8Z4 | FH7_ORYSJ | tensin-type (1) | Q27974 | AUXI_BOVIN | tensin-type (1) |
| O75061 | AUXI_HUMAN | tensin-type (1) | Q80TZ3 | AUXI_MOUSE | tensin-type (1) | O14976 | GAK_HUMAN | tensin-type (1) |
| Q99KY4 | GAK_MOUSE | tensin-type (1) | P97874 | GAK_RAT | tensin-type (1) | Q63HR2 | TENC1_HUMAN | tensin-type (1) |
| Q8CGB6 | TENC1_MOUSE | tensin-type (1) | Q54JL7 | CNRN_DICDI | tensin-type (1) | P60483 | PTEN_CANFA | tensin-type (1) |
| Q8T9S7 | PTEN_DICDI | tensin-type (1) | P60484 | PTEN_HUMAN | tensin-type (1) | O08586 | PTEN_MOUSE | tensin-type (1) |
| Q9PUT6 | PTEN_XENLA | tensin-type (1) | P91301 | TAG83_CAEEL | tensin-type (1) | Q6XPS3 | TPTE2_HUMAN | tensin-type (1) |
| Q4R6N0 | TPTE2_MACFA | tensin-type (1) | P56180 | TPTE_HUMAN | tensin-type (1) | Q9GLM4 | TENS1_BOVIN | tensin-type (1) |
| Q9HBL0 | TENS1_HUMAN | tensin-type (1) | Q68CZ2 | TENS3_HUMAN | tensin-type (1) | Q5SSZ5 | TENS3_MOUSE | tensin-type (1) |
| Q04205 | TENS_CHICK | tensin-type (1) | | | | | | |

Table 6: C2 Domain-Containing Human Proteins

TABLE 6A
C2 Domain-Containing Human Proteins Listed in UniProtKB/Swiss-Prot (Reviewed Proteins)
[Domains: C2 domain(s) = C2 calcium-dependent membrane targeting domain(s);;
number in parentheses = number of C2 domains in protein]

| | Entry name | | | Entry name | | | Entry name | |
|---|---|---|---|---|---|---|---|---|
| Entry | [. . . _HUMAN] | Domain(s) | Entry | [. . . _HUMAN] | Domain(s) | Entry | [. . . _HUMAN] | Domain(s) |
| Q12979 | ABR | C2 domain (1) | O94812 | BAIP3 | C2 domains (2) | P11274 | BCR | C2 domain (1) |
| Q8NCU7 | C2C4A | C2 domain (1) | A6NLJ0 | C2C4B | C2 domain (1) | Q8TF44 | C2C4C | C2 domain (1) |
| Q4AC94 | C2CD3 | C2 domains (2) | Q6P1N0 | C2D1A | C2 domain (1) | Q5T0F9 | C2D1B | C2 domain (1) |
| Q9P2K1 | C2D2A | C2 domain (1) | B7Z1M9 | C2D4D | C2 domain (1) | O15484 | CAN5 | C2 domain (1) |
| Q9Y6Q1 | CAN6 | C2 domain (1) | Q9ULU8 | CAPS1 | C2 domain (1) | Q86UW7 | CAPS2 | C2 domain (1) |
| Q8N5R6 | CCD33 | C2 domain (1) | Q99829 | CPNE1 | C2 domains (2) | Q96FN4 | CPNE2 | C2 domains (2) |
| O75131 | CPNE3 | C2 domains (2) | Q96A23 | CPNE4 | C2 domains (2) | Q9HCH3 | CPNE5 | C2 domains (2) |
| O95741 | CPNE6 | C2 domains (2) | Q9UBL6 | CPNE7 | C2 domains (2) | Q86YQ8 | CPNE8 | C2 domains (2) |
| Q8IYJ1 | CPNE9 | C2 domains (2) | Q9Y426 | CU025 | C2 domain (1) | Q5VWQ8 | DAB2P | C2 domain (1) |
| Q14183 | DOC2A | C2 domains (2) | Q14184 | DOC2B | C2 domains (2) | O75923 | DYSF | C2 domains (5) |
| Q9BSJ8 | ESYT1 | C2 domains (5) | A0FGR8 | ESYT2 | C2 domains (3) | A0FGR9 | ESYT3 | C2 domains (3) |
| A9Z1Z3 | FR1L4 | C2 domains (5) | A0AVI2 | FR1L5 | C2 domains (6) | Q2WGJ9 | FR1L6 | C2 domains (6) |
| Q68CZ1 | FTM | C2 domains (2) | Q76N89 | HECW1 | C2 domain (1) | Q9P2P5 | HECW2 | C2 domain (1) |
| Q96PE3 | INP4A | C2 domain (1) | O15327 | INP4B | C2 domain (1) | Q96J02 | ITCH | C2 domain (1) |
| Q15811 | ITSN1 | C2 domain (1) | Q9NZM3 | ITSN2 | C2 domain | Q86YS7 | K0528 | C2 domain (1) |
| | | | | | (1)domains(5) | | | |
| Q8IX03 | KIBRA | C2 domain (1) | P17252 | KPCA | C2 domain (1) | P05771 | KPCB | C2 domain (1) |
| Q05655 | KPCD | C2 domain (1) | Q02156 | KPCE | C2 domain (1) | P05129 | KPCG | C2 domain (1) |
| P24723 | KPCL | C2 domain (1) | Q04759 | KPCT | C2 domain (1) | Q6DN14 | MCTP1 | C2 domains (3) |
| Q6DN12 | MCTP2 | C2 domains (3) | Q9NZM1 | MYOF | C2 domains (5) | P46934 | NEDD4 | C2 domain |
| Q96PU5 | NED4L | C2 domain (1) | Q9UJF2 | NGAP | C2 domain (1) | Q9HC10 | OTOF | C2 domains (4) |
| O00443 | P3C2A | C2 domain (1) | O00750 | P3C2B | C2 domain (1) | O75747 | P3C2G | C2 domain (1) |
| P47712 | PA24A | C2 domain (1) | P0C869 | PA24B | C2 domain (1) | Q86XP0 | PA24D | C2 domain (1) |
| Q3MJ16 | PA24E | C2 domain (1) | Q68DD2 | PA24F | C2 domain (1) | Q9Y6V0 | PCLO | C2 domains (2) |
| P14222 | PERF | C2 domain (1) | Q16512 | PKN1 | C2 domain (1) | Q16513 | PKN2 | C2 domain (1) |
| Q9NQ66 | PLCB1 | C2 domain (1) | Q00722 | PLCB2 | C2 domain (1) | Q01970 | PLCB3 | C2 domain (1) |
| Q15147 | PLCB4 | C2 domain (1) | P51178 | PLCD1 | C2 domain (1) | Q8N3E9 | PLCD3 | C2 domain (1) |
| Q9BRC7 | PLCD4 | C2 domain (1) | Q9P212 | PLCE1 | C2 domain (1) | P19174 | PLCG1 | C2 domain (1) |
| P16885 | PLCG2 | C2 domain (1) | Q4KWH8 | PLCH1 | C2 domain (1) | O75038 | PLCH2 | C2 domain (1) |
| Q15111 | PLCL1 | C2 domain (1) | Q9UPR0 | PLCL2 | C2 domain (1) | Q86YW0 | PLCZ1 | C2 domain (1) |
| C9J798 | RAS4B | C2 domains (2) | P20936 | RASA1 | C2 domain (1) | Q15283 | RASA2 | C2 domains (2) |
| Q14644 | RASA3 | C2 domains (2) | O95294 | RASL1 | C2 domains (2) | O43374 | RASL2 | C2 domains (2) |
| Q86YV0 | RASL3 | C2 domain (1) | Q6WKZ4 | RFIP1 | C2 domain (1) | Q7L804 | RFIP2 | C2 domain (1) |
| Q9BXF6 | RFIP5 | C2 domain (1) | P49796 | RGS3 | C2 domain (1) | Q86UR5 | RIMS1 | C2 domains (2) |
| Q9UQ26 | RIMS2 | C2 domains (2) | Q9UJD0 | RIMS3 | C2 domain (1) | Q9H426 | RIMS4 | C2 domain (1) |
| Q9Y2J0 | RP3A | C2 domains (2) | Q96KN7 | RPGR1 | C2 domain (1) | Q9HCE7 | SMUF1 | C2 domain (1) |
| Q9HAU4 | SMUF2 | C2 domain (1) | Q58G82 | SY14L | C2 domain (1) | Q5VT97 | SYDE2 | C2 domain (1) |
| Q96PV0 | SYGP1 | C2 domain (1) | P21579 | SYT1 | C2 domains (2) | Q6XYQ8 | SYT10 | C2 domains (2) |
| Q9BT88 | SYT11 | C2 domains (2) | Q8IV01 | SYT12 | C2 domains (2) | Q7L8C5 | SYT13 | C2 domains (2) |
| Q8NB59 | SYT14 | C2 domains (2) | Q9BQS2 | SYT15 | C2 domains (2) | Q17RD7 | SYT16 | C2 domains (2) |
| Q9BSW7 | SYT17 | C2 domains (2) | Q8N9I0 | SYT2 | C2 domains (2) | Q9BQG1 | SYT3 | C2 domains (2) |
| Q9H2B2 | SYT4 | C2 domains (2) | O00445 | SYT5 | C2 domains (2) | Q5T7P8 | SYT6 | C2 domains (2) |
| O43581 | SYT7 | C2 domains (2) | Q8NBV8 | SYT8 | C2 domains (2) | Q86SS6 | SYT9 | C2 domains (2) |
| Q8IYJ3 | SYTL1 | C2 domains (2) | Q9HCH5 | SYTL2 | C2 domains (2) | Q4VX76 | SYTL3 | C2 domains (2) |
| Q96C24 | SYTL4 | C2 domains (2) | Q8TDW5 | SYTL5 | C2 domains (2) | Q8N9U0 | TAC2N | C2 domain (1) |
| Q9H0E2 | TOLIP | C2 domain (1) | Q9UPW8 | UN13A | C2 domains (3) | O14795 | UN13B | C2 domains (3) |
| Q8NB66 | UN13C | C2 domains (2) | Q70J99 | UN13D | C2 domains (2) | Q9P2Y5 | UVRAG | C2 domain (1) |
| Q6AWC2 | WWC2 | C2 domain (1) | Q9ULE0 | WWC3 | C2 domain (1) | Q9H0M0 | WWP1 | C2 domain (1) |
| O00308 | WWP2 | C2 domain (1) | Q8NEB9 | PK3C3 | PI3K-type (1) | P42336 | PK3CA | PI3K-type (1) |
| P42338 | PK3CB | PI3K-type (1) | O00329 | PK3CD | PI3K-type (1) | P48736 | PK3CG | PI3K-type (1) |
| O75061 | AUXI | tensin-type (1) | O14976 | GAK | tensin-type (1) | P60484 | PTEN | tensin-type (1) |
| Q63HR2 | TENC1 | tensin-type (1) | Q9HBL0 | TENS1 | tensin-type (1) | Q68CZ2 | TENS3 | tensin-type (1) |
| Q5T6R2 | TPT2L | tensin-type (1) | P56180 | TPTE | tensin-type (1) | Q6XPS3 | TPTE2 | tensin-type (1) |

TABLE 6B
C2 Domain-Containing Human Proteins Listed in UniProtKB/TrEMBL (Unreviewed Proteins)
[Domains: C2 domain(s) = assignment to C2 domain subfamilies not yet done;
number in parentheses = number of C2 domains in protein]

| Entry | Domain(s) | Entry | Domain(s) | Entry | Domain(s) | Entry | Domain(s) |
|---|---|---|---|---|---|---|---|
| A1L3Y1 | C2 domains (2) | A2A284 | C2 domain (1) | A2A2B2 | C2 domains (2) | A2RQD7 | C2 domain (1) |
| A2RUF7 | C2 domain (1) | A4D1A8 | C2 domain (1) | A4D1V5 | C2 domain (1) | A4FU00 | C2 domains (2) |
| A4FU94 | C2 domain (1) | A5PKZ7 | C2 domain (1) | A6H8K5 | C2 domain (1) | A6H8W8 | C2 domain (1) |
| A6NCR4 | C2 domains (2) | A6NGX9 | C2 domain (1) | A6PVH9 | C2 domains (2) | A8CTX8 | C2 domain (1) |
| A8CTY3 | C2 domain (1) | A8K0V7 | C2 domains (2) | A8K112 | C2 domains (2) | A8K2C1 | C2 domains (2) |
| A8K2S1 | C2 domain (1) | A8K539 | C2 domains (2) | A8K7K1 | C2 domains (2) | A8K8A4 | C2 domains (2) |
| A8K8F9 | C2 domain (1) | A8K973 | C2 domains (2) | A8K9T5 | C2 domain (1) | A8KAH3 | C2 domains (2) |
| B0AZL9 | C2 domains (2) | B0QZ18 | C2 domains (2) | B1AM27 | C2 domains (3) | B2R5T1 | C2 domain (1) |
| B2R7R4 | C2 domains (2) | B2R9M7 | C2 domain (1) | B2RCA8 | C2 domains (2) | B2RD40 | C2 domains (2) |
| B3KMV5 | C2 domains (5) | B3KR18 | C2 domain (1) | B3KUZ4 | C2 domain (1) | B3KV15 | C2 domain (1) |
| B3KW89 | C2 domain (1) | B3KWG8 | C2 domain (1) | B3KWK1 | C2 domain (1) | B3KX91 | C2 domains (2) |
| B3KXN8 | C2 domain (1) | B3KXR5 | C2 domain (1) | B4DEH7 | C2 domains (2) | B4DEJ2 | C2 domain (1) |
| B4DEU3 | C2 domains (2) | B4DF98 | C2 domain (1) | B4DG06 | C2 domains (2) | B4DG55 | C2 domains (2) |
| B4DGA2 | C2 domain (1) | B4DGS5 | C2 domain (1) | B4DH42 | C2 domain (1) | B4DIK3 | C2 domains (2) |
| B4DJB2 | C2 domains (2) | B4DK40 | C2 domain (1) | B4DMA3 | C2 domain (1) | B4DMA9 | C2 domain (1) |
| B4DN85 | C2 domain (1) | B4DQA6 | C2 domains (2) | B4DRC6 | C2 domain (1) | B4DRK9 | C2 domains (2) |
| B4DRN7 | C2 domain (1) | B4DRP1 | C2 domains (2) | B4DS63 | C2 domains (2) | B4DTL2 | C2 domain (1) |
| B4DTL8 | C2 domain (1) | B4DTX4 | C2 domain (1) | B4DYZ4 | C2 domain (1) | B4DZI4 | C2 domain (1) |
| B4E065 | C2 domain (1) | B4E2A9 | C2 domains (2) | B4E2P5 | C2 domain (1) | B4E3H3 | C2 domain (1) |
| B4E3M8 | C2 domains (2) | B5BU77 | C2 domain (1) | B7WPN2 | C2 domain (1) | B7Z1M3 | C2 domains (2) |
| B7Z1R9 | C2 domains (2) | B7Z2M0 | C2 domains (2) | B7Z2Q9 | C2 domains (2) | B7Z2R1 | C2 domains (5) |
| B7Z2W4 | C2 domains (2) | B7Z2X0 | C2 domain (1) | B7Z300 | C2 domains (2) | B7Z370 | C2 domains (2) |
| B7Z3G9 | C2 domain (1) | B7Z3S1 | C2 domains (2) | B7Z3V9 | C2 domain (1) | B7Z3W6 | C2 domain (1) |
| B7Z405 | C2 domain (1) | B7Z4G1 | C2 domains (2) | B7Z683 | C2 domain (1) | B7Z6H2 | C2 domain (1) |
| B7Z6K9 | C2 domain (1) | B7Z6S2 | C2 domain (1) | B7Z7W2 | C2 domain (1) | B7Z7Z1 | C2 domain (1) |
| B7Z870 | C2 domain (1) | B7Z8G4 | C2 domains (4) | B7Z8Q0 | C2 domain (1) | B7Z9G3 | C2 domain (1) |
| B7Z9Z3 | C2 domains (2) | B7Z9Z7 | C2 domains (2) | B7ZKJ9 | C2 domains (2) | B7ZKM4 | C2 domains (2) |
| B7ZL55 | C2 domains (2) | B7ZLI3 | C2 domains (3) | B7ZLK1 | C2 domain (1) | B7ZLK2 | C2 domain (1) |
| B7ZLL0 | C2 domain (1) | B7ZM61 | C2 domain (1) | B7ZM86 | C2 domain (1) | B9DI81 | C2 domain (1) |
| B9DI82 | C2 domain (1) | B9DI83 | C2 domain (1) | B9EGH5 | C2 domain (1) | C9IZ68 | C2 domain (1) |
| C9J281 | C2 domain (1) | C9JCN0 | C2 domains (7) | C9JEA7 | C2 domain (1) | C9JK77 | C2 domain (1) |
| C9JR22 | C2 domain (1) | D6R8Z9 | C2 domain (1) | D6RA03 | C2 domains (2) | D6RA42 | C2 domains (3) |
| D6RDY0 | C2 domain (1) | D6RF99 | C2 domain (1) | E1P613 | C2 domain (1) | E2QRH8 | C2 domain (1) |
| E5KVZ4 | C2 domains (2) | E5RG68 | C2 domains (2) | E5RG97 | C2 domain (1) | E5RGM0 | C2 domains (2) |
| E5RHZ0 | C2 domain (1) | E5RI36 | C2 domain (1) | E5RJ85 | C2 domain (1) | E5RJR1 | C2 domains (2) |
| E5RK78 | C2 domains (2) | E6Y8C6 | C2 domain (1) | E7ENC2 | C2 domains (2) | E7ENH5 | C2 domains (2) |
| E7ENV7 | C2 domains (2) | E7ERK1 | C2 domain (1) | E7EU42 | C2 domain (1) | E7EUB9 | C2 domains (2) |
| E9PCB7 | C2 domains (2) | E9PCZ1 | C2 domain (1) | E9PD98 | C2 domain (1) | E9PDN4 | C2 domain (1); |
| | | | | | | | C2 domains (2) |
| E9PF48 | C2 domains (2) | E9PFB6 | C2 domain (1) | E9PGC0 | C2 domain (1) | E9PHF5 | C2 domains (2) |
| E9PJ31 | C2 domain (1) | E9PPL3 | C2 domain (1); | E9PQL8 | C2 domain (1) | E9PS29 | C2 domain (1) |
| | | | C2 domains (2) | | | | |
| F2Z2V0 | C2 domains (2) | F2Z3K9 | C2 domain (1) | F5GWN5 | C2 domain (1) | F5GXN1 | C2 domains (2) |
| F5GXT2 | C2 domains (2) | F5GZC2 | C2 domains (2) | F5GZU9 | C2 domains (2) | F5H090 | C2 domains (2) |
| F5H126 | C2 domains (2) | F5H1N2 | C2 domains (2) | F5H2A1 | C2 domain (1) | F5H2A8 | C2 domains (2) |
| F5H2B0 | C2 domains (2) | F5H2Y6 | C2 domain (1) | F5H3L4 | C2 domain (1) | F5H415 | C2 domain (1) |
| F5H426 | C2 domains (2) | F5H5C4 | C2 domain (1) | F5H5R1 | C2 domain (1) | F5H6C1 | C2 domains (2) |
| F5H7F0 | C2 domains (2) | F5H8B3 | C2 domain (1) | F8VP47 | C2 domains (2) | F8VQX1 | C2 domains (2) |
| F8VRH9 | C2 domains (2) | F8VTU5 | C2 domain (1) | F8VZH8 | C2 domains (3) | F8W059 | C2 domains (3) |
| F8W0P6 | C2 domains (3) | F8W6L0 | C2 domains (3) | F8W6W8 | C2 domains (2) | F8W6X8 | C2 domains (2) |
| F8W7H4 | C2 domains (2) | F8W8J4 | C2 domains (7) | F8W8M9 | C2 domains (3) | F8W9B9 | C2 domains (2) |
| F8WD47 | C2 domains (2) | G3V0F9 | C2 domains (2) | G3V1Y0 | C2 domains (2) | G3V520 | C2 domain (1) |
| G5E960 | C2 domain (1) | H0UI19 | C2 domains (2) | H0Y3G9 | C2 domains (2) | H0Y458 | C2 domain (1) |
| H0Y848 | C2 domain (1); | H0Y8M9 | C2 domain (1) | H0Y9S8 | C2 domains (2) | H0Y9Y6 | C2 domains (2) |
| | C2 domains (2) | | | | | | |
| H0YA70 | C2 domains (2) | H0YBE7 | C2 domain (1) | H0YBU2 | C2 domain (1) | H0YBU6 | C2 domains (2) |
| H0YCJ2 | C2 domain (1) | H0YCQ9 | C2 domain (1); | H0YD14 | C2 domains (3) | H0YE56 | C2 domains (2) |
| | | | C2 domains (2) | | | | |
| H0YGH7 | C2 domain (1) | H0YGY7 | C2 domain (1) | H0YH40 | C2 domain (1) | H0YIH4 | C2 domain (1) |
| H0YJ73 | C2 domains (2) | H0YK35 | C2 domain (1) | H0YK44 | C2 domain (1) | H3BLX3 | C2 domains (2) |
| H3BLZ3 | C2 domain (1) | H3BMD0 | C2 domain (1) | H3BN78 | C2 domains (2) | H3BNF7 | C2 domain (1) |
| H3BQZ6 | C2 domains (2) | H3BRH9 | C2 domains (2) | H3BS47 | C2 domains (2) | H3BSH4 | C2 domain (1) |
| H3BSN9 | C2 domain (1) | H3BSU5 | C2 domains (2) | H3BSX1 | C2 domain (1) | H3BTI1 | C2 domain (1) |
| H3BU41 | C2 domain (1) | H3BUC8 | C2 domain (1) | H3BUD4 | C2 domain (1) | H3BUH8 | C2 domains (2) |
| H3BV03 | C2 domains (2) | H3BVI3 | C2 domains (2) | H7BXE5 | C2 domains (3) | H7BXI1 | C2 domains (3) |
| H7C276 | C2 domain (1) | H7C281 | C2 domain (1) | H7C2Q1 | C2 domains (2) | I3L194 | C2 domain (1) |
| I3L1Z0 | C2 domain (1) | I6L9C3 | C2 domains (2) | I6L9J0 | C2 domains (2) | J3KNP0 | C2 domain (1) |
| J3KP28 | C2 domains (2) | J3KQA0 | C2 domains (2) | J3KQI7 | C2 domains (3) | J3KR03 | C2 domains (2) |
| J3KRN5 | C2 domain (1) | J3QQM4 | C2 domain (1) | J3QRY2 | C2 domain (1) | Q05BS5 | C2 domains (2) |
| Q05DL8 | C2 domains (2) | Q12843 | C2 domain (1) | Q12844 | C2 domain (1) | Q14BD3 | C2 domain (1) |
| Q14BD4 | C2 domain (1) | Q2NKJ5 | C2 domains (3) | Q2NL74 | C2 domains (2) | Q2NNQ7 | C2 domains (7) |
| Q2NNQ8 | C2 domains (5) | Q2TSD3 | C2 domain (1) | Q2Z1P3 | C2 domain (1) | Q307T1 | C2 domain (1) |
| Q32P40 | C2 domain (1) | Q3I768 | C2 domain (1) | Q3MI43 | C2 domain (1) | Q3ZCW0 | C2 domain (1) |
| Q495U1 | C2 domains (2) | Q4FD32 | C2 domains (2) | Q4LE43 | C2 domain (1) | Q4LE65 | C2 domain (1) |
| Q4LE73 | C2 domains (3) | Q53TM3 | C2 domain (1) | Q59EE9 | C2 domain (1) | Q59EZ0 | C2 domain (1) |
| Q59F62 | C2 domain (1) | Q59F77 | C2 domain (1) | Q59FI5 | C2 domain (1) | Q59GK3 | C2 domain (1) |
| Q59H24 | C2 domains (2) | Q5HYD7 | C2 domain (1) | Q5JX44 | C2 domains (2) | Q5JX60 | C2 domains (2) |
| Q5JY20 | C2 domains (2) | Q5JY22 | C2 domains (2) | Q5JY23 | C2 domains (2) | Q5JY24 | C2 domains (2) |
| Q5JYS9 | C2 domain (1) | Q5SSD0 | C2 domain (1) | Q5SSD1 | C2 domains (2) | Q6PJY8 | C2 domains (2) |
| Q6PKD5 | C2 domains (2) | Q6ZNK2 | C2 domain (1) | Q719H8 | C2 domain (1) | Q71SF7 | C2 domains (2) |
| Q71UV9 | C2 domain (1) | Q7Z727 | C2 domain (1) | Q86YU9 | C2 domain (1) | Q8IUP3 | C2 domain (1) |
| Q8IV92 | C2 domain (1) | Q8N1A4 | C2 domain (1) | Q8N7E6 | C2 domains (2) | Q8N7S5 | C2 domain (1) |
| Q8N7X7 | C2 domain (1) | Q8NCP8 | C2 domain (1) | Q8NDM9 | C2 domains (2) | Q9BQS1 | C2 domain (1) |
| Q9UFY1 | C2 domain (1) | | | | | | |

1-15. (canceled)

16. A recombinant clostridial neurotoxin comprising a C2 domain.

17. The recombinant clostridial neurotoxin of claim 16, wherein the C2 domain is (i) a C2 domain present in a protein listed in Table 5, or (ii) a human C2 domain present in a protein listed in Table 6.

18. The recombinant clostridial neurotoxin of claim 1, wherein the C2 domain is a human C2 domain present in a human protein selected from the group consisting of ABR, BAIP3, BCR, C2CD3, C2D1A, C2D1B, CAN5, CAN6, CAPS1, CAPS2, CPNE1, CPNE2, CPNE3, CPNE4, CPNE5, CPNE6, CPNE7, CPNE5, CPNE9, CU025; DAB2P, DOC2A, DOC2B, DYSF, ESYT1, ESYT2, ESYT3; FR1L5, FTM, HECW1, HECW2, ITCH, ITSN1, ITSN2, KPCA, KPCB, KPCE, KPCG, KPCL, MCTP1, MCTP2, MYOF, NEDD4, NED4L, NGAP, OTOF, P3C2A, P3C2B, P3C2G, PA24A, PA24B, PA24D, PA24E, PA24F, PCLO, PERF, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, RASA1, RASA2, RASA3, RAK1, RASL2, RFIP1, RFIP2, RFIP5, RGS3, RIMS1, RIMS2, RIMS3, RIMS4, RP3A, RPGR1, SMUF1, SMUF2, SY14L, SYGP1, SYT1, SYT2, SYT4, SYT5, SYT6, SYT7, SYT8, SYT9, SYT10, SYT11, SYT12, SYT13, SYT14, SYT15, SYT18, SYT17, SYTL1, SYTL2, SYTL3, SYTL4, SYTL5, TAC2N, TOLIP, UN13A, UN13B, UN13C, UN13D, WWC2, WWP1, WWP2, DOC2A, DOC2B, DYSF, ESYT1, ESYT2, ESYT3, KPCA, KPCB, KPCG, MYOF, NED4L, PLCD1, PLCD3, PLCZ1, RFIP1, RFIP2, RFIP5, RP3A, SYT1, SYT2, SYT3, SYT4, SYT5, SYT6, SYT7, SYT9, SYT10, and SYTL1.

19. The recombinant clostridial neurotoxin of claim 16, wherein the clostridial neurotoxin is selected from a Clostridium botulinum neurotoxin serotype A, B, C, D, E, F, and G.

20. The recombinant clostridial neurotoxin of claim 16, wherein the clostridial neurotoxin is selected from Clostridium botulinum neurotoxin serotype A, C and E.

21. The recombinant clostridial neurotoxin of claim 16, wherein the clostridial neurotoxin is a functional variant of a Clostridium botulinum neurotoxin serotype A, B, C, D, E, F, or G.

22. The recombinant clostridial neurotoxin of claim 16, wherein the clostridial neurotoxin is a chimeric Clostridium botulinum neurotoxin, wherein the clostridial neurotoxin light chain and heavy chain are from different clostridial neurotoxin serotypes.

23. The recombinant clostridial neurotoxin of claim 16, wherein the recombinant clostridial neurotoxin comprises an amino acid sequence selected from any one of the sequences set forth in SEQ ID NOs: 33 to 36.

24. The recombinant clostridial neurotoxin of claim 18, wherein the recombinant clostridial neurotoxin shows increased duration of effect relative to an identical clostridial neurotoxin without the C2 domain.

25. A pharmaceutical composition comprising the recombinant clostridial neurotoxin of claim 16.

26. A recombinant single-chain precursor clostridial neurotoxin comprising a C2 domain.

27. The recombinant single-chain precursor clostridial neurotoxin of claim 26, wherein the C2 domain is (i) a C2 domain present in a protein listed in Table 5, or (ii) a human C2 domain present in a human protein listed in Table 6.

28. The recombinant single-chain precursor clostridial neurotoxin of claim 26, wherein the C2 domain is a human C2 domain present in a human protein selected from the group consisting of ABR, BAIP3, BCR, C2CD3, C2D1A, C2D1B, CAN5, CAN6, CAPS1, CAPS2, CPNE1, CPNE2, CPNE3, CPNE4, CPNE5, CPNE6, CPNE7, CPNE8, CPNE9, CU025; DAB2P, DOC2A, DOC2B, DYSF, ESYT1, ESYT2, ESYT3; FR1L5, FTM, HECW1, HECW2, ITCH, ITSN1, ITSN2, KPCA, KPCB, KPCE, KPCG, KPCL, MCTP1, MCTP2, MYOF, NEDD4, NED4L, NGAP, OTOF, P3C2A, P3C2B, P3C2G, PA24A, PA24B, PA24D, PA24E, PA24F, PCLO, PERF, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, RASA1, RASA2, RASA3, RASL1, RASL2, RFIP1, RFIP2, RFIP5, RGS3, RIMS1, RIMS2, RIMS4, RP3A, RPGR1, SMUF1, SMUF2, SY14L, SYGP1, SYT1, SYT2, SYT3, SYT4, SYT5, SYT6, SYT7, SYT8, SYT9, SYT10, SYT11, SYT12, SYT13, SYT14, SYT15, SYT16, SYT17, SYTL1, SYTL2, SYTL3, SYTL4, SYTL5, TAC2N, TOLIP, UN13A, UN13B, UN13C, UN13D, WWC2, WWP1, WWP2, DOC2A, DOC2B, DYSF, ESYT1, ESYT2, ESYT3, FR1L5, KPCA, KPCB, KPCG, MYOF, NED4L, PLCD1, PLCD3, PLCZ1, RFIP1, RFIP2, RFIP5, RP3A, SYT1, SYT2, SYT3, SYT4, SYT5, SYT6, SYT7, SYT9, SYT10, and SYTL1.

29. The recombinant single-chain precursor clostridial neurotoxin of claim 26, wherein the clostridial neurotoxin is selected from a Clostridium botulinum neurotoxin serotype A, B, C, D, E, F, and G.

30. The recombinant single-chain precursor clostridial neurotoxin of claim 26, wherein the clostridial neurotoxin is selected from Clostridium botulinum neurotoxin serotype A, C and E.

31. The recombinant single-chain precursor clostridial neurotoxin of claim 26, wherein the clostridial neurotoxin is a functional variant of a Clostridium botulinum neurotoxin serotype A, B, C, D, E, F, or G.

32. The recombinant single-chain precursor clostridial neurotoxin of claim 26, wherein the clostridial neurotoxin is a chimeric Clostridium botulinum neurotoxin, wherein the clostridial neurotoxin light chain and heavy chain are from different clostridial neurotoxin serotypes.

33. The recombinant single-chain precursor clostridial neurotoxin of claim 26, wherein the recombinant clostridial neurotoxin comprises an amino acid sequence selected from any one of the sequences set forth in SEQ ID NOs: 33 to 36.

34. A nucleic acid encoding the recombinant single-chain precursor clostridial neurotoxin of claim 26.

35. The nucleic acid of claim 34, comprising a nucleic acid sequence selected from any one of the sequences set forth in SEQ ID NOs: 37 to 68.

36. The nucleic acid of claim 34, comprising a nucleic acid sequence selected from any one of the sequences set forth in SEQ ID NOs: 69 to 72.

37. A method for obtaining the nucleic acid of claim 26, comprising the step of inserting a nucleic acid encoding a C2 domain into a nucleic acid encoding a parental clostridial neurotoxin.

38. A vector comprising the nucleic acid of claim 26.

39. A recombinant host cell comprising the nucleic acid of claim 26.

40. A method for producing the recombinant single-chain precursor clostridial neurotoxin of claim 26, comprising the step of expressing a nucleic acid encoding the recombinant single-chain precursor clostridial neurotoxin comprising a C2 domain in a host cell, cultivating the host cell under conditions which result in the expression of the nucleic acid and recombinant single-chain precursor clostridial neurotoxin, and recovering the recombinant single-chain precursor clostridial neurotoxin.

41. A method of treating a disease requiring improved chemodenervation, comprising administering the recombinant clostridial neurotoxin of claim 16 to a subject in need thereof, wherein the recombinant clostridial neurotoxin exhibits longer lasting denervation relative to an identical clostridial neurotoxin without a C2 domain.

42. A method of cosmetic treatment comprising administering the recombinant clostridial neurotoxin of claim 16 to a subject, wherein the recombinant clostridial neurotoxin exhibits longer lasting denervation relative to an identical clostridial neurotoxin without a C2 domain.


SEQUENCE LISTING (72 sequences)

SEQ ID NO: 1 - protein, 106 aa, Homo sapiens
SLQCTIIKAKGLKPMDSNGLADPYVKLHLLPGASKSNKLRTKTLRNTRNPIWNETLVYHGITDEDMQRKTLRISVCDEDKFGHNEFIGETRFSLKKLKPNQRKNFN

SEQ ID NO: 2 - protein, 115 aa, Homo sapiens
GLIVGIIRCVHLAAMDANGYSDPFVKLWLKPDMGKKAKHKTQIKKKTLNPEFNEEFFYDIKHSDLAKKSLDISVWDYDIGKSNDYIGGCQLGISAKGERLKHWYECLKNKDKKIE

SEQ ID NO: 3 - protein, 106 aa, Homo sapiens
TLHCSILRAKGLKPMDFNGLADPYVKLHLLPGACKANKLKTKTQRNTLNPVWNEDLTYSGITDDDITHKVLRIAVCDEDKLSHNEFIGEIRVPLRRLKPSQKKHFN

SEQ ID NO: 4 - protein, 106 aa, Homo sapiens
ALHCTITKAKGLKPMDHNGLADPYVKLHLLPGASKANKLRTKTLRNTLNPTWNETLTYYGITDEDMIRKTLRISVCDEDKFRHNEFIGETRVPLKKLKPNHTKTFS

SEQ ID NO: 5 - protein, 102 aa, Homo sapiens
ELRVHVIQCQGLAAARRRRSDPYVKSYLLPDKQSKRKTAVKKRNLNPVFNETLRYSVPQAELQGRVLSLSVWHRESLGRNIFLGEVEVPLDTWDWGSEPTWL

SEQ ID NO: 6 - protein, 104 aa, Homo sapiens
ILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMER

SEQ ID NO: 7 - protein, 104 aa, Homo sapiens
KLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWY

SEQ ID NO: 8 - protein, 104 aa, Homo sapiens
VLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWF

SEQ ID NO: 9 - protein, 104 aa, Homo sapiens
EIHVTVGEARNLIPMDPNGLSDPYVKLKLIPDPRNLTKQKTRTVKATLNPVWNETFVFNLKPGDVERRLSVEVWDWDRTSRNDFMGAMSFGVSELLKAPVDGWY

SEQ ID NO: 10 - protein, 106 aa, Homo sapiens
RLNIRVISGQQLPKVNKNKNSIVDPKVTVEIHGVSRDVASRQTAVITNNGFNPWWDTEFAFEVVVPDLALIRFLVEDYDASSKNDFIGQSTIPLNSLKQGYRHVHL

SEQ ID NO: 11 - protein, 107 aa, Homo sapiens
TLSIQVLTAQQLPKLNAEKPHSIVDPLVRIEIHGVPADCARQETDYVLNNGFNPRWGQTLQFQLRAPELALVRFVVEDYDATSPNDFVGQFTLPLSSLKQGYRHIHL

SEQ ID NO: 12 - protein, 105 aa, Homo sapiens
TLTIRLISGIQLPLTHSSSNKGDSLVIIEVFGVPNDQMKQQTRVIKKNAFSPRWNETFTFIIHVPELALIRFVVEGQGLIAGNEFLGQYTLPLLCMNKGYRRIPL

SEQ ID NO: 13 - protein, 162 aa, Homo sapiens
YRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTK

SEQ ID NO: 14 - protein, 105 aa, Homo sapiens
HVQVTVLQARGLRAKGPGGTSDAYAVIQVGKEKYATSVSERSLGAPVWREEATFELPSLLSSGPAAAATLQLTVLHRALLGLDKFLGRAEVDLRDLHRDQGRRKT

SEQ ID NO: 15 - protein, 124 aa, Homo sapiens
HVQVTVLRARGLRGKSSGAGSTSDAYTVIQVGREKYSTSVVEKTHGCPEWREECSFELPPGALDGLLRAQEADAGPAPWAASSAAACELVLTTMHRSLIGVDKFLGQATVALDEVFGAGRAQHT

SEQ ID NO: 16 - protein, 104 aa, Homo sapiens
HVQVTVLQAKDLKPKGKSGTNDTYTIIQLGKEKYSTSVAEKTLEPVWKEEASFELPGLLIQGSPEKYILFLIVMHRSLVGLDKFLGQVAINLNDIFEDKQRRKT

SEQ ID NO: 17 - protein, 103 aa, Homo sapiens
QLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEW

SEQ ID NO: 18 - protein, 115 aa, Homo sapiens
KLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIA

SEQ ID NO: 19 - protein, 103 aa, Homo sapiens
QLTVGVLQAAELPALDMGGTSDPYVKVFLLPDKKKKYETKVHRKTLNPAFNETFTFKVPYQELGGKTLVMAIYDFDRFSKHDIIGEVKVPMNTVDLGQPIEEW

SEQ ID NO: 20 - protein, 104 aa, Homo sapiens
QLVVRILQALDLPAKDSNGFSDPYVKIYLLPDRKKKFQTKVHRKTLNPVFNETFQFSVPLAELAQRKLHFSVYDFDRFSRHDLIGQVVLDNLLELAEQPPDRPL

SEQ ID NO: 21 - protein, 105 aa, Homo sapiens
AFVVNIKEARGLPAMDEQSMTSDPYIKMTILPEKKHKVKTRVLRKTLDPAFDETFTFYGIPYTQIQELALHFTILSFDRFSRDDIIGEVLIPLSGIELSEGKMLM

SEQ ID NO: 22 - protein, 103 aa, Homo sapiens
QLLVGILQAMGLAALDLGGSSDPYVRVYLLPDKRRRYETKVHRQTLNPHFGETFAFKVPYVELGGRVLVMAVYDFDRFSRNDAIGEVRVPMSSVDLGRPVQAW

SEQ ID NO: 23 - protein, 105 aa, Homo sapiens
TLIVRILKAFDLPAKDFCGSSDPYVKIYLLPDRKCKLQTRVHRKTLNPTFDENFHFPVPYEELADRKLHLSVFDFDRFSRHDMIGEVILDNLFEASDLSRETSIW

SEQ ID NO: 24 - protein, 104 aa, Homo sapiens
TLTVKIMKAQELPAKDFSGTSDPFVKIYLLPDKKHKLETKVKRKNLNPHWNETFLFEGFPYEKVVQRILYLQVLDYDRFSRNDPIGEVSIPLNKVDLTQMQTFW

SEQ ID NO: 25 - protein, 104 aa, Homo sapiens
QLIVKIHKAVNLPAKDFSGTSDPYVKIYLLPDRKTKHQTKVHRKTLNPVFDEVFLFPVPYNDLEARKLHFSVYDFDRFSRHDLIGQVVVDHFLDLADFPRECIL

SEQ ID NO: 26 - protein, 104 aa, Homo sapiens
LLVVKIIKALDLPAKDFTGTSDPYVKMYLLPDRKKKFQTRVHRKTLNPLFDETFQFPVAYDQLSNRKLHFSVYDFDRFSRHDMIGEVILDNLFEVSDLSREATV

SEQ ID NO: 27 - protein, 106 aa, Homo sapiens
KLVSIVHGCRSLRQNGRDPPDPYVSLLLLPDKNRGTKRRTSQKKRTLSPEFNERFEWELPLDEAQRRKLDVSVKSNSSFMSRERELLGKVQLDLAETDLSQGVARW

SEQ ID NO: 28 - protein, 129 aa, Homo sapiens
LGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGT

SEQ ID NO: 29 - protein, 108 aa, Homo sapiens
CLSVLINGCRNLTPCTSSGADPYVRVYLLPERKWACRKKTSVKRKTLEPLFDETFEFFVPMEEVKKRSLDVAVKNSRPLGSHRRKELGKVLIDLSKEDLIKGFSQWYE

SEQ ID NO: 30 - protein, 101 aa, Homo sapiens
MLCCLLVRASNLPSAKKDRRSDPVASLTFRGVKKRTKVIKNSVNPVWNEGFEWDLKGIPLDQGSELHVVVKDHETMGRNRFLGEAKVPLREVLATPSLSAS

SEQ ID NO: 31 - protein, 100 aa, Homo sapiens
MLRVIVESASNIPKTKFGKPDPIVSVIFKDEKKKTKKVDNELNPVWNEILEFDLRGIPLDFSSSLGIIVKDFETIGQNKLIGTATVALKDLTGDQSRSLP

SEQ ID NO: 32 - protein, 95 aa, Homo sapiens
QVRVKVFEARQLMGNNIKPVVKVSIAGQQHQTRIKMGNNPFFNEIFFQNFHEVPAKFFDETILIQTDIGFIYHSPGHTLLRKWLGLCQPNNPGSG

SEQ ID NO: 33 - protein, 1408 aa, Artificial Sequence
  single-chain precursor protein with C2 domain
MSLQCTIIKAKGLKPMDSNGLADPYVKLHLLPGASKSNKLRTKTLRNTRNPIWNETLVYHGITDEDMQRKTLRISVCDEDKFGHNEFIGETRFSLKKLKPNQRKNFNPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 34 - protein, 1431 aa, Artificial Sequence
  single-chain precursor protein with C2 domain
MLGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGTPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 35 - protein, 1408 aa, Artificial Sequence
  single-chain precursor protein with C2 domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSLQCTIIKAKGLKPMDSNGLADPYVKLHLLPGASKSNKLRTKTLRNTRNPIWNETLVYHGITDEDMQRKTLRISVCDEDKFGHNEFIGETRFSLKKLKPNQRKNFNSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 36 - protein, 1431 aa, Artificial Sequence
  single-chain precursor protein with C2 domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITLGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGTSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 37 - DNA, 318 bp, Homo sapiens
tccctgcagtgcaccatcattaaggccaagggcctgaagcccatggattcaaacggcttggctgatccctacgttaagctgcacctcctgccgggagccagcaagtccaacaagcttcgtacaaaaactctgcggaatacccggaaccccatctggaatgagaccctcgtgtatcacggcatcaccgatgaggacatgcaaaggaagaccctcaggatctccgtctgtgatgaggacaaatttggccacaatgaatttattggtgagaccagattctccctcaagaaactgaagcccaaccagaggaagaatttcaac

SEQ ID NO: 38 - DNA, 345 bp, Homo sapiens
ggcctcattgtgggcatcatacgctgcgtgcacctggctgccatggacgctaatggctactcagacccattcgtcaagctctggctgaaaccggacatgggaaagaaggccaaacacaagactcaaattaaaaagaaaaccttgaatcccgaattcaatgaggagtttttctatgacatcaaacacagtgacctggcaaagaagtcactggacatttcagtctgggactatgacatcggcaagtccaatgattacatcggaggctgccagctggggatctctgccaagggagagcgcttaaaacactggtacgagtgtctgaaaaataaagacaagaagatagag

SEQ ID NO: 39 - DNA, 318 bp, Homo sapiens
actctgcactgtagcatcctcagggccaagggcctcaagcccatggatttcaatggcctcgccgacccctacgtcaagctgcacttgctgcctggagcctgtaaggccaataagctaaaaacgaagactcagaggaacacactgaatcccgtgtggaatgaggacctgacttacagcgggatcacagatgacgacatcacgcacaaggtgctcaggatcgccgtctgtgatgaggacaagctgagtcacaatgagtttattggggagatccgcgtgcccctccgccgcctcaagccttcgcagaagaagcattttaac

SEQ ID NO: 40 - DNA, 318 bp, Homo sapiens
gccctccactgcaccatcaccaaggccaagggcctgaagccaatggaccacaatgggctggcagacccctacgtcaagctgcacctgctgccaggagccagtaaggcaaataagctcagaacaaaaactctccgtaacactctgaaccccacatggaacgagaccctcacttactacgggatcacagatgaagacatgatccgcaagaccctgcggatctctgtgtgtgacgaggacaaattccggcacaatgagttcatcggggagacacgtgtgcccctgaagaagctgaaacccaaccacaccaagaccttcagc

SEQ ID NO: 41 - DNA, 306 bp, Homo sapiens
gagctgcgcgtgcacgtgatccagtgccagggcctggccgccgcccggcgccgccgctcggacccctacgtcaaaagctacctcctcccggataagcagagcaagcgcaagacggcggtgaagaaacggaatctgaatccggttttcaacgagactctccggtactccgtcccgcaggccgagcttcagggccgcgtgctgagcctgtctgtgtggcaccgcgaaagcctgggtcgcaacatctttctgggcgaagttgaagtgcccctggacacgtgggactggggctctgagcccacctggctc

SEQ ID NO: 42 - DNA, 312 bp, Homo sapiens
attctcagagtaaaagttgtttctggaattgatctcgccaaaaaggacatctttggagccagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcga

SEQ ID NO: 43 - DNA, 312 bp, Homo sapiens
aagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtac

SEQ ID NO: 44 - DNA, 312 bp, Homo sapiens
gtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggttt

SEQ ID NO: 45 - DNA, 312 bp, Homo sapiens
gagatccacgtaactgttggcgaggcccgtaacctaattcctatggaccccaatggtctctctgatccctatgtgaaactgaagctcatcccagaccctcggaacctgacgaaacagaagacccgaacggtgaaagccacgctaaaccctgtgtggaatgagacctttgtgttcaacctgaagccaggggatgtggagcgccggctcagcgtggaggtgtgggactgggaccggacctcccgcaacgacttcatgggggccatgtcctttggcgtctcggagctgctcaaggcgcccgtggatggctggtac

SEQ ID NO: 46 - DNA, 318 bp, Homo sapiens
cggctcaacatcagggtcatttcggggcagcagctgccaaaagtcaacaagaataagaattcaattgtggaccccaaagtgacagtggagatccatggcgtgagccgggacgtggccagccgccagactgctgtcatcaccaacaatggtttcaacccatggtgggacacggagtttgcgtttgaggtagttgtgcctgaccttgccctcatccgcttcttggtggaagattatgatgcctcctccaagaatgacttcattggccagagtaccatccccttgaacagcctcaagcaaggataccgccatgtccacctc

SEQ ID NO: 47 - DNA, 321 bp, Homo sapiens
actctcagcatccaggtgctgactgcacagcagctgcccaagctgaatgccgagaagccacactccattgtggaccccctggtgcgcattgagatccatggggtgcccgcagactgtgcccggcaggagactgactacgtgctcaacaatggcttcaacccccgctgggggcagaccctgcagttccagctgcgggctccggagctggcactggtccggtttgtggtggaagattatgacgccacctcccccaatgactttgtgggccagtttacactgcctcttagcagcctaaagcaagggtaccgccacatacacctg

SEQ ID NO: 48 - DNA, 315 bp, Homo sapiens
acacttacaataaggctcatcagtggtatccagttgcctcttactcattcatcatctaacaaaggtgattcattagtaattatagaagtttttggtgttccaaatgatcaaatgaagcagcagactcgtgtaattaaaaaaaatgcttttagtccaagatggaatgaaacattcacatttattattcatgtcccagaattggcattgatacgttttgttgttgaaggtcaaggtttaatagcaggaaatgaatttcttgggcaatatactttgccacttctatgcatgaacaaaggttatcgtcgtattcctctg

SEQ ID NO: 49 - DNA, 486 bp, Homo sapiens
tatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaa

SEQ ID NO: 50 - DNA, 315 bp, Homo sapiens
cacgtgcaggtgacggtgctgcaggcgcggggcctgcgggccaagggccccgggggcacgagcgacgcgtacgcggtgatccaggtgggcaaggagaagtacgccacctccgtgtcggagcgcagcctgggcgcgcccgtgtggcgcgaggaggccaccttcgagctgccatcgctgctgtcctccggacccgcggccgccgccaccctgcagctcaccgtgctgcaccgcgcgctgctcggcctcgacaagttcctgggccgcgccgaggtggacctgcgggatctgcaccgcgaccagggccgcaggaagacg

SEQ ID NO: 51 - DNA, 372 bp, Homo sapiens
cacgtccaggtgacggtgctgcgggcccgcgggctgcggggcaagagctcgggagcgggcagcaccagcgacgcgtacacggtgatccaggtgggccgcgagaagtacagtacgtcggtggtggagaagacgcacggctgccccgagtggcgtgaggagtgctccttcgagctgccgccgggggccctggatggcctgctgcgggcgcaggaggccgacgcgggcccggcgccctgggccgcgagctccgccgccgcctgcgagctggtgctcaccaccatgcaccgctcgctcatcggcgtcgacaagttcctgggccaggccacggtggcgctggacgaggtcttcggcgcaggccgcgcccagcacacg

SEQ ID NO: 52 - DNA, 312 bp, Homo sapiens
cacgtgcaggtcacagtgctccaagccaaagatctgaagccaaaaggcaaaagtggtaccaatgacacatacactataattcagctgggcaaggaaaagtactccacctctgtagctgagaaaacccttgagccagtttggaaggaggaggcctctttcgagctacctggattgctaattcagggaagtccagagaaatacattcttttccttatagttatgcacaggtccctggtgggtctggataaatttttagggcaggtggcaatcaatctcaatgacatctttgaggacaaacaaagaaggaaaaca

SEQ ID NO: 53 - DNA, 309 bp, Homo sapiens
cagctgctggtagggatcattcaggctgccgaactgcccgccttggacatggggggcacatctgatccttacgtgaaagtgtttctgctacctgataagaagaagaaatttgagacaaaagtccaccgaaaaacccttaatcctgtcttcaatgagcaatttactttcaaggtaccatactcggaattgggtggcaaaaccctagtgatggctgtatatgattttgatcgtttctctaagcatgacatcattggagaatttaaagtccctatgaacacagtggattttggccatgtaactgaggaatgg

SEQ ID NO: 54 - DNA, 345 bp, Homo sapiens
aagctgactgttgtcattctggaggcaaagaacctgaagaagatggatgtgggtggcttatccgatccttatgtgaagattcatctgatgcagaatggtaagaggctgaagaagaaaaagacaacaattaaaaagaacacacttaacccctactacaatgagtcattcagctttgaagtaccttttgaacaaatccagaaagtgcaggtggtggtaactgttttggactatgacaagattggcaagaacgatgccatcggcaaagtctttgtgggctacaacagcaccggcgcggagctgcgacactggtcagacatgctggccaaccccaggcgacctattgcc

SEQ ID NO: 55 - DNA, 309 bp, Homo sapiens
cagcttactgtgggcgttctgcaggctgctgaactgcctgccctggacatgggaggcacctcagacccttatgtcaaggtcttcctccttcctgacaagaagaagaaatatgagaccaaagtccatcggaagacactgaaccctgccttcaatgaaaccttcaccttcaaggtgccataccaggagcttgggggcaaaactctggtgatggccatctatgactttgaccgcttctccaaacatgacatcattggagaggtaaaggtgcctatgaacacagtggacctcggccagcccattgaggagtgg

SEQ ID NO: 56 - DNA, 312 bp, Homo sapiens
cagctggtggtgaggatcctgcaggccctggacctccctgccaaggactccaacggcttctcagacccctacgtcaagatctacctgctgcctgaccgcaagaaaaagtttcagaccaaggtgcacaggaagaccctgaaccccgtcttcaatgagacgtttcaattctcggtgcccctggccgagctggcccaacgcaaactgcacttcagcgtctatgactttgaccgcttctcgcggcacgacctcatcggccaggtggtgctggacaacctcctggagctggccgagcagccccctgaccgcccgctc

SEQ ID NO: 57 - DNA, 315 bp, Homo sapiens
gcatttgtggtcaatatcaaggaagcccgtggcttgccagccatggatgagcagtcgatgacctctgacccatatatcaaaatgacgatcctcccagagaagaagcataaagtgaaaactagagtgctgagaaaaaccttggatccagcttttgatgagacctttacattctatgggataccctacacccaaatccaagaattggccttgcacttcacaattttgagttttgacaggttttcaagagatgatatcattggggaagttctaattcctctctcgggaattgaattatctgaaggaaaaatgttaatg

SEQ ID NO: 58 - DNA, 309 bp, Homo sapiens
cagctgctggtgggcattctgcaagcaatgggattggcagccttggatcttggtggctcctcggacccctatgtgcgggtctacctgctgccggacaaacggaggcggtacgagaccaaggtgcatcggcagacgctgaaccctcactttggggagaccttcgccttcaaggtcccctacgtggagctggggggcagggtgctggtcatggcggtgtacgacttcgaccgcttctctcgcaatgacgccatcggggaggtgcgggtccctatgagctccgtggacctggggcggccagtgcaggcctgg

SEQ ID NO: 59 - DNA, 315 bp, Homo sapiens
accctgattgtgcgtatcctgaaggcttttgacctccctgccaaggacttttgtggaagctctgacccttatgtcaagatctacctcctgcctgaccgcaaatgcaagctgcagacccgggtgcaccgcaagaccctgaaccccacctttgatgagaacttccacttccctgtgccctatgaggagctggctgaccgcaagctgcatctcagtgtcttcgactttgaccgcttctcccgccatgacatgattggcgaggtcatcctggacaacctctttgaggcctctgacctgtctcgggaaacctccatctgg

SEQ ID NO: 60 - DNA, 312 bp, Homo sapiens
acgctcaccgtgaagatcatgaaggcccaggagctgccggccaaggacttcagcggcaccagcgaccccttcgtcaagatctacctgctgcccgacaagaagcacaagctggagaccaaggtgaagcggaagaacctgaacccccactggaacgagaccttcctctttgaaggttttccctatgagaaggtggtgcagaggatcctctacctccaagtcctggactatgaccgcttcagccgcaacgaccccattggggaggtgtccatcccccttaacaaggtggacctgacccagatgcagaccttctgg

SEQ ID NO: 61 - DNA, 312 bp, Homo sapiens
cagctcatagtgaagattcacaaagctgtcaatttgcccgccaaggacttttctgggacttcagatccttatgtcaagatctatttgcttcctgatcggaaaacaaaacaccagactaaagttcacagaaagaccctgaaccctgtgtttgatgaagtgtttttatttccggttccctacaatgaccttgaagcacggaagcttcacttctctgtgtacgactttgacaggttctctcgtcatgacttaatcggccaagtggtggtggatcacttcctagacttggctgatttccccagggagtgcatcctt

SEQ ID NO: 62 - DNA, 312 bp, Homo sapiens
cttctagttgttaaaattatcaaagctttagatctccctgctaaagacttcacaggaacttctgacccttatgtgaagatgtatcttcttccagataggaaaaagaaatttcagacccgcgtgcacagaaagactttaaatcctctatttgatgaaacttttcaatttcctgtagcatatgatcaactaagcaaccgaaaactacatttcagtgtgtatgattttgacagattttctagacatgacatgattggggaagtgattcttgataatttgtttgaagtctctgatctctccagggaagccacagta

SEQ ID NO: 63 - DNA, 318 bp, Homo sapiens
aagctggtcagcattgttcatggttgccggtcccttcgacagaatggacgtgatcctcctgatccctatgtgtcactgttgctactgccagacaagaaccgaggcaccaagaggaggacctcacagaagaagaggaccctgagtcctgaatttaatgaacggtttgagtgggaactccccctggatgaggcccagagacgaaagctggatgtctctgtcaagtctaattcctccttcatgtcaagagagcgtgagctgctggggaaggtgcagctggacctagctgagacagacctttcccagggtgtagcccggtgg

SEQ ID NO: 64 - DNA, 387 bp, Homo sapiens
ctggggcagatccagctgaccatccggcacagctcgcagagaaacaagcttatcgtggtcgtgcatgcctgcagaaacctcattgccttctctgaagacggctctgacccctatgtccgcatgtatttattaccagacaagaggcggtcaggaaggaggaaaacacacgtgtcaaagaaaacattaaatccagtgtttgatcaaagctttgatttcagtgtttcgttaccagaagtgcagaggagaacgctcgacgttgccgtgaagaacagtggcggcttcctgtccaaagacaaagggctccttggcaaagtattggttgctctggcatctgaagaacttgccaaaggctggacccagtggtatgacctcacggaagatgggacg

SEQ ID NO: 65 - DNA, 324 bp, Homo sapiens
tgcctcagcgtgctaatcaatggctgcagaaacctaacaccatgtaccagcagtggagctgatccctacgtccgtgtctacttgttgccagaaaggaagtgggcatgtcgtaagaagacttcagtgaagcggaagaccttggaacccctgtttgatgagacatttgaattttttgttcccatggaagaagtaaagaagaggtcactagatgttgcagtgaaaaatagtaggccacttggctcacacagaagaaaggagttaggaaaagtactgattgacttatcaaaagaagatctgattaagggcttttcacaatggtatgag

SEQ ID NO: 66 - DNA, 303 bp, Homo sapiens
atgctgtgctgcctgctggtgagggccagcaacctccccagtgcgaagaaggaccggcgcagcgaccctgtcgcaagcctgactttccgaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagc

SEQ ID NO: 67 - DNA, 300 bp, Homo sapiens
atgctgcgagtgattgtggaatctgccagcaatatccctaaaacgaaatttggcaagccggatcctattgtttctgtcatttttaaggatgagaaaaagaaaacaaagaaagttgataatgaattgaaccctgtctggaatgagattttggagtttgacttgaggggtataccactggacttttcatcttcccttgggattattgtgaaagattttgagacaattggacaaaataaattaattggcacggcgactgtagccctgaaggacctgactggtgaccagagcagatccctgccg

SEQ ID NO: 68 - DNA, 285 bp, Homo sapiens
caggttcgagtgaaggtgtttgaagcccgacagctcatgggcaacaacatcaaaccagtggtgaaggtgtccatcgcaggccagcagcaccagacacgcatcaagatgggaaacaaccctttctttaatgagatcttcttccagaattttcatgaggttcctgcaaagttctttgatgagaccatcttaatccagacagatattgggtttatctaccattctccaggtcacacactcctaaggaaatggctaggcctctgccagccaaataaccctggcagtggt

SEQ ID NO: 69 - DNA, 4227 bp, Artificial Sequence
  DNA encoding single-chain precursor protein with C2 domain
atgtccctgcagtgcaccatcattaaggccaagggcctgaagcccatggattcaaacggcttggctgatccctacgttaagctgcacctcctgccgggagccagcaagtccaacaagcttcgtacaaaaactctgcggaatacccggaaccccatctggaatgagaccctcgtgtatcacggcatcaccgatgaggacatgcaaaggaagaccctcaggatctccgtctgtgatgaggacaaatttggccacaatgaatttattggtgagaccagattctccctcaagaaactgaagcccaaccagaggaagaatttcaacccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtgcgcggcatcattaccagcaaggcaggtgcgggcaagtccttggttccgcgtggcagcgccggcgccggcgcgctcaatgatctgtgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 70 - DNA, 4296 bp, Artificial Sequence
  DNA encoding single-chain precursor protein with C2 domain
atgctggggcagatccagctgaccatccggcacagctcgcagagaaacaagcttatcgtggtcgtgcatgcctgcagaaacctcattgccttctctgaagacggctctgacccctatgtccgcatgtatttattaccagacaagaggcggtcaggaaggaggaaaacacacgtgtcaaagaaaacattaaatccagtgtttgatcaaagctttgatttcagtgtttcgttaccagaagtgcagaggagaacgctcgacgttgccgtgaagaacagtggcggcttcctgtccaaagacaaagggctccttggcaaagtattggttgctctggcatctgaagaacttgccaaaggctggacccagtggtatgacctcacggaagatgggacgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtgcgcggcatcattaccagcaaggcaggtgcgggcaagtccttggttccgcgtggcagcgccggcgccggcgcgctcaatgatctgtgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 71 - DNA, 4227 bp, Artificial Sequence
  DNA encoding single-chain precursor protein with C2 domain
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtgcgcggcatcattacctccctgcagtgcaccatcattaaggccaagggcctgaagcccatggattcaaacggcttggctgatccctacgttaagctgcacctcctgccgggagccagcaagtccaacaagcttcgtacaaaaactctgcggaatacccggaaccccatctggaatgagaccctcgtgtatcacggcatcaccgatgaggacatgcaaaggaagaccctcaggatctccgtctgtgatgaggacaaatttggccacaatgaatttattggtgagaccagattctccctcaagaaactgaagcccaaccagaggaagaatttcaacagcaaggcaggtgcgggcaagtccttggttccgcgtggcagcgccggcgccggcgcgctcaatgatctgtgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 72 - DNA, 4296 bp, Artificial Sequence
  DNA encoding single-chain precursor protein with C2 domain
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtgcgcggcatcattaccctggggcagatccagctgaccatccggcacagctcgcagagaaacaagcttatcgtggtcgtgcatgcctgcagaaacctcattgccttctctgaagacggctctgacccctatgtccgcatgtatttattaccagacaagaggcggtcaggaaggaggaaaacacacgtgtcaaagaaaacattaaatccagtgtttgatcaaagctttgatttcagtgtttcgttaccagaagtgcagaggagaacgctcgacgttgccgtgaagaacagtggcggcttcctgtccaaagacaaagggctccttggcaaagtattggttgctctggcatctgaagaacttgccaaaggctggacccagtggtatgacctcacggaagatgggacgagcaaggcaggtgcgggcaagtccttggttccgcgtggcagcgccggcgccggcgcgctcaatgatctgtgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa